(19) United States
(12) Patent Application Publication
(10) Pub. No.: US 2024/0287614 A1
(43) Pub. Date: Aug. 29, 2024

(54) RECIPROCAL MICRORNA-MRNA PAIRINGS AS BIOMARKERS AND THERAPEUTIC TARGETS IN CANCER

(71) Applicant: UNIVERSITY OF MARYLAND, EASTERN SHORE, Princess Anne, MD (US)

(72) Inventors: Bi-Dar Wang, Salisbury, MD (US); Himali Gujrati, Salisbury, MD (US); Siyoung Ha, Salisbury, MD (US)

(21) Appl. No.: 18/441,787

(22) Filed: Feb. 14, 2024

Related U.S. Application Data
(60) Provisional application No. 63/520,083, filed on Aug. 16, 2023.
(60) Provisional application No. 63/484,859, filed on Feb. 14, 2023.

(51) Int. Cl. C12Q 1/6886; C12Q 1/6851; G01N 33/574

(52) U.S. Cl. CPC C12Q 1/6886; C12Q 1/6851; G01N 33/57434; C12Q 2600/106; C12Q 2600/178

(57) ABSTRACT

The present disclosure relates to the involvement of miR-34a-5p, miR-99b-5p, and miR-96-5p in certain cancers, as well as the use of agonists or antagonists thereof to treat the same.

### CROSS REFERENCE TO RELATED APPLICATIONS

This application claims priority to provisional applications U.S. Ser. No. 63/484,859 filed Feb. 14, 2023, and U.S. Ser. No. 63/520,083 filed Aug. 16, 2023, which are incorporated herein by reference in their entireties.

### GOVERNMENT SUPPORT

This invention was made with government support under SC1GM127256 awarded by the National Institutes of Health (NIH). The government has certain rights in the invention.

### SEQUENCE LISTING XML

The instant application contains a sequence listing, which has been submitted in XML file format by electronic submission and is hereby incorporated by reference in its entirety. The XML file, created on Feb. 14, 2024, is named P14693US02.xml and is 17,575 bytes in size.

### TECHNICAL FIELD

The present disclosure relates generally to the fields of oncology, molecular biology, and medicine. More particularly, the disclosure relates to use of certain miRNAs that are dysregulated in prostate cancer and in aggressive cancers in general as both diagnostic and therapeutic targets.

### BACKGROUND

Cancer has progressively been a major global health concern. Prostate cancer is now the most frequently diagnosed cancer and the second leading cause of cancer deaths among American men. Notably, African Americans are 1.6 times more likely to develop prostate cancer, and 2.4 times more likely to die from this disease compared to their European American counterparts. Multiple socioeconomic factors have been postulated to explain the observed prostate cancer disparities. However, higher mortality and recurrence is still observed in African Americans after adjustment for socioeconomic status, indicating that intrinsic biological differences account for at least part of the prostate cancer disparities.

### SUMMARY

Methods of treating cancer in a subject in need thereof are provided. The methods comprise administering to the subject a therapeutically effective amount of an agonist of miR-34a-5p, an agonist of miR-99b-5p, or an antagonist of miR-96-5p.

In certain embodiments, the methods comprise administering to the subject a therapeutically effective amount of a miR-34a-5p mimic, a miR-99b-5p mimic, or a miR-96-5p antagomir. In certain embodiments, the miR-34a-5p mimic comprises a nucleotide sequence having at least about 80% identity to SEQ ID NO: 1. In certain embodiments, the miR-99b-5p mimic comprises having at least about 80% identity to SEQ ID NO: 2. In certain embodiments, the miR-96-5p antagomir comprises a nucleotide sequence having at least about 80% complementary to SEQ ID NO: 3.

In certain embodiments, administering an agonist of miR-34a-5p decreases expression of HIF1A, IGFBP2, and PIK3CB in the subject. In certain embodiments, administering an agonist of miR-99b-5p decreases expression of MTOR in the subject. In certain embodiments, administering an antagonist of miR-96-5p increases expression of MAPKAPK2 in the subject.

In certain embodiments, the subject has prostate cancer, breast cancer, lung cancer, or colon cancer. In certain other embodiments, the subject has an aggressive form of cancer (e.g., castration-resistant prostate cancer). In certain embodiments, the methods further comprise administering to the subject an anti-cancer therapy. Such anti-cancer therapies include, but are not limited to, chemotherapy (e.g., docetaxel), radiotherapy, immunotherapy, surgical resection, or gene therapy.

Methods of identifying a subject having or at risk of developing cancer are provided. In certain embodiments, the methods comprise assessing the level of miR-34a-5p, miR-99b-5p, or miR-96-5p in a sample from the subject. In certain embodiments, decreased miR-34a-5p, decreased miR-99b-5p, or increased miR-96-5p as compared to a control is indicative that the subject has or is at risk of developing prostate cancer, breast cancer, lung cancer, or colon cancer. In certain embodiments, decreased miR-34a-5p, decreased miR-99b-5p, or increased miR-96-5p as compared to a control is indicative that the subject has or is at risk of developing an aggressive form of cancer (e.g., castration-resistant prostate cancer). In certain embodiments, the methods comprise assessing the level of HIF1A, IGFBP2, PIK3CB, MTOR, or MAPKAPK2 in the sample.

In certain embodiments, the methods further comprise administering a therapeutically effective amount of an agonist of miR-34a-5p, an agonist of miR-99b-5p, or an antagonist of miR-96-5p to a subject identified as having or at risk of developing cancer. In certain embodiments, the methods further comprise administering an anti-cancer therapy to a subject identified as having or at risk of developing cancer.

Pharmaceutical compositions comprising a miR-34a-5p mimic, a miR-99b-5p mimic, or a miR-96-5p antagomir, and a pharmaceutically acceptable carrier are also provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows the mTOR signaling pathway is upregulated in African American prostate cancer specimens. MirPath v.3 was used to evaluate the impact of the 10 differentially expressed miRNAs (in African American prostate cancer vs. European American prostate cancer) in regulating the biological signaling pathways in African American prostate cancer compared to European American prostate cancer. Downregulated (green) and upregulated (red) miRNAs and mRNAs, in African American prostate cancer vs. European American prostate cancer, were indicated in mTOR signaling pathway. Three robust reciprocal miRNA-mRNA pairings, miR-34a-5p/IGFBP2 (down/up), miR-99b-5p/MTOR (down/up) and miR-34a-5p/HIF1A (down/up), were highlighted. Unpaired African American-depleted miRNAs (miR-125b-2-3p, miR-378a-5p and miR-34a-5p) and African American-enriched miRNAs (miR-130b-3p and miR-96-5p) were indicated adjacent to their predicted target genes. The genes highlighted with yellow are genes targeted by one African American-depleted/enriched miRNA. The genes highlighted with orange are genes targeted by at least two African American-depleted/enriched miRNAs. The genes highlighted with light green are genes not targeted by any African American-depleted/enriched miRNA.

FIG. 2 shows the VEGF signaling pathway is upregulated in African American prostate cancer specimens. Using mirPath v.3, VEGF signaling was identified as significant signaling pathway differentially regulated by African American depleted/enriched miRNAs (adjusted p-value=0.0155). Downregulated (green) and upregulated (red) miRNAs and mRNAs, in African American prostate cancer vs. European American prostate cancer, were mapped in VEGF signaling pathway. Two reciprocal miRNA-mRNA pairings, miR-34a-5p/PIK3CB (down/up) and miR-96-5p/MAPKAPK2 (up/down), were highlighted. Other unpaired downregulated and upregulated miRNAs (miR-125b-2-3p, miR-99b-5p and upregulated miR-130b-3p) were indicated next to their predicted target genes. The genes highlighted with yellow are genes targeted by one African American-depleted/enriched miRNA. The genes highlighted with orange are genes targeted by at least two African American-depleted/enriched miRNAs. The genes highlighted with light green are genes not targeted by any African American-depleted/enriched miRNA.

FIG. 3 shows RT-qPCR validation of miR-34a-5p, miR-99b-5p, and miR-96-5p in African American (AA) and European American (EA) prostate cancer specimens. Scatter dot plots with median values of log 2 values for miRNA expression levels from European American and African American prostate cancer were shown. Each dot represented the normalized miRNA expression level from an individual prostate cancer specimen. The relative miRNA expression levels were determined using endogenous miR-103a-3p for normalization. Significance (***p<0.001, and ****p<0.0001 in African American prostate cancer vs. European American prostate cancer) was determined based on student t-test.

FIG. 4A-B shows RT-qPCR validation of differentially expressed miRNAs and mRNAs in African American and European American prostate cancer cell line models. FIG. 4A shows RT-qPCR assays for examining expression level of miR-34a-5p, miR-99b-5p and miR-96-5p in cell lines derived from European American prostate cancer (LNCaP and PC-3) and African American prostate cancer (RC77 T/E and MDA PCa 2b). FIG. 4B shows RT-qPCR assays for examining expression level of PIK3CB, MTOR, HIF1A, IGFBP2 and MAPKAPK2 in African American and European American prostate cancer cell line models. Data were presented as mean±SEM of n=4-6 independent experiments, with technical triplicates for each independent experiment. The significance (*p<0.05 in African American prostate cancer cell line vs. LNCaP, and **p<0.05 in African American prostate cancer cell line vs. PC-3) was determined based on ANOVA with Tukey post hoc test.

FIG. 5A-C shows Western blot analysis of mTOR, PI3 KB, HIF1a, IGFBP2 and MAPKAPK2 in African American (AA) and European American (EA) prostate cancer cell line models. FIG. 5A shows representative Western blot images of mTOR, PI3 KB, HIF1a, IGFBP2, MAPKAPK2 and β-actin in European American prostate cancer (LNCaP, PC-3) and African American prostate cancer (RC77 T/E and MDA PCa 2b) cell lines. FIG. 5B shows normalized protein levels of mTOR, PI3 KB, HIF1a, IGFBP2 and MAPKAPK2 in European American prostate cancer and African American prostate cancer cell line models. The normalized protein level was measured by dividing intensity of the tested protein (mTOR, PI3 KB, HIF1a, IGFBP2 or MAPKAPK2) with intensity of endogenous protein β-actin. Data were presented as mean±SD of n=3-4 independent immunoblot experiments, and the significance (*p<0.05 in African American prostate cancer cell line vs. LNCaP, and **p<0.05 in African American prostate cancer vs. PC-3) was determined based on ANOVA with Tukey post hoc test. FIG. 5C shows phosphorylation states of mTOR and VEGF in European American prostate cancer (LNCaP and PC-3) and African American prostate cancer (RC77 T/E and MDA PCa 2b) cell models. The pmTOR/mTOR and pVEGFR/VEGFR ratios were significantly higher in MDA PCa 2b (a metastatic African American prostate cancer line) than in other three cell lines.

FIG. 6A-C shows transfection efficiencies of miR-34a-5p (FIG. 6A), miR-99b-5p (FIG. 6B), and miR-96-5p (FIG. 6C) in European American and African American prostate cancer cell lines. RT-qPCR assays revealed comparable transfection efficiencies of miR-34a-5p, miR-99b-5p, and miR-96-5p in LNCaP, PC-3, RC77 T/E, and MDA PCa 2b cells. The smaller fold changes between miR-96-5p and NS transfection in LNCaP and PC-3 simply reflect the fact that only very low levels (close to baseline) of miR-96-5p were detected in LNCaP and PC-3 before miR-96 transfection (as shown in FIG. 3A).

FIG. 7A-E shows modulation of miRNA expression affects the transcriptional regulation of its target genes in European American and African American prostate cancer cell lines. RT-qPCR analysis of MTOR, PIK3CB, HIF1A, IGFBP2 and MAPKAPK2 expression in European American and African American prostate cancer cell line models. Relative expression levels of MTOR, PIK3CB, HIF1A, IGFBP2 and MAPKAPK2 were shown in European American prostate cancer (LNCaP and PC-3) and African American prostate cancer (RC77 T/E and MDA PCa 2b) cell lines transfected with nonsense scrambled miRNA (NS), miRNA mimic or antagomir (miR-34a-5p mimic, miR-99b-5p mimic, or miR-96-5p antagomir). Data were presented as mean±SEM of n=3-4 independent RT-qPCR experiments, with technical triplicates for each independent experiment. The significance (*p<0.05 in miRNA mimic or antagomir vs. NS) was determined based on student's t-test.

FIG. 8A-D shows Western blot analysis of mTOR, PI3 KB, HIF1a, IGFBP2 and MAPKAPK2 in European American and African American prostate cancer cell line models transfected with miRNA mimics or antagomir. Protein levels of mTOR, PI3 KB, HIF1a, IGFBP2, MAPKAPK2 and β-actin were shown in European American prostate cancer (LNCaP and PC-3) and African American prostate cancer (RC77 T/E and MDA PCa 2b) cell lines transfected with nonsense scrambled miRNA (NS), miR-34a-5p mimic, miR-99b-5p mimic, or miR-96-5p antagomir. Representative Western blot images were selected from 3-4 independent immunoblot assays with consistent results.

FIG. 9A-D shows overexpressing African American-depleted miRNAs or suppressing African American-enrich miRNA causes inhibition of cell proliferation in European American and African American prostate cancer cell lines. BrdU-labeling assays were performed after prostate cancer cell lines were transfected with NS, miR-34a-5p mimic, miR-99b-5p mimic or miR-96-5p antagomir for 48 h. Data were plotted as mean±SEM of n=3-4 independent assays, with 3-4 technical replicates for each independent assay. The significance (*p<0.05 in miRNA mimic/antagomir vs. NS) was determined based on ANOVA with Dunnett post hoc test.

FIG. 10A-D shows overexpression of miR-34a-5p mimic, miR-99b-5p mimic or miR-96-5p antagomir enhances docetaxel-induced cytotoxicity in European American prostate cancer (FIG. 10A, FIG. 10B) and African American prostate cancer (FIG. 10C, FIG. 10D) cell lines. Apoptosis activity was assessed by measuring caspase-3/7 activity using the Apo-ONE Kit, and the data were normalized to caspase-3/7 level of vehicle-treated NS control. Data were plotted as mean±SEM for n of 3-4 independent experiments, with technical triplicates for each independent experiment. The significance (*p<0.05 in miRNA mimic or antagomir transfection plus vehicle treatment vs. NS plus vehicle treatment, and **p<0.05 in miRNA mimic or antagomir transfection plus docetaxel treatment to miRNA mimic or antagomir transfection with vehicle treatment) was determined using ANOVA with Tukey post hoc test.

FIG. 11A-C shows IHC staining assays for examining mTOR and AMACR protein levels in prostate cancer patient specimens. FIG. 11A shows quantification of IHC staining signals from mTOR in European American and African American prostate cancer specimens (left panel) and representative IHC images from European American and African American prostate cancer specimens (right panel). NEA: adjacent normal specimen derived from European American patient, TEA: tumorous European American specimen, NAA: adjacent normal specimen from African American prostate cancer, TEA: tumorous African American specimen. Higher mTOR intensities were shown in African American prostate cancer vs. European American prostate cancer with comparable Gleason scores (3+4, 4+4, and 5+4). *** Significance (p-value<0.001, comparing mTOR staining intensities in African American prostate cancer vs. European American prostate cancer specimens) was determined based on ANOVA with Tukey post-hoc test. FIG. 11B shows quantification (left panel) and representative IHC images of mTOR and AMACR staining in prostate cancer patient specimens (right panel), mTOR and AMACR staining intensities were measured in normal prostate tissues and prostate cancer specimens in the TMAs. GS: Gleason Score. ***Significance (p-value<0.0001, comparing AMACR or mTOR staining intensities in prostate cancer vs. normal tissues) was determined based on paired 1-test. FIG. 11C shows higher frequency of nuclear mTOR signals was detected in African American prostate cancer specimens (left panel). Representative IHC staining revealed both nuclear (Nuc) and cytoplasmic (Cy) mTOR expression in European American prostate cancer (TEA) and African American prostate cancer (TAA) specimens. Note that high-level nuclear (nearly exclusive) mTOR signals were detected in TAA #2 and #3 samples (right panel). Percentage of nuclear mTOR-positive TEA and TAA samples were calculated based on the equation of (number of nuclear mTOR-positive specimens/number of mTOR-positive specimens)×100% in TEAs and TAAs, respectively.

FIG. 12-C shows immunofluorescence staining demonstrated higher expression levels of mTOR, nuclear mTOR, and nuclear pmTOR in African American prostate cancer when compared to European American prostate cancer cells. FIG. 12A shows immunofluorescence showing mTOR (green fluorescence) and pmTOR (red fluorescence) signals in European American prostate cancer cell lines (22Rv1, LNCaP and PC-3), and African American prostate cancer cell lines (RC77 T/E and MDA PCa 2b). Nuclei were visualized by counterstaining with DAPI (blue fluorescence). Merged images were achieved by overlaying DAPI, mTOR and pmTOR signals to identify colocalization (yellow) of mTOR and pmTOR in nuclei. Fluorescent image capturing and analysis were performed using CellScans software V1.18. FIG. 12B shows total mTOR and nuclear mTOR signals in European American and African American prostate cancer cell lines. Fluorescence mTOR- or pmTOR-positive cells (%) were determined based on (number of mTOR or pmTOR-positive cells/number of DAPI-positive cells)×100%.

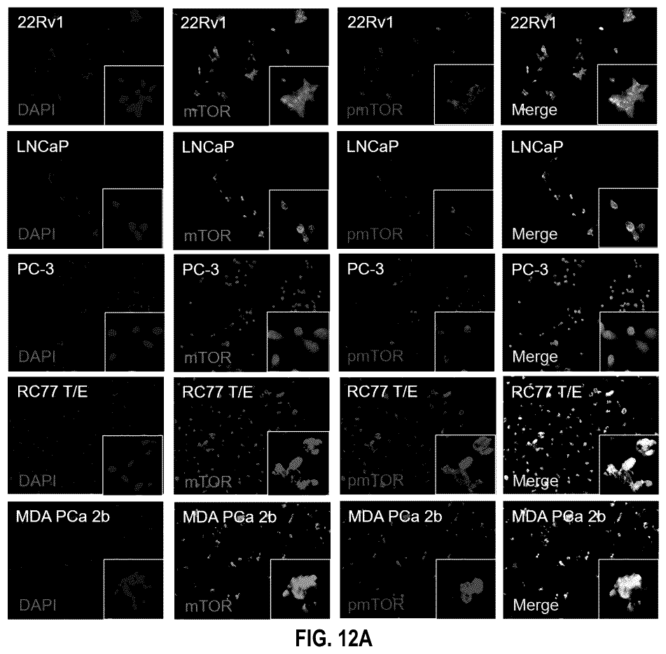

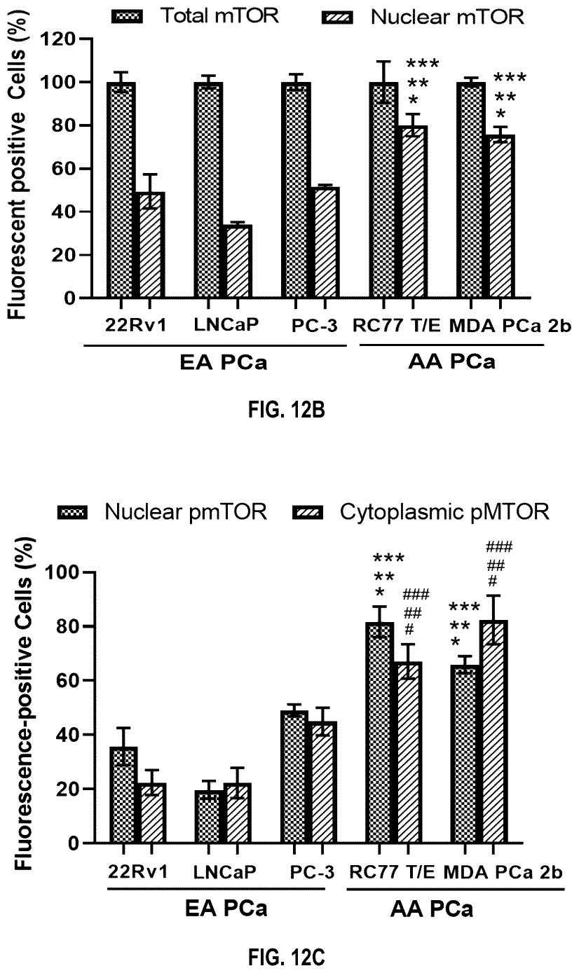

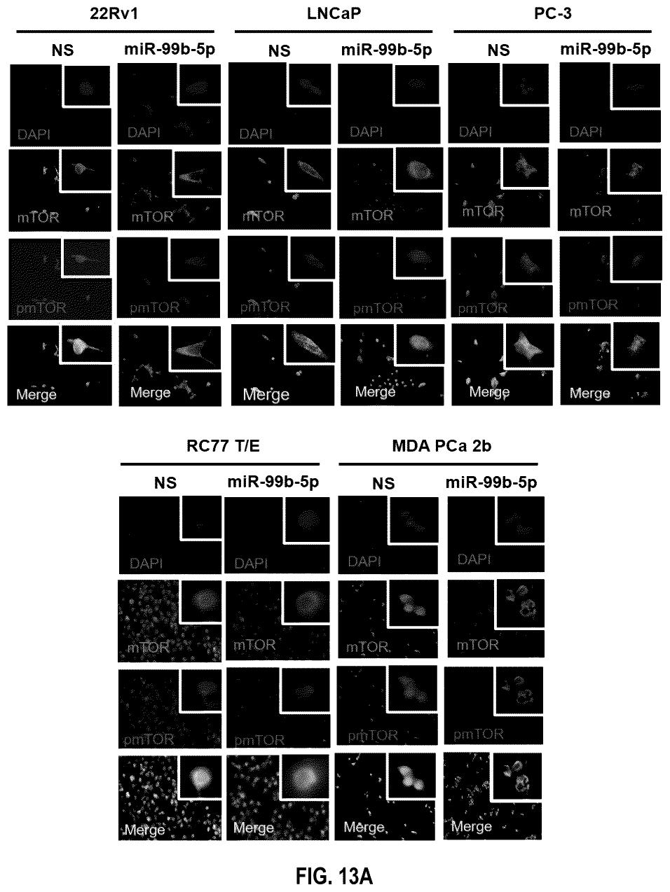

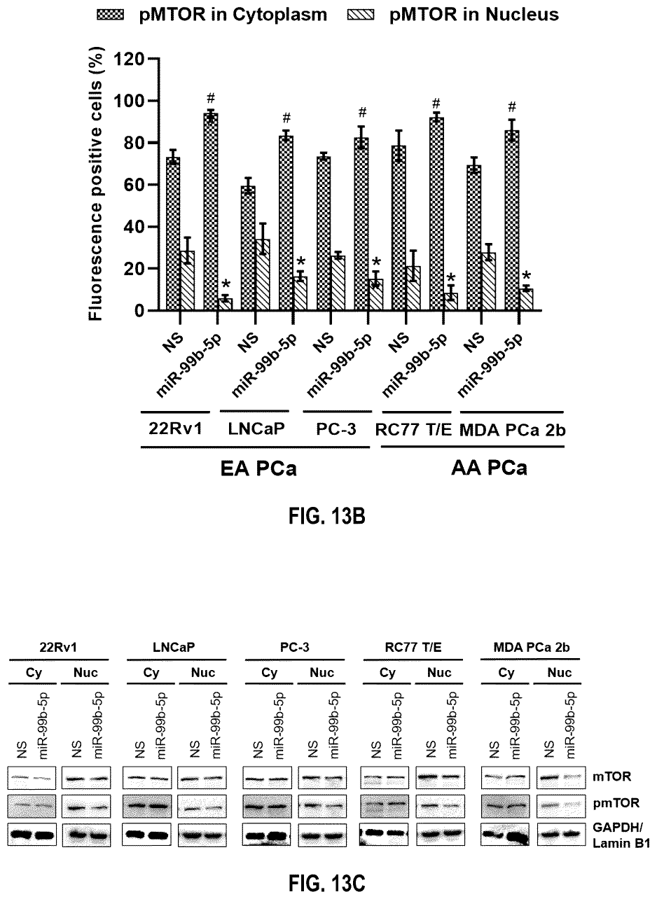

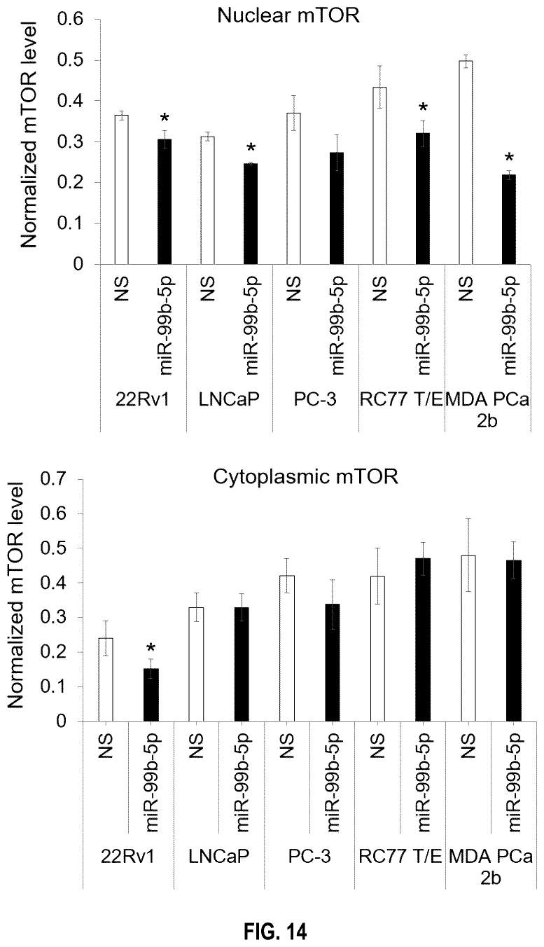

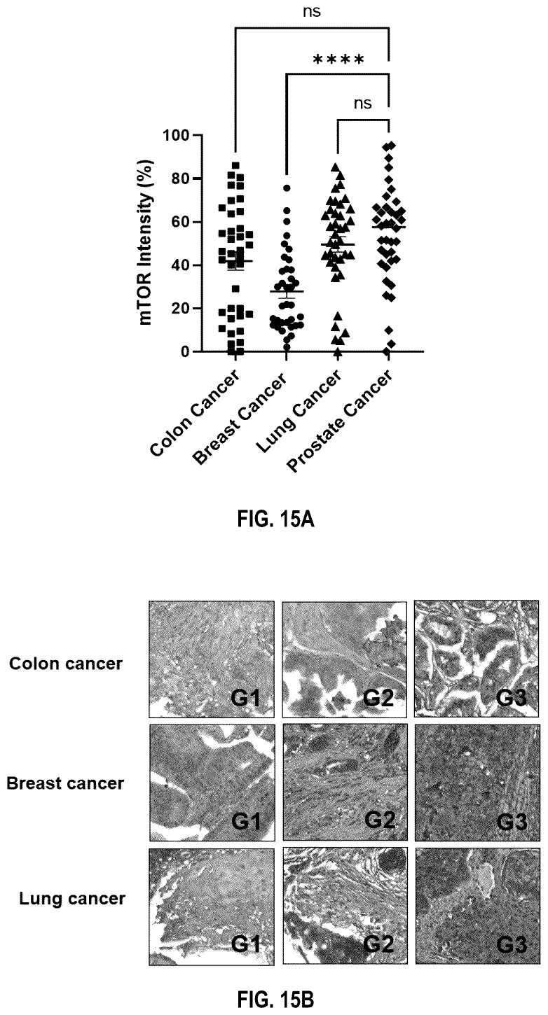

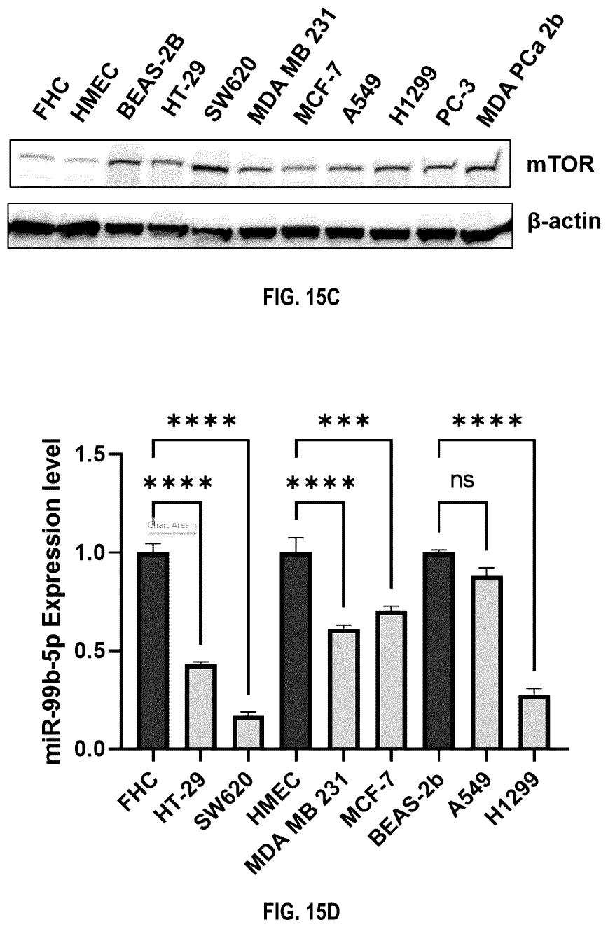

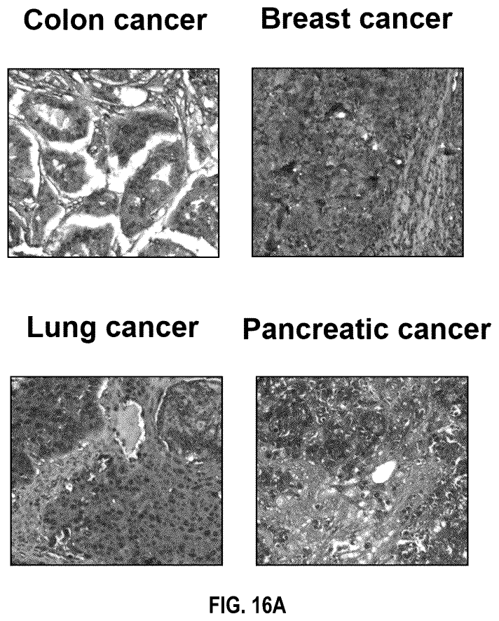

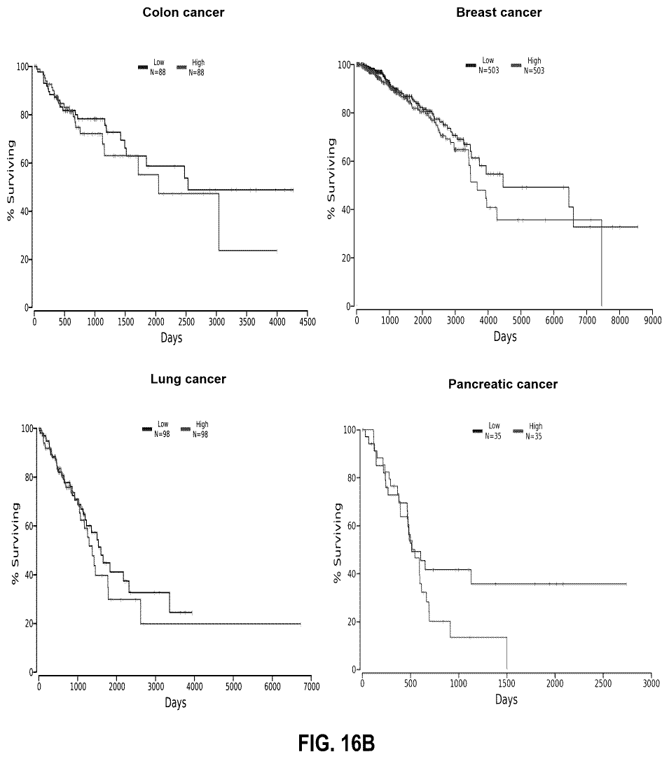

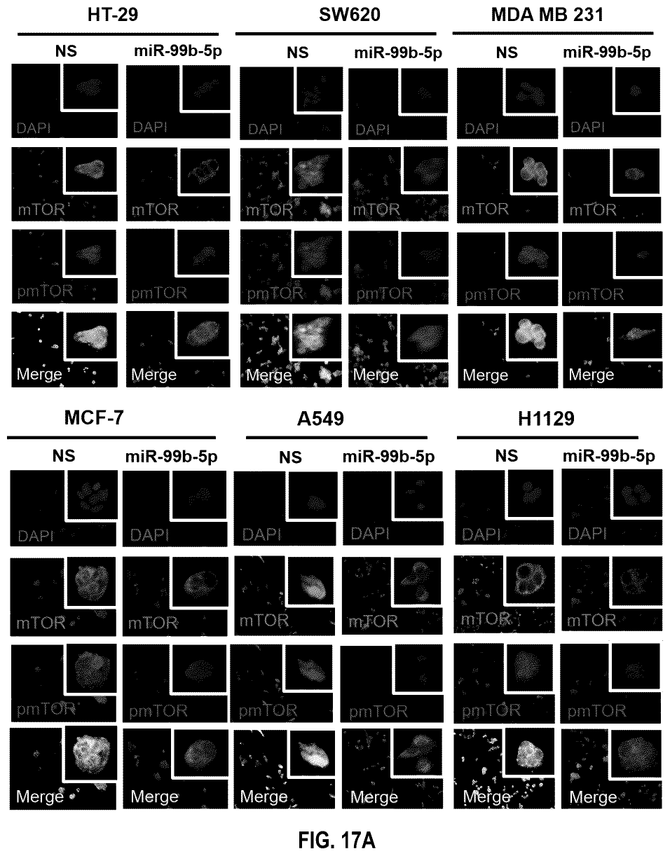

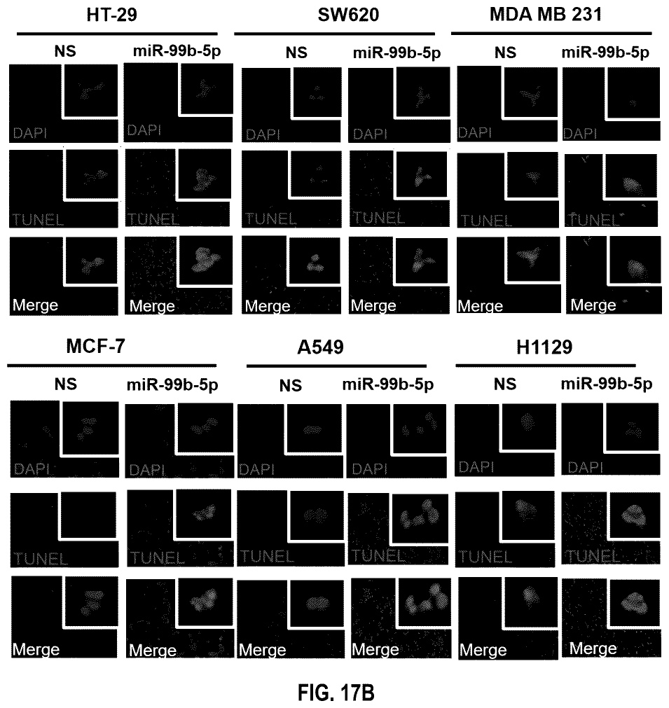

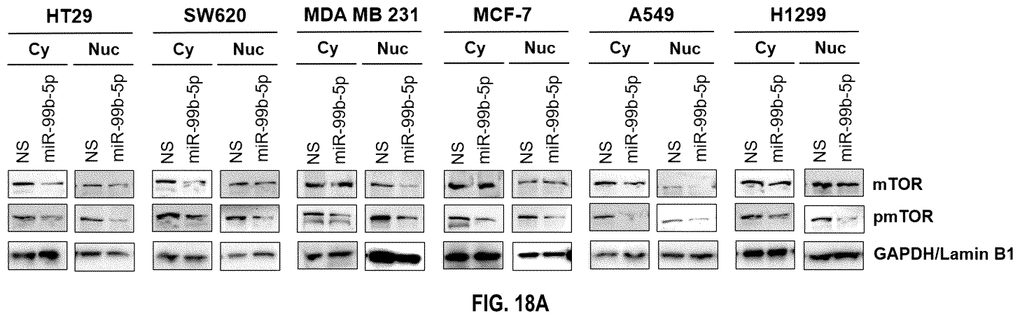

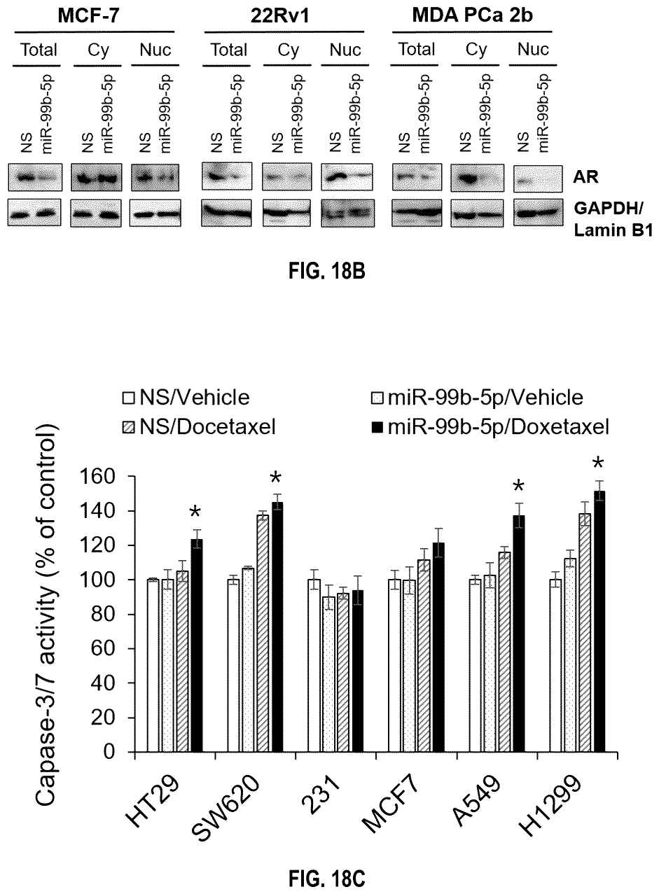

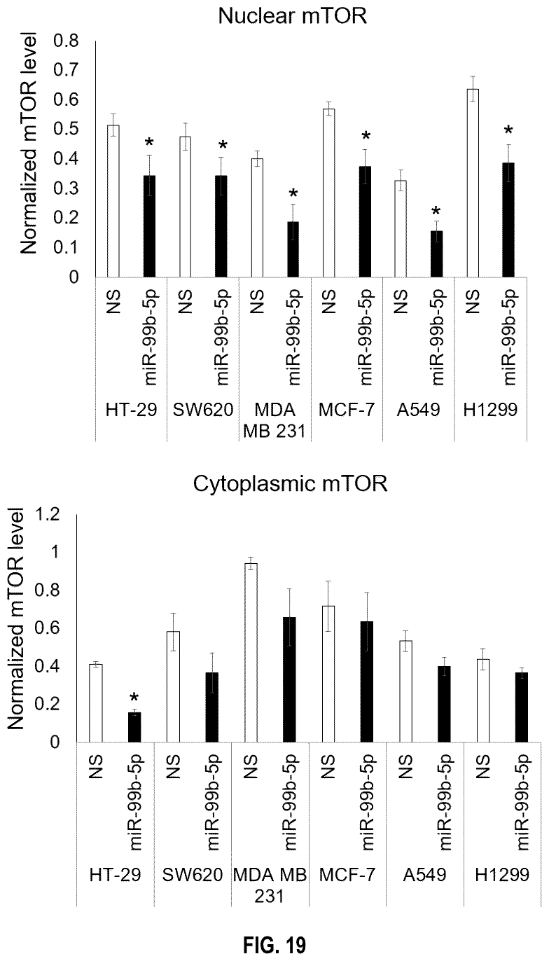

Significance was determined based on ANOVA with Tukey's post-hoc test (*p-value<0.001 in 22Rv1 vs. RC77 T/E or vs. MDA PCa 2b, **p-value<0.001 in LNCaP vs. RC77 T/E or vs. MDA PCa 2b, and ***p-value<0.001 in PC-3 vs. RC77 T/E or vs. MDA PCa 2b). FIG. 12C shows distribution of cytoplasmic and nuclear pmTOR in European American and African American prostate cancer cell lines. Significant difference of nuclear mTOR (*p-value<0.001 in 22Rv1 vs. RC77 T/E or vs. MDA PCa 2b, **p-value<0.001 in LNCaP vs. RC77 T/E or vs. MDA PCa 2b, and ***p-value<0.001 in PC-3 vs. RC77 T/E or vs. MDA PCa 2b) and nuclear pmTOR (^{#}p-value<0.001 in 22Rv1 vs. RC77 T/E or vs. MDA PCa 2b, ^{##}p-value<0.001 in LNCaP vs. RC77 T/E or vs. MDA PCa 2b, and ^{## #}p-value<0.001 in PC-3 vs. RC77 T/E or vs. MDA PCa 2b) were shown in African American prostate cancer vs. European American prostate cancer. The statistics were determined based on ANOVA with Tukey's post-hoc test, and each value was represented as mean±SEM (n=6).

FIG. 13A-C shows transfection of miR-99b-5p mimic attenuates mTOR and pmTOR expressions and blocks the translocation of pmTOR to the nuclei. FIG. 13A shows immunofluorescence showing cytoplasmic and nuclear localizations of mTOR (green fluorescence) and pmTOR (red fluorescence) in European American prostate cancer cell lines (22Rv1, LNCaP and PC-3) and African American prostate cancer cell lines (RC77 T/E and MDA PCa 2b) transfected with NS or miR-99b-5p mimic. The nuclei were visualized by counterstaining with DAPI. FIG. 13B shows quantification analysis of cytoplasmic and nuclear distribution in European American and African American prostate cancer cells. Significant increase in cytoplasmic pmTOR (#p-value<0.01 using ANOVA with Tukey's post-hoc test) and a significant decrease in nuclear pmTOR (*p-value using ANOVA with Tukey's post-hoc test) was observed in the miR-99b-5p mimic vs. NS transfected cells. Each value was represented as mean±SEM (n=6). FIG. 13C shows western blot analysis of mTOR and pmTOR in cytoplasmic and nuclear fractions of European American and African American prostate cancer cell lines transfected with NS or the miR-99b-5p mimic. The representative images shown here were selected from 3-4 independent Western blot results. GAPDH and Lamin B1 were used as endogenous controls for cytoplasmic (Cy) and nuclear (Nu) proteins, respectively.

FIG. 14 shows quantification of nuclear and cytoplasmic mTOR protein levels in prostate cancer cells. The nuclear and cytoplasmic proteins extracted from the prostate cancer cells (22Rv1, LNCaP, PC-3, RC77 T/E, and MDA PCa 2b) were subjects to the western blot analysis. Quantification of nuclear and cytoplasmic mTOR levels were determined by normalization of mTOR signals with Lamin B1 and GAPDH, respectively. Each value was represented as mean±SD, based on 3-4 western blot images. Significant difference (p-value<0.05, based on ANOVA with Tukey's post-hoc test) of normalized mTOR levels were shown between miR-99b-5p mimic vs. NS transfected prostate cancer cells.

FIG. 15A-D shows mTOR and miR-99b-5p expression profiles in colon, breast, and lung cancer cell models. FIG. 15A shows quantification of mTOR intensities in colon, breast, lung, and prostate cancer specimens using IHC staining assay. IHC staining assay was applied to examine mTOR protein levels in various solid tumor patient specimens on a TMA slide. Significantly different mTOR intensities were identified in prostate cancer vs. breast cancer specimens (****p-value<0.0001, based on ANOVA with Dunnett's post-hoc test). No significant difference (ns) in mTOR intensities was found in colon cancer vs. prostate cancer, or lung cancer vs. prostate cancer specimens. FIG. 15B shows representative IHC staining images of the mTOR protein in colon, breast, and lung cancer specimens. Apparently, the expression levels of mTOR were gradually increased from low-to high-grade cancer samples. G1: grade 1 tumor, G2: grade 2 tumor, and G3: grade 3 tumor. FIG. 15C shows western blot analysis of mTOR protein levels in a panel of normal and cancer cell lines. Representative Western blot analysis of mTOR protein levels from total protein extracts of colon cancer (HT-29, SW620), breast cancer (MDA MB 231, MCF-7), lung cancer (A549, H1299), and prostate cancer (PC-3, MDA PCa 2b) cell lines and control cell lines (FHC, HMEC and BEAS-2B from normal colon, breast and lung tissues, respectively). FIG. 15D shows RT-qPCR assays showed downregulation of miR-99b-5p in colon, breast and lung cancer cell lines, compared to their normal controls. RNA samples isolated from FHC, HMEC, HT-29, SW620, MDA MB 231, MCF-7, BEAS-2B, A549, and H1299 were subjected to RT-qPCR assays of miR-99b-5p. Significantly different miR-99b-5p expression levels (****p-value<0.0001 and ***p-value<0.001, based on ANOVA with Tukey's post-hoc test) were shown in cancer cell lines vs. normal controls (except A549 vs. BEAS-2B). ns: not significant. Each value was represented as mean±SEM, obtained from three independent cDNA samples with duplicate or triplicate qPCR reactions. ns: not significant.

FIG. 16A shows IHC staining of mTOR protein on a TMA containing colon, breast, lung, and pancreatic cancer specimens. The representative IHC images of mTOR staining from the indicated 4 cancer specimens were presented. FIG. 16B shows survival curves for cancer patients with high-level mTOR (red curve) and low-level mTOR (blue curve) expression levels. The mTOR expression data were obtained from TCGA-RNAseq database, and OncoLnc program (oncolnc.org) was used to plot the survival curves.

FIG. 17A-B shows overexpression of miR-99b-5p changes the subcellular distribution of mTOR and pmTOR and initiates cell apoptosis. FIG. 17A shows immunofluorescence assays were used to visualize the subcellular localization of mTOR (green fluorescence) and pmTOR (green fluorescence) signals in cancer cell lines (HT-29, SW620, MDA MB 231, MCF-7, A549, and H1299) transfected with NS or miR-99b-5p mimic. Nuclei were visualized by counterstaining with DAPI. FIG. 17B shows TUNEL assays were used to visualize the DNA damages created during apoptotic events in the cancer cell lines upon miR-99b-5p and NS transfections. Apoptotic events were detected based on the DNA damages (visualized as red fluorescent spot signals, TUNEL panel) in the nuclei (blue, DAPI panel). The red/purple signals shown by overlaying DAPI and TUNEL signals (Merge panel) indicated apoptotic activities (DNA damages occurring in the nuclei) in the cancer cells.

FIG. 18A-C shows overexpression of miR-99b-5p inhibits mTOR and pmTOR expression and nuclear translocation, and sensitizes the docetaxel-induced cytotoxicity in cancer cells. FIG. 18A shows mTOR and pmTOR protein levels in cytoplasmic and nuclear fractions. GAPDH and Lamin B1 were used as endogenous controls for cytoplasmic (Cy) and nuclear (Nu) proteins, respectively. FIG. 18B shows AR levels in total cell lysates (Total), cytoplasm (Cy), and nuclei (Nu) from the cancer cells (MCF-7, 22Rv1, and MDA PCa 2b) transfected with NS or miR-99b-5p mimic. The representative images were selected from 3-4 independent Western blot results. GAPDH was used as endogenous control for total and cytoplasmic proteins, and Lamin B1 was used as endogenous protein control for the nuclear proteins. FIG. 18C shows apoptosis assays were performed in the various cancer cell lines transfected with NS or miR-99b-5p mimic in the absence or presence of 11 nM docetaxel. Significantly different apoptosis capacity (*p-value<0.05, in miR-99b-5p transfected cells treated with docetaxel vs. vehicle) were determined based on ANOVA with Tukey's post-hoc test. Each value was represented as mean±SD (n=3-4); 231: MDA MB 231.

FIG. 19 shows quantification of nuclear and cytoplasmic mTOR protein levels in colon, breast and lung cancers. The nuclear and cytoplasmic proteins extracted from the cancer cells (HT-27, SW620, MDA MB 231, MCF-7, A549, and H1299) were subjects to the western blot analysis. Quantification of nuclear and cytoplasmic mTOR levels were determined by normalization of mTOR signals with Lamin B1 and GAPDH signals, respectively. Each value was represented as mean±SD, based on 3-4 western blot images. Significant difference (p-value<0.05, based on ANOVA with Tukey's post-hoc test) of normalized mTOR levels were shown between miR-99b-5p mimic vs. NS transfected cancer cells.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the terms “antagonist” and “inhibitor” are used interchangeably to refer to molecules (e.g., nucleic acids) that decrease the expression or function of a miRNA.

As used herein, the term “agonist” refers to molecules (e.g., nucleic acids) that mimic or increase the expression or function of a miRNA.

### MicroRNAs (Mirnas)

Human miRNAs are ˜22 nucleotide (nt) non-coding RNAs, involved in the regulation of post-transcriptional gene expression profile. It is estimated that more than 60% of human protein coding genes are regulated by the miRNAs. MiRNAs play critical roles in biological processes such as cell proliferation, cell growth, intracellular signaling, cell differentiation, cell apoptosis, cellular metabolism and carcinogenesis. They usually bind to the 3′-untranslated region (3′-UTR) of the target mRNAs, destabilizing the mRNA and control protein production through translational silencing. The genes encoding miRNAs locate in exonic or intronic regions and are transcribed by RNA polymerase II, resulting in pri-miRNAs. The pri-miRNAs are further processed by Drosha complex to form the ˜70 nt stem-loop pre-miRNAs. Exportin-5 and Ran-GTP transports pre-miRNAs from the nucleus to cytoplasm, where Dicer further processes pre-miRNAs into 20-25 nucleotide long mature miRNA-miRNA duplexes. These mature miRNAs are then loaded onto Argonaute 2 protein (AGO2) and RNA-induced silencing complex (RISC) to achieve site-specific cleavage/degradation or translational inhibition of the target mRNAs.

The present disclosure involves, in part, the discovery that several miRNAs (miR-34a-5p, miR-99b-5p, miR-96-5p) are dysregulated in certain types of cancer. The sequence for miR-34a-5p is provided as SEQ ID NO: 1 (UGGCAGUGUCUUAGCUGGUUGU), miRBase accession number MIMAT0000255. The sequence for miR-99b-5p is provided as SEQ ID NO: 2 (CACCCGUAGAACCGACCUUGCG), miRBase accession number MIMAT0000689. The sequence for miR-96-5p is provided as SEQ ID NO: 3 (UUUGGCACUAGCACAUUUUUGCU), miRBase accession number MIMAT0000095.

Agonists and Antagonists of miRNAs

Agonists of a miRNA (e.g., miR-34a-5p or miR-99b-5p) will generally take one of three forms. First, there is miRNA itself. Such molecules may be delivered to target cells, for example, by injection or infusion, optionally in a delivery vehicle such as a lipid, such as a liposome or lipid emulsion. Second, one may use expression vectors that drive or alter the expression of the miRNA. The composition and construction of various expression vectors is described elsewhere herein. Third, one may use agents distinct from the miRNA that act to up-regulate, stabilize or otherwise enhance the activity of the miRNA, including small molecules. Such molecules include “mimetics”, molecules which mimic the function, and possibly form of a miRNA, but are distinct in chemical structure.

Antagonism of a miRNA (e.g., miR-96-5p) may, for example, be achieved by “antagomirs”. Initially described by Krützfeldt and colleagues (Krützfeldt et al., Nature, 438:685-689, 2005), antagomirs are single-stranded, chemically-modified ribonucleotides that are at least partially complementary to the miRNA sequence. Antagomirs may comprise one or more modified nucleotides, such as 2′-O-methyl-sugar modifications. In certain embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a cholesterol moiety at its 3′ end. Antagomirs suitable for inhibiting miRNAs may be about 14 to about 50 nucleotides in length, about 14 to about 30 nucleotides in length, and 14 to about 25 nucleotides in length. “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In certain embodiments, the antagomir may be substantially complementary to a mature miRNA sequence, that is at least about 95%, 96%, 97%, 98%, or 99% complementary to a target polynucleotide sequence. In certain embodiments, the antagomirs are 100% complementary to the mature miRNA sequence.

Inhibition of a miRNA (e.g., miR-96-5p) may also be achieved by administering antisense oligonucleotides. The antisense oligonucleotides may be ribonucleotides or deoxyribonucleotides. Preferably, the antisense oligonucleotides have at least one chemical modification. Antisense oligonucleotides may be comprised of one or more “locked nucleic acids.” “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g., 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In certain embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the disclosure. Particular antisense oligonucleotides useful for inhibiting the activity of microRNAs are about 19 to about 25 nucleotides in length. Antisense oligonucleotides may comprise a sequence that is at least partially complementary to a mature miRNA sequence, e.g., at least about 75%, 80%, 85%, 90%, 95%, 96%, 97%, 98%, or 99% complementary to a mature miRNA sequence. In certain embodiments, the antisense oligonucleotide may be substantially complementary to a mature miRNA sequence, that is at least about 95%, 96%, 97%, 98%, or 99% complementary to a target polynucleotide sequence. In one embodiment, the antisense oligonucleotide comprises a sequence that is 100% complementary to a mature miRNA sequence.

Another approach for inhibiting the function of a miRNA (e.g., miR-96-5p) is administering an inhibitory RNA molecule having at least partial sequence identity to the mature miRNA sequence. The inhibitory RNA molecule may be a double-stranded, small interfering RNA (siRNA) or a short hairpin RNA molecule (shRNA) comprising a stem-loop structure. The double-stranded regions of the inhibitory RNA molecule may comprise a sequence that is at least partially identical, e.g., about 75%, 80%, 85%, 90%, 95%, 96%, 97%, 98%, or 99% identical, to the mature miRNA sequence. In certain embodiments, the double-stranded regions of the inhibitory RNA comprise a sequence that is at least substantially identical to the mature miRNA sequence. “Substantially identical” refers to a sequence that is at least about 95%, 96%, 97%, 98%, or 99% identical to a target polynucleotide sequence. In certain embodiments, the double-stranded regions of the inhibitory RNA molecule may contain 100% identity to the target miRNA sequence.

In certain embodiments, antagonists of a miRNA (e.g., miR-96-5p) may be inhibitory RNA molecules, such as ribozymes, siRNAs, or shRNAs. In certain embodiments, an inhibitor of miR-96-5p is an inhibitory RNA molecule comprising a double-stranded region, wherein the double-stranded region comprises a sequence having 100% identity to the mature miRNA sequence. In certain embodiments, inhibitors are inhibitory RNA molecules which comprise a double-stranded region, wherein said double-stranded region comprises a sequence of at least about 75%, 80%, 85%, 90%, 95%, 96%, 97%, 98%, or 99% identity to the mature miRNA sequence.

### Methods Of Treatment

In certain embodiments, the disclosure provides compositions and methods for the treatment of cancer, including prostate cancer, breast cancer, lung cancer, and colon cancer as well as aggressive forms of cancer (e.g., castration-resistant prostate cancer). In certain embodiments, the disclosure provides a method of treating cancer comprising administering to the subject a therapeutically effective amount of an agonist of miR-34a-5p, an agonist of miR-99b-5p, or an antagonist of miR-96-5p. This treatment may be further combined with additional cancer treatments. One of skill in the art will be aware of many treatments that may be combined with the methods of the present disclosure, some but not all of which are described below.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

Formulations for delivery of the miRNA agonist or antagonist (e.g., miRNA mimic or antagomir) are selected based on the route of administration and purpose including, but not limited to, liposomal formulations and classic pharmaceutical preparations (discussed below).

Non-limiting examples of agents suitable for formulation with the miRNAs include P-glycoprotein inhibitors (such as PluronicP85), which can enhance entry of drugs into the CNS (Jolliet-Riant and Tillement, Fundam. Clin. Pharmacol., 13:16, 1999), biodegradable polymers, such as poly(DL-lactide-coglycolide) microspheres for sustained release delivery. Other non-limiting examples of delivery strategies for miRNAs include material described in Boado et al. (J. Pharm. Sci., 87(11): 1308-15, 1998), Tyler et al. (Am. J. Physiol., 277(6 Pt 1): L1199-204, 1999; Proc. Natl. Acad. Sci. USA, 8; 96(12):7053-8, 1999); Pardridge et al. (Proc. Natl. Acad. Sci. USA, 92:5592, 1995); Boado (Adv. Drug Delivery Rev., 15:73, 1995); Aldrian-Herrada et al. (Nucleic Acids Res., 26:4910-16, 1998).

The disclosure also includes the use of a composition that includes surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., Chem. Rev. 95:2601, 1995; Ishiwata et al., Chem. Phare. Bull., 43:1005, 1995).

Such liposomes have been shown to accumulate selectively in tumors, presumably by extravasation and capture in the neovascularized target tissues (Lasic et al., Science, 267:1275, 1995; Oku et al., Biochim. Biophys. Acta, 1238:86, 1995). The long-circulating liposomes enhance the pharmacokinetics and pharmacodynamics of DNA and RNA, particularly compared to conventional cationic liposomes which are known to accumulate in tissues of the MPS (Liu et al., Cancer Res., 55(14):3117-3122, 1995; PCT Publication No. WO 96/10391; PCT Publication No. WO 96/10390; PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

### Genetic Delivery

The use of expression constructs encoding miRNAs are also contemplated. The construction and structure of viral vectors is discussed below. Administration protocols would generally involve intratumoral, local or regional (to the tumor) administration, as well as systemic administration in appropriate clinical situations.

### Formulations and Routes for Administration to Patients

In certain embodiments, the disclosure provides a method of treating cancer comprising providing to a subject an effective amount of a miRNA agonist or antagonist (e.g., a miRNA mimic or antagomir). Where clinical applications are contemplated, it will be necessary to prepare pharmaceutical compositions in a form appropriate for the intended application.

Generally, this will entail preparing compositions that are essentially free of pyrogens, as well as other impurities that could be harmful to humans or animals.

One will generally desire to employ appropriate salts and buffers to render delivery nucleic acids stable and allow for uptake by target cells. Buffers may be employed when nucleic acids are introduced into a patient. In certain embodiments, Compositions of the present disclosure comprise an effective amount of a miRNA agonist or antagonist (e.g., a miRNA mimic or antagomir), dissolved or dispersed in a pharmaceutically acceptable carrier.

The phrase “pharmaceutically or pharmacologically acceptable” refer to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The active compositions of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present disclosure may be formulated in a neutral or salt form. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dosc(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

In certain embodiments, the subject can be a subject who is known or suspected of being free of a particular disease or health-related condition at the time the relevant preventive agent is administered. The subject, for example, can be a subject with no known disease or health-related condition (i.e., a healthy subject).

In certain embodiments, methods include identifying a subject in need of treatment. A patient may be identified, for example, based on taking a patient history or based on findings on clinical examination.

### Cancer Combination Treatments

In certain embodiments, the method further comprises treating a subject with cancer with a conventional cancer treatment. One goal of current cancer research is to find ways to improve the efficacy of chemo- and radiotherapy, such as by combining traditional therapies with other anti-cancer treatments. In the context of the present disclosure, it is contemplated that this treatment could be, but is not limited to, chemotherapeutic, radiation, or other therapeutic intervention. It also is conceivable that more than one administration of the treatment will be desired.

1. Chemotherapy

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosourcas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trictylenephosphoramide, tricthiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); cleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calichcamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabinc, 6-mercaptopurine, thiamiprinc, thioguanine; pyrimidine analogs such as ancitabine, azacitidine, 6-azauridine, carmofur, cytarabine, didcoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotricthylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, ctoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

### 2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, cither directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with the miRNA agonists or antagonists discussed above. In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, crb B and p155. An alternative aspect of immunotherapy is to combine anticancer effects with immune stimulatory effects. Immune stimulating molecules also exist including: cytokines such as IL-2, IL-4, IL-12, GM-CSF, Y-IFN, chemokines such as MIP-1, MCP-1, IL-8 and growth factors such as FLT3 ligand. Combining immune stimulating molecules, either as proteins or using gene delivery in combination with a tumor suppressor has been shown to enhance antitumor effects (Ju et al., Gene Ther., 7(19): 1672-1679, 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, Infection Immun., 66(11):5329-5336, 1998; Christodoulides et al., Microbiology, 144 (Pt 11):3027-3037, 1998), cytokine therapy, e.g., interferons α, β, and γ; IL-1, GM-CSF and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998) gene therapy, e.g., TNF, IL-1, IL-2, p53 (Qin et al., Proc. Natl. Acad. Sci. USA, 95(24): 14411-14416, 1998; Austin-Ward and Villaseca, Revista Medica de Chile, 126(7):838-845, 1998; U.S. Pat. Nos. 5,830,880 and 5,846,945) and monoclonal antibodies, e.g., anti-ganglioside GM2, anti-HER-2, anti-p185 (Pictras et al., Oncogene, 17(17):2235-2249, 1998; Hanibuchi et al., Int. J. Cancer, 78(4):480-485, 1998; U.S. Pat. No. 5,824,311). It is contemplated that one or more anti-cancer therapies may be employed with the miRNA agonist or antagonist therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, Intern. Rev. Immunol., 7: 303-329, 1991; Morton et al., Arch. Surg., 127:392-399, 1992; Mitchell et al., J. Clin. Oncol., 8(5):856-869, 1990; Mitchell et al., Ann. NY Acad. Sci., 690:153-166, 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., N. Engl. J. Med., 319:1676, 1988; Rosenberg et al., Ann. Surg. 210(4):474-548, 198).

### 4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the secondary treatment is a gene therapy in which a therapeutic polynucleotide is administered before, after, or at the same time as a miRNA, or a mimic or antagomir thereof, is administered. Delivery of a miRNA, or a mimic or antagomir thereof, in conjunction with a vector encoding one of the following gene products may have a combined anti-hyperproliferative effect on target tissues. A variety of proteins are encompassed within the disclosure, some of which are described below.

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present disclosure, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., Nature, 366:704-707, 1993; Serrano et al., Science, 267(5195): 249-252, 1995). Since the p16^{INK4} protein is a CDK4 inhibitor, deletion of this gene may increase the activity of CDK4, resulting in hyperphosphorylation of the Rb protein. p16 also is known to regulate the function of CDK6. p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines.

Other genes that may be employed according to the present disclosure include Rb, APC, DCC, NF-1, NF-2, WT-1, MEN-I, MEN-II, zac1, p73, VHL, MMACI, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, crb, fms, trk, ret, gsp, hst, abl, ElA, p300, genes involved in angiogenesis (e.g., VEGF, FGF, thrombospondin, BAI-1, GDAIF, or their receptors) and MCC.

Apoptosis, or programmed cell death, is an essential process for normal embryonic development, maintaining homeostasis in adult tissues, and suppressing carcinogenesis (Kerr et al., Br. J. Cancer, 26(4):239-257, 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., Cell, 41(3):899-906, 1985; Cleary and Sklar, Proc. Natl. Acad. Sci. USA, 82(21): 7439-7443, 1985; Cleary et al., J. Exp. Med., 164(1):315-320, 1986; Tsujimoto et al., Nature, 315:340-343, 1985; Tsujimoto and Croce, Proc. Natl. Acad. Sci. USA, 83(14):5214-5218, 1986). The evolutionarily conserved Bcl-2 protein now is recognized to be a member of a family of related proteins, which can be categorized as death agonists or death antagonists.

Subsequent to its discovery, it was shown that Bel-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclxL, Bclw, Bcls, Mcl-1, A1, Bfl-1) or counteract Bcl-2 function and promote cell death (e.g., Bax, Bak, Bik, Bim, Bid, Bad, Harakiri).

6. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In certain embodiments, cytostatic or differentiation agents can be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches.

### Dosage

Certain factors may influence the dosage required to effectively treat a subject, including but not limited to the severity of the disease or disorder, previous treatments, the general health and/or age of the subject, and other diseases present. It will also be appreciated that the effective dosage of the antagomir used for treatment may increase or decrease over the course of a particular treatment. Changes in dosage may result and become apparent from the results of diagnostic assays. For example, the subject can be monitored after administering a composition of the present disclosure. Based on information from the monitoring, an additional amount of the composition of the present disclosure can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50's found to be effective in in vitro and in vivo animal models.

In certain embodiments, a miRNA, or a mimic or antagomir thereof, can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol (e.g., about 4.4×1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol per kg of bodyweight. The unit dose, for example, can be administered by injection (e.g., intravenous or intramuscular, intrathecally, intratumorally or directly into an organ), inhalation, or a topical application.

In certain embodiments, delivery of a miRNA, or a mimic or antagomir thereof, directly to an organ can be at a dosage on the order of about 0.00001 mg to about 3 mg per organ, or particularly about 0.0001-0.001 mg per organ, about 0.03-3.0 mg per organ, about 0.1-3.0 mg per organ or about 0.3-3.0 mg per organ.

In one embodiment, the unit dose is administered once a day, e.g., or less frequently less than or at about every 2, 4, 8 or 30 days. In another embodiment, the unit dose is not administered with a frequency (e.g., not a regular frequency). For example, the unit dose may be administered a single time. Because compositions of the present disclosure can persist for several days after administering, in many instances, it is possible to administer the composition with a frequency of less than once per day, or, for some instances, only once for the entire therapeutic regimen.

A miRNA, or a mimic or antagomir thereof, of the disclosure can be administered in a single dose or in multiple doses. Where the administration is by infusion, the infusion can be a single sustained dose or can be delivered by multiple infusions. Injection can be directly into the tissue at or near the site of interest. Multiple injections of can be made into the tissue at or near the site.

In a particular dosage regimen, the miRNA, or a mimic or antagomir thereof, is injected at or near a disease site once a day for seven days, for example, into a tumor, a tumor bed, or tumor vasculature. Where a dosage regimen comprises multiple administrations, it is understood that the effective amount of the miRNA, or mimic or antagomir thereof, administered to the subject can include the total amount of miRNA, or mimic or antagomir thereof, administered over the entire dosage regimen. One skilled in the art will appreciate that the exact individual dosages may be adjusted somewhat depending on a variety of factors, including the specific antagomir being administered, the time of administration, the route of administration, the nature of the formulation, the rate of excretion, the particular disorder being treated, the severity of the disorder, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns can be determined by the attending physician in consideration of the above-identified factors.

In one embodiment, a subject is administered an initial dose, and one or more maintenance doses of a miRNA, or a mimic or antagomir thereof. The maintenance dose or doses are generally lower than the initial dose, e.g., one-half less of the initial dose. The maintenance doses are generally administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

### Detection Methods

### Nucleic Acid Detection Methods

In certain embodiments, it may prove useful to assess the expression of miRNAs (e.g., miR-34a-5p, miR-99b-5p, miR-96-5p) in a cell from a subject having or suspected of having cancer, including prostate cancer, breast cancer, lung cancer, and colon cancer as well as aggressive forms of cancer (e.g., castration-resistant prostate cancer). Any method of detection known to one of skill in the art falls within the general scope of the present disclosure. Various aspects of nucleic acid detection are discussed below.

Nucleic acids can used be as probes or primers for embodiments involving nucleic acid hybridization. The use of a probe or primer of between 13 and 100 nucleotides, preferably between 17 and 100 nucleotides in length, or in some aspects of the disclosure up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the disclosure may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In certain embodiments, hybridization may be achieved under conditions of, for example, 50 mM Tris-HCl (pH 8.3), 75 mM KCl, 3 mM MgCl2, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl2, at temperatures ranging from approximately 40° C. to about 72° C.

In certain embodiments, it will be advantageous to employ nucleic acids of defined sequences of the present disclosure in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present disclosure are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Nucleic acids used as a template for amplification may be isolated from cells, tissues or other samples according to standard methodologies (Sambrook and Russell, Molecular Cloning: A Laboratory Manual 3rd Ed., Cold Spring Harbor Laboratory Press, 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In certain embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, J. Macromol. Sci. Pure Appl. Chem., A31(1): 1355-1376, 1994).

A number of template dependent processes are available to amplify the oligonucleotide sequences present in a given template sample. One of the best known amplification methods is the polymerase chain reaction (PCR) which is described in detail in U.S. Pat. Nos. 4,683,195, 4,683,202 and 4,800,159, and in Innis et al. (Proc. Natl. Acad. Sci. USA, 85(24):9436-9440, 1988), each of which is incorporated herein by reference in their entirety.

A reverse transcriptase PCR amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific miRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, may also be used.

Alternative methods for amplification of target nucleic acid sequences that may be used in the practice of the present disclosure are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present disclosure. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., Proc. Natl. Acad. Sci. USA, 89:392-396, 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., Proc. Natl. Acad. Sci. USA, 86:1173, 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present disclosure.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, In: PCR Protocols: A Guide To Methods And Applications, Academic Press, N.Y., 1990; Ohara et al., Proc. Natl. Acad. Sci. USA, 86:5673-5677, 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and cluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present disclosure, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

The northern blot is a technique used in molecular biology research to study gene expression by detection of RNA in a sample. With northern blotting it is possible to observe cellular control over structure and function by determining the particular gene expression levels during differentiation, morphogenesis, as well as abnormal or diseased conditions. Northern blotting involves the use of electrophoresis to separate RNA samples by size and detection with a hybridization probe complementary to part of or the entire target sequence. The term ‘northern blot’ actually refers specifically to the capillary transfer of RNA from the electrophoresis gel to the blotting membrane. However, the entire process is commonly referred to as northern blotting.

A general blotting procedure starts with extraction of total RNA from a homogenized tissue sample or from cells. Eukaryotic mRNA can then be isolated through the use of oligo (dT) cellulose chromatography to isolate only those RNAs with a poly(A) tail. RNA samples are then separated by gel electrophoresis. Since the gels are fragile and the probes are unable to enter the matrix, the RNA samples, now separated by size, are transferred to a nylon membrane through a capillary or vacuum blotting system.

A nylon membrane with a positive charge is the most effective for use in northern blotting since the negatively charged nucleic acids have a high affinity for them. The transfer buffer used for the blotting usually contains formamide because it lowers the annealing temperature of the probe-RNA interaction, thus eliminating the need for high temperatures, which could cause RNA degradation. Once the RNA has been transferred to the membrane, it is immobilized through covalent linkage to the membrane by UV light or heat. After a probe has been labeled, it is hybridized to the RNA on the membrane. Experimental conditions that can affect the efficiency and specificity of hybridization include ionic strength, viscosity, duplex length, mismatched base pairs, and base composition. The membrane is washed to ensure that the probe has bound specifically and to prevent background signals from arising. The hybrid signals are then detected by X-ray film and can be quantified by densitometry. To create controls for comparison in a northern blot samples not displaying the gene product of interest can be used after determination by microarrays or RT-PCR.

The RNA samples are most commonly separated on agarose gels containing formaldehyde as a denaturing agent for the RNA to limit secondary structure. The gels can be stained with ethidium bromide (EtBr) and viewed under UV light to observe the quality and quantity of RNA before blotting. Polyacrylamide gel electrophoeresis with urea can also be used in RNA separation but it is most commonly used for fragmented RNA or microRNAs. An RNA ladder is often run alongside the samples on an electrophoresis gel to observe the size of fragments obtained but in total RNA samples the ribosomal subunits can act as size markers. Since the large ribosomal subunit is 28S (approximately 5 kb) and the small ribosomal subunit is 18S (approximately 2 kB) two prominent bands appear on the gel, the larger at close to twice the intensity of the smaller.

Probes for northern blotting are composed of nucleic acids with a complementary sequence to all or part of the RNA of interest, they can be DNA, RNA, or oligonucleotides with a minimum of 25 complementary bases to the target sequence. RNA probes (riboprobes) that are transcribed in vitro are able to withstand more rigorous washing steps preventing some of the background noise. Commonly cDNA is created with labelled primers for the RNA sequence of interest to act as the probe in the northern blot. The probes must be labelled either with radioactive isotopes (32P) or with chemiluminescence in which alkaline phosphatase or horseradish peroxidase break down chemiluminescent substrates producing a detectable emission of light. The chemiluminescent labelling can occur in two ways: either the probe is attached to the enzyme, or the probe is labelled with a ligand (e.g., biotin) for which the antibody (e.g., avidin or streptavidin) is attached to the enzyme. X-ray film can detect both the radioactive and chemiluminescent signals and many researchers prefer the chemiluminescent signals because they are faster, more sensitive, and reduce the health hazards that go along with radioactive labels. The same membrane can be probed up to five times without a significant loss of the target RNA.

Chip-based DNA technologies such as those described by Hacia et al. (Nature Genet., 14:441-449, 1996) and Shoemaker et al. (Nature Genetics, 14:450-456, 1996) are contemplated. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., Proc. Natl. Acad. Sci. USA, 91:5022-5026, 1994; and Fodor et al., Science, 251:767-773, 1991). It is contemplated that this technology may be used in conjunction with evaluating the expression level of a miRNA with respect to diagnostic, as well as preventative and treatment methods of the disclosure.

The present disclosure may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present disclosure and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532, 128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### Protein Detection Methods

In certain embodiments, it may prove useful to assess the level of proteins (HIF1A, IGFBP2, PIK3CB, MTOR, or MAPKAPK2) in a cell from a subject having or suspected of having cancer, including prostate cancer, breast cancer, lung cancer, and colon cancer as well as aggressive forms of cancer (e.g., castration-resistant prostate cancer). Any method of detection known to one of skill in the art falls within the general scope of the present disclosure. Methods to measure protein level include ELISA (enzyme linked immunosorbent assay), western blot, immunohistochemistry (IHC), and immunofluorescence using detection reagents such as an antibody or protein binding agents. Various aspects of protein detection are discussed below.

The protein can be detected by antibodies against the protein in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein can be used. In certain embodiments, the tissue sample is contacted with a primary antibody for a specific target for a period of time sufficient for the antibody-target binding to occur. The antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In certain embodiments, an IHC assay is performed with an unlabeled primary antibody in conjunction with a labeled secondary antibody in an indirect assay. The indirect assay utilizes two antibodies for the detection of the protein in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3,3′-diaminobenzidine (DAB) to produce brown precipitate at the reaction site.

In certain embodiments, the expression level of the protein can also be detected with antibodies against the protein using an immunoblotting assay. In certain embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein can be used. In certain embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In certain embodiments, unlabeled primary antibody is used in an indirect assay in conjunction with a labeled secondary antibody specific for the primary antibody. The secondary antibodies can be labeled, for example, with enzymes or other detectable labels such as fluorescent labels, luminescent labels, colorimetric labels, or radioisotopes. Immunoblotting protocols and kits are well known in the art and are commercially available. Immunoblotting includes, but is not limited to, western blot, in-cell western blot, and dot blot. Dot blot is a simplified procedure in which protein samples are not separated by electrophoresis but are spotted directly onto a membrane. In cell western blot involves seeding cells in microtiter plates, fixing/permeabilizing the cells, and subsequent detection with a primary labeled primary antibody or unlabeled primary antibody followed by labeled secondary antibody.

In certain embodiments, the levels of the protein can also be detected with the antibodies in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein can be used. In certain embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In certain embodiments, unlabeled primary antibody is used in an indirect assay in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the level of the target protein. Non-fluorescent cytoplasmic proteins can also be observed by staining permeabilized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In certain embodiments, the levels of the protein products can also be detected using immunoassays such as an Enzyme Immune Assay (EIA) or an ELISA. Both EIA and ELISA assays are known in the art, e.g. for assaying a wide variety of tissues and samples, including blood, plasma, serum or tumor tissue. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays arc commonly used assay format. A number of variations of the sandwich assay technique exist. For example, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In certain embodiments of the EIA or ELISA assays, an enzyme is conjugated to the second antibody. In certain embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art.

For the immunoassays, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present disclosure.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available. Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare.

In certain embodiments of the immunoassays, a detectable label can be directly coupled to cither the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5- (or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences), and the like. Exemplary chromophores include, but are not limited to, phenolphthalein, malachite green, nitroaromatics such as nitrophenyl, diazo dyes, dabsyl (4-dimethylaminoazobenzenc-4′-sulfonyl), and the like.

### Vectors for Cloning, Gene Transfer, and Expression

In certain embodiments, expression vectors are employed to express nucleic acid agonist or antagonists, such as miRNAs, antisense molecules. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. Generally, the nucleic acid encoding a gene product is under transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (Sambrook et al., In: Molecular Cloning: A Laboratory Manual, 2^{nd} Ed., Cold Spring Harbor Laboratory Press, Cold Spring Harbor, N.Y., 1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. The identity of tissue-specific promoters or clements, as well as assays to characterize their activity, is well known to those of skill in the art.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in certain embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

In certain embodiments, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

There are a number of ways in which expression vectors may be introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Hermonat and Muzycska, Proc. Natl. Acad. Sci. USA, 81:6466-6470, 1984; Baichwal and Sugden, In: Gene Transfer, Kucherlapati (Ed.), Plenum Press, NY, 117-148, 1986; Temin, In: Gene Transfer, Kucherlapati (Ed.), NY, Plenum Press, 149-188, 1986; Ridgeway, In: Vectors: A Survey of Molecular Cloning Vectors and Their Uses, Rodriguez et al. (Eds.), Stoneham: Butterworth, 467-492, 1988; Coupar et al., Gene, 68:1-10, 1988; Friedmann, Science, 244:1275-1281, 1989; Horwich et al. J. Virol., 64:642-650, 1990). The first viruses used as gene vectors were DNA viruses including the papovaviruses (simian virus 40, bovine papilloma virus, and polyoma) and adenoviruses. Vectors derived from viruses such as vaccinia virus, adeno-associated virus (AAV), and herpesviruses may be employed. They offer several attractive features for various mammalian cells. Defective hepatitis B viruses also are useful as expression vectors.

Several non-viral methods for the transfer of expression constructs into cultured mammalian cells also are contemplated by the present disclosure. These include calcium phosphate precipitation (Graham and Van Der Eb, Virology, 52:456-467, 1973; Chen and Okayama, Mol. Cell Biol., 7(8):2745-2752, 1987; Rippe, et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Natl. Acad. Sci. USA, 81:7161-7165, 1984), direct microinjection (Harland and Weintraub, J. Cell Biol., 101(3): 1094-1099, 1985), DNA-loaded liposomes (Nicolau and Sene, Biochim. Biophys. Acta, 721:185-190, 1982; Fraley et al., Proc. Natl. Acad. Sci. USA, 76:3348-3352, 1979) and lipofectamine-DNA complexes, cell sonication (Fechheimer, et al., Proc Natl. Acad. Sci. USA, 84:8463-8467, 1987), gene bombardment using high velocity microprojectiles (Yang et al., Proc. Natl. Acad. Sci. USA, 87:4144-4148, 1990), and receptor-mediated transfection (Wu and Wu, J. Biol. Chem., 262:4429-4432, 1987; Wu and Wu, Biochemistry, 27: 887-892, 1988). Some of these techniques may be successfully adapted for in vivo or ex vivo use.

Once the expression construct has been delivered into the cell the nucleic acid encoding the nucleic acid of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA, 81:7529-7533, 1984) successfully injected polyomavirus DNA in the form of calcium phosphate precipitates into liver and spleen of adult and newborn mice demonstrating active viral replication and acute infection. Benvenisty and Neshif (Proc. Natl. Acad. Sci. USA, 83(24):9551-9555, 1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In certain embodiments for transferring a naked DNA expression construct into cells may involve particle bombardment. This method depends on the ability to accelerate DNA-coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force. The microprojectiles used have consisted of biologically inert substances such as tungsten or gold beads.

Selected organs including the eye, liver, skin, and muscle tissue of rats and mice have been bombarded in vivo (Yang et al., Proc. Natl. Acad. Sci. USA, 87:4144-4148, 1990; Zelenin et al., FEBS Lett, 287(1-2): 118-120, 1991). This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In certain embodiments, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver Diseases, Targeted Diagnosis and Therapy Using Specific Receptors and Ligands, Wu et al. (Eds.), Marcel Dekker, NY, 87-104, 1991). Also contemplated are lipofectamine-DNA complexes.

Liposome-mediated nucleic acid delivery and expression of foreign DNA in vitro has been very successful. Wong et al. (Gene, 10:87-94, 1980) demonstrated the feasibility of liposome-mediated delivery and expression of foreign DNA in cultured chick embryo, HeLa and hepatoma cells. Nicolau et al., (Methods Enzymol., 149:157-176, 1987) accomplished successful liposome-mediated gene transfer in rats after intravenous injection.

In certain embodiments, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In certain embodiments, the liposome may be complexed or employed in conjunction with nuclear non-histone chromosomal proteins (HMG-1) (Kato et al, J. Biol. Chem., 266:3361-3364, 1991). In certain embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, J. Biol. Chem., 262:4429-4432, 1987) and transferrin (Wagner et al., Proc. Natl. Acad. Sci. USA 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci. USA, 91:4086-4090, 1994) and epidermal growth factor (EGF) has also been used to deliver genes to squamous carcinoma cells (EPO 0273085).

In certain embodiments, the delivery vehicle may comprise a ligand and a liposome. For example, Nicolau et al. (Methods Enzymol., 149:157-176, 1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, the oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from a subject, the delivery of a nucleic acid into the cells in vitro, and then the return of the modified cells back into a subject. This may involve the surgical removal of tissue/organs from a subject or the primary culture of cells and tissues.

### Kits

Any of the compositions described herein may be comprised in a kit. The kits may be designed for either therapeutic or diagnostic purposes. In a non-limiting example, an individual miRNA agonist or antagonists (e.g., expression construct, mimic, antagomir, LNA) is included in a kit. The kit may also include one or more transfection reagent(s) to facilitate delivery of the agonist or antagonist to cells. Alternatively, the kit may contain reagents designed to measure miRNA levels, such as probes and primers, as well as enzymes for performing diagnostic reactions (polymerases, detectable enzymes and labels, etc.).

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present disclosure also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present disclosure will also typically include a means for containing the vials in close confinement for commercial sale, such as, e.g., injection and/or blow-molded plastic containers into which the desired vials are retained.

Such kits may also include components that preserve or maintain the miRNA or that protect against its degradation. Such components may be RNase-free or protect against RNases. Such kits generally will comprise, in suitable means, distinct containers for each individual reagent or solution. A kit will also include instructions for employing the kit components as well the use of any other reagent not included in the kit. Instructions may include variations that can be implemented.

It is contemplated that such reagents are embodiments of kits of the disclosure. Such kits, however, are not limited to the particular items identified above and may include any reagent used for the manipulation or characterization of miRNA.

### Embodiments

The following numbered embodiments also form part of the present disclosure:

1. A method of treating cancer in a subject in need thereof, the method comprising administering to the subject a therapeutically effective amount of an agonist of miR-34a-5p, an agonist of miR-99b-5p, or an antagonist of miR-96-5p.

2. The method of embodiment 1, wherein the agonist of miR-34a-5p comprises a miR-34a-5p mimic.

3. The method of embodiment 1 or embodiment 2, wherein the miR-34a-5p mimic comprises a nucleotide sequence having at least about 80% identity to SEQ ID NO: 1.

4. The method of any one of embodiments 1-3, wherein the agonist of miR-34a-5p decreases expression of HIF1A, IGFBP2, and PIK3CB in the subject.

5. The method of any one of embodiments 1-4, wherein the agonist of miR-99b-5p comprises a miR-99b-5p mimic.

6. The method of any one of embodiments 1-5, wherein the miR-99b-5p mimic comprises having at least about 80% identity to SEQ ID NO: 2.

7. The method of any one of embodiments 1-6, wherein the agonist of miR-99b-5p decreases expression of MTOR in the subject.

8. The method of any one of embodiments 1-7, wherein the antagonist of miR-96-5p comprises a miR-96-5p antagomir.

9. The method of any one of embodiments 1-8, wherein the miR-96-5p antagomir comprises a nucleotide sequence having at least about 80% complementary to SEQ ID NO: 3.

10. The method of any one of embodiments 1-9, wherein the antagonist of miR-96-5p increases expression of MAPKAPK2 in the subject.

11. The method of any one of embodiments 1-10, wherein the subject has prostate cancer, breast cancer, lung cancer, or colon cancer.

12. The method of any one of embodiments 1-11, wherein the subject has an aggressive form of cancer.

13. The method of any one of embodiments 1-12, wherein the subject has castration-resistant prostate cancer.

14. The method of any one of embodiments 1-13, wherein the subject is a human.

15. The method of any one of embodiments 1-14, wherein the subject is of African ancestry.

16. The method of any one of embodiments 1-15, further comprising administering to the subject an anti-cancer therapy.

17. The method of any one of embodiments 1-16, wherein the anti-cancer therapy comprises chemotherapy, radiotherapy, immunotherapy, surgical resection, or gene therapy.

18. The method of any one of embodiments 1-17, wherein the chemotherapy comprises docetaxel.

19. A method of identifying a subject having or at risk of developing cancer, the method comprising assessing the level of miR-34a-5p, miR-99b-5p, or miR-96-5p in a sample from the subject.

20. The method of embodiment 19, wherein decreased miR-34a-5p, decreased miR-99b-5p, or increased miR-96-5p as compared to a control is indicative that the subject has or is at risk of developing prostate cancer, breast cancer, lung cancer, or colon cancer.

21. The method of embodiment 19 or embodiment 20, wherein decreased miR-34a-5p, decreased miR-99b-5p, or increased miR-96-5p as compared to a control is indicative that the subject has or is at risk of developing an aggressive form of cancer.

22. The method of any one of embodiments 19-21, wherein each of miR-34a-5p, miR-99b-5p, and miR-96-5p are assessed.

23. The method of any one of embodiments 19-22, further comprising assessing the level of HIF1A, IGFBP2, PIK3CB, MTOR, or MAPKAPK2 in the sample.

24. The method of any one of embodiments 19-23, wherein the subject is a human.

25. The method of any one of embodiments 19-24, wherein the subject is of African ancestry.

26. The method of any one of embodiments 19-25, further comprising administering a therapeutically effective amount of an agonist of miR-34a-5p, an agonist of miR-99b-5p, or an antagonist of miR-96-5p to a subject identified as having or at risk of developing cancer.

27. The method of any one of embodiments 19-26, wherein the agonist of miR-34a-5p comprises a miR-34a-5p mimic.

28. The method of any one of embodiments 19-27, wherein the miR-34a-5p mimic comprises a nucleotide sequence having at least about 80% identity to SEQ ID NO: 1.

29. The method of any one of embodiments 19-28, the agonist of miR-34a-5p decreases expression of HIF1A, IGFBP2, and PIK3CB in the subject.

30. The method of any one of embodiments 19-29, wherein the agonist of miR-99b-5p comprises a miR-99b-5p mimic.

31. The method of any one of embodiments 19-30, wherein the miR-99b-5p mimic comprises having at least about 80% identity to SEQ ID NO: 2.

32. The method of any one of embodiments 19-31, wherein the agonist of miR-99b-5p decreases expression of MTOR in the subject.

33. The method of any one of embodiments 19-32, wherein the antagonist of miR-96-5p comprises a miR-96-5p antagomir.

34. The method of any one of embodiments 19-33, wherein the miR-96-5p antagomir comprises a nucleotide sequence having at least about 80% complementary to SEQ ID NO: 3.

35. The method of any one of embodiments 19-34, wherein the antagonist of miR-96-5p increases expression of MAPKAPK2 in the subject.

36. The method of any one of embodiments 19-35, further comprising administering an anti-cancer therapy to a subject identified as having or at risk of developing cancer.

37. The method of any one of embodiments 19-36, wherein the anti-cancer therapy comprises chemotherapy, radiotherapy, immunotherapy, surgical resection, or gene therapy.

38. The method of any one of embodiments 19-37, wherein the chemotherapy comprises docetaxel.

39. A pharmaceutical composition comprising a miR-34a-5p mimic, a miR-99b-5p mimic, or a miR-96-5p antagomir; and a pharmaceutically acceptable carrier.

40. The pharmaceutical composition of embodiment 39, wherein the miR-34a-5p mimic comprises a nucleotide sequence having at least about 80% identity to SEQ ID NO: 1

41. The pharmaceutical composition of embodiment 39 or embodiment 40, wherein the miR-99b-5p mimic comprises a nucleotide sequence having at least about 80% identity to SEQ ID NO: 2,

42 The pharmaceutical composition of any one of embodiments 39-41, wherein the miR-96-5p antagomir comprises a nucleotide sequence having at least about 80% complementary to SEQ ID NO: 3.

43. The pharmaceutical composition of any one of embodiments 39-42, wherein the composition comprises the miR-34a-5p mimic, the miR-99b-5p mimic, and the miR-96-5p antagomir.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: MicroRNA-mRNA Regulatory Network Mediates Activation of mTOR and VEGF Signaling in African American Prostate Cancer

MiRNAs play important roles in either promoting or preventing cancer development and/or progression. MiRNAs promoting tumorigenesis are referred as oncogenic miRNAs or ‘oncomirs’, while miRNAs function as tumor suppressor are termed tumor suppressive miRNAs. Accumulating evidence indicates that miRNAs and their regulatory and biogenesis mechanisms are involved in the development of prostate cancer. Previous studies have shown that let-7a, let-7c, miR-15a, miR-20, miR-24, miR-29, miR-125b, miR-128a, miR-143, miR-145, miR-181a, miR-181b, and miR-222 are downregulated in both prostate cancer cell lines and/or tissues, whereas let-7d*, miR-17-5p, miR-21, miR-141, miR-148a, miR-182, miR-200b, miR-200c, and miR-375 are upregulated in prostate cancer cells and/or tissues. Recent studies further shed light on deciphering the miRNA-mediated mechanisms underlying prostate cancer disparities. For instance, overexpression of miR-130b has been linked to prostate cancer aggressiveness and poor clinical outcome in African Americans. A previous study has also shown that miR-182 is upregulated in African American prostate cancer vs. European American prostate cancer, and the high-level miR-182 is correlated with poorer survival rate in African American prostate cancer. In addition, downregulation of miR-34b and miR-146a have been observed in African American prostate cancer vs. European American prostate cancer. AR and ETV1 were shown to be directly targeted by miR-34b, and the downregulation of miR-34b enhances AR expression and promotes cell proliferation in African American prostate cancer. Moreover, a systems biology approach was previously employed to evaluate the functional impacts of population-associated miRNAs and mRNAs in a three-way comparison in prostate cancer (African American prostate cancer vs. European American prostate cancer, African American prostate cancer vs. African American normal, European American prostate cancer vs. European American normal). ErbB signaling has been identified as a critical cascade significantly upregulated by miRNA-mRNA regulatory network in African American prostate cancer.

In this example, the genomic data was revisited and the 10 differently expressed miRNAs identified between African American and European American prostate cancer were particularly focused on, to further assess their functional impacts in African American prostate cancer disparities. Specifically, an integrative genomic approach was performed, combining a miRNA-driven pathway analysis algorithm with miRNA target prediction and unique mRNA mapping, to identify cancer signaling pathways significantly influenced by the miRNA-mRNA regulatory network in African American prostate cancer. Through this analysis, 58 significant pathways regulated by miRNAs and their target mRNAs in African American prostate cancer were identified. Notably, ErbB-PI3K-AKT-mTOR-HIF-VEGF axis was identified as central signaling cascade highly regulated by African American-depleted/enriched miRNAs and mRNAs in African American prostate cancer. A novel panel of reciprocal miRNA-mRNA pairings were defined as core miRNA-mRNA regulatory components within mTOR and VEGF signaling, potentially serving as potential precision biomarkers and novel therapeutic targets for African American prostate cancer.

### Results

MirPath v.3 Analysis Reveals 68 Signaling Pathways Differentially Regulated by miRNA-mRNA Networks in African American and European American Prostate Cancer

In previous genomic studies, total RNA samples isolated from 20 African American and 15 European American prostate cancer specimens were subjected to miRNA profiling and mRNA profiling analysis. 10 miRNAs were found differentially expressed between African American and European American prostate cancer, including 8 African American-depleted miRNAs (downregulated in African American prostate cancer vs. European American prostate cancer) and 2 African American-enriched miRNAs (upregulated in African American prostate cancer vs. European American prostate cancer) (TABLE 1). By importing these 10 African American-depleted/enriched miRNAs into the mirPath v.3 algorithm, 58 KEGG pathways were identified as significant pathways (adjusted p-value<0.05) differentially regulated between African American prostate cancer and European American prostate cancer (TABLE 2). Several canonical oncogenic signaling pathways (such as ErbB, mTOR, HIF-1, VEGF and focal adhesion pathways) were significantly influenced by the African American-depleted/enriched miRNAs (TABLE 2). In addition, critical signaling pathways related to cancer, cell cycle, inflammation and cell death regulations (such as p53, cell cycle, insulin, NF-kappa B and TNF signaling) were also differentially regulated in African American prostate cancer vs. European American prostate cancer (TABLE 2).

TABLE 1
10 differentially expressed miRNAs between African American prostate cancer and European
American prostate cancer. The African American-depleted miRNAs are defined as the miRNAs
significantly downregulated in African American prostate cancer vs. European American
prostate cancer, while African American-enriched miRNAs are defined as miRNAs significantly
upregulated in African American prostate cancer vs. European American prostate cancer.
Significance was determined based on ANOVA with FDR < 0.1 (adjust p-value) after
multiple correction from miRNA microarray data. The previous miRNA IDs and current
miRNA IDs (most updated IDs from miRbase) are included in this Table. The current miRNA
IDs were imported to DIANA-mirPath V.3 for further pathway analysis.

| | | Fold change | Regulation | | |
|---|---|---|---|---|---|
| | | (African | (African | | |
| | | American | American | | |
| | | prostate | prostate | | |
| | | cancer vs. | cancer vs. | African | |
| | | European | European | American (AA)- | |
| | | American | American | enriched or AA- | Adjust p- |
| miRNA | miRNA | prostate | prostate | depleted | value, <10% |
| (previous ID) | (current ID) | cancer) | cancer) | miRNA | FDR |
| hsa-miR-125b-2* | hsa-miR-125b-2-3p | −1.3902426 | downregulated | AA-depleted | yes |
| hsa-miR-758 | hsa-miR-758-3p | −1.686261 | downregulated | AA-depleted | yes |
| hsa-miR-99b | hsa-miR-99b-5p | −1.4173684 | downregulated | AA-depleted | yes |
| hsa-miR-133a | hsa-miR-133a-5p | −1.4290309 | downregulated | AA-depleted | yes |
| hsa-miR-34a | hsa-miR-34a-5p | −1.4193252 | downregulated | AA-depleted | yes |
| hsa-miR-96 | hsa-miR-96-5p | 1.7657517 | upregulated | AA-enriched | yes |
| hsa-miR-130b | hsa-miR-130b-3p | 1.4793279 | upregulated | AA-enriched | yes |
| hsa-miR-542-5p | hsa-miR-542-5p | −1.7648832 | downregulated | AA-depleted | yes |
| hsa-miR-572 | hsa-miR-572 | −1.7938243 | downregulated | AA-depleted | yes |
| hsa-miR-378* | hsa-miR-378a-5p | −1.4021478 | downregulated | AA-depleted | yes |

TABLE 2
KEGG pathways differentially regulated by population-associated
miRNAs between African American and European American prostate
cancer. MirPath V.3 program was used for identification
of the significant signaling pathways regulated by African
American-depleted and enriched miRNAs. The significance
was measured based on adjusted p-value (FDR < 0.05)
determined by statistics implemented in mirPath V.3.

| KEGG Pathway | p-Value | #Genes | #miRNAs |
|---|---|---|---|
| MicroRNAs in cancer | 3.88 × 10⁻⁵⁴ | 100 | 8 |
| Proteoglycans in cancer | 8.70 × 10⁻¹² | 99 | 8 |
| Adherens junction | 4.02 × 10⁻¹⁰ | 45 | 7 |
| Viral carcinogenesis | 1.29 × 10⁻⁷ | 105 | 8 |
| Glioma | 3.22 × 10⁻⁷ | 39 | 8 |
| Transcriptional misregulation in | 3.22 × 10⁻⁷ | 88 | 8 |
| cancer | | | |
| Cell cycle | 4.05 × 10⁻⁷ | 71 | 7 |
| Prostate cancer | 6.70 × 10⁻⁷ | 55 | 8 |
| Pancreatic cancer | 1.27 × 10⁻⁶ | 44 | 7 |
| Bacterial invasion of epithelial | 2.81 × 10⁻⁶ | 44 | 8 |
| cells | | | |
| Colorectal cancer | 4.14 × 10⁻⁶ | 40 | 7 |
| Thyroid cancer | 9.18 × 10⁻⁶ | 21 | 6 |
| Fatty acid biosynthesis | 1.35 × 10⁻⁵ | 5 | 3 |
| ErbB signaling pathway | 1.35 × 10⁻⁵ | 48 | 8 |
| Pathways in cancer | 1.89 × 10⁻⁵ | 166 | 8 |
| Chronic myeloid leukemia | 2.45 × 10⁻⁵ | 43 | 6 |
| Central carbon metabolism in | 3.86 × 10⁻⁵ | 39 | 7 |
| cancer | | | |
| Non-small cell lung cancer | 5.33 × 10⁻⁵ | 34 | 6 |
| Shigellosis | 8.00 × 10⁻⁵ | 37 | 7 |
| Regulation of actin cytoskeleton | 9.30 × 10⁻⁵ | 100 | 7 |
| Renal cell carcinoma | 1.14 × 10⁻⁴ | 39 | 6 |
| p53 signaling pathway | 1.14 × 10⁻⁴ | 42 | 7 |
| Glycosphingolipid biosynthesis- | 1.16 × 10⁻⁴ | 13 | 5 |
| lacto and neolacto series | | | |
| Endometrial cancer | 1.85 × 10⁻⁴ | 31 | 6 |
| Bladder cancer | 2.18 × 10⁻⁴ | 26 | 8 |
| Hepatitis B | 2.30 × 10⁻⁴ | 68 | 7 |
| Other types of O-glycan | 3.88 × 10⁻⁴ | 15 | 5 |
| biosynthesis | | | |
| Alcoholism | 4.33 × 10⁻⁴ | 88 | 8 |
| Endocytosis | 5.08 × 10⁻⁴ | 93 | 7 |
| Acute myeloid leukemia | 6.12 × 10⁻⁴ | 33 | 6 |
| Melanoma | 8.26 × 10⁻⁴ | 38 | 8 |
| Neurotrophin signaling pathway | 1.746 × 10⁻³ | 58 | 8 |
| Focal adhesion | 2.185 × 10⁻³ | 96 | 8 |
| mTOR signaling pathway | 2.477 × 10⁻³ | 33 | 6 |
| HIF-1 signaling pathway | 2.827 × 10⁻³ | 53 | 8 |
| Estrogen signaling pathway | 2.827 × 10⁻³ | 44 | 8 |
| Protein processing in | 2.962 × 10⁻³ | 75 | 7 |
| endoplasmic reticulum | | | |
| Oocyte meiosis | 2.974 × 10⁻³ | 55 | 7 |
| N-Glycan biosynthesis | 3.345 × 10⁻³ | 26 | 7 |
| Fc gamma R-mediated | 5.007 × 10⁻³ | 46 | 6 |
| phagocytosis | | | |
| Hippo signaling pathway | 5.356 × 10⁻³ | 64 | 7 |
| RNA transport | 5.788 × 10⁻³ | 76 | 6 |
| Thyroid hormone signaling | 6.076 × 10⁻3 | 58 | 7 |
| pathway | | | |
| Progesterone-mediated oocyte | 1.0333 × 10⁻² | 44 | 6 |
| maturation | | | |
| Salmonella infection | 1.1305 × 10⁻² | 42 | 6 |
| Axon guidance | 1.2697 × 10⁻² | 54 | 6 |
| Ubiquitin mediated proteolysis | 1.2697 × 10⁻² | 62 | 7 |
| Oxytocin signaling pathway | 1.2697 × 10⁻² | 72 | 8 |
| Insulin signaling pathway | 1.5334 × 10⁻² | 65 | 8 |
| VEGF signaling pathway | 1.5546 × 10⁻² | 32 | 5 |
| Glycosaminoglycan | 1.9381 × 10⁻² | 32 | 5 |
| biosynthesis-keratan sulfate | | | |
| Prolactin signaling pathway | 2.3342 × 10⁻² | 35 | 6 |
| Small cell lung cancer | 2.7242 × 10⁻² | 41 | 7 |
| FoxO signaling pathway | 3.0606 × 10⁻² | 62 | 8 |
| NF-kappa B signaling pathway | 3.4130 × 10⁻² | 38 | 7 |
| TNF signaling pathway | 3.6058 × 10⁻² | 47 | 6 |
| HTLV-I infection | 4.0449 × 10⁻² | 107 | 8 |
| Choline metabolism in cancer | 4.1519 × 10⁻² | 46 | 7 |

The mTOR and VEGF Signaling Pathways are Upregulated in African American Prostate Cancer Compared to European American Prostate Cancer

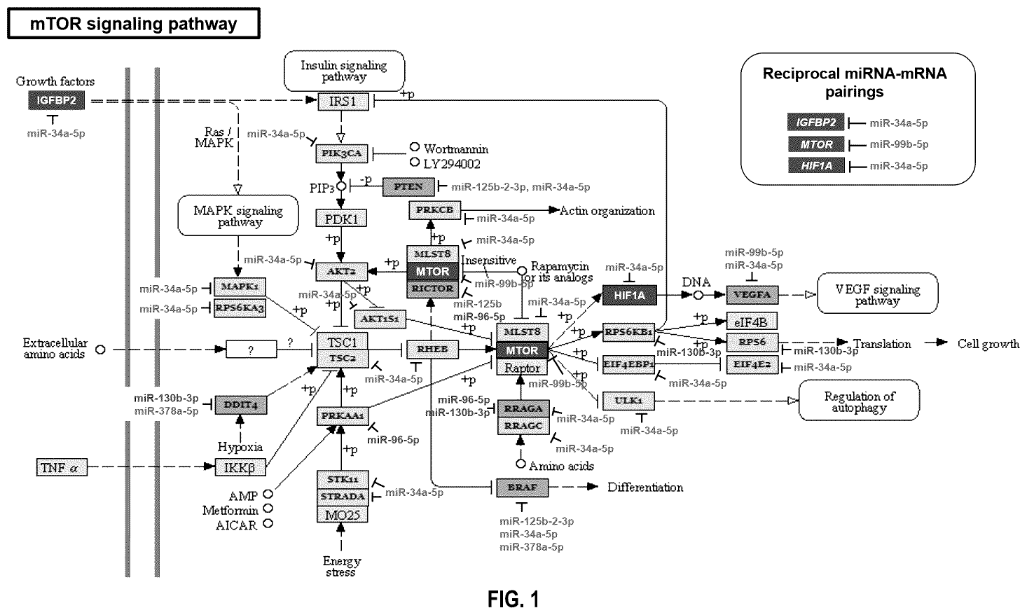

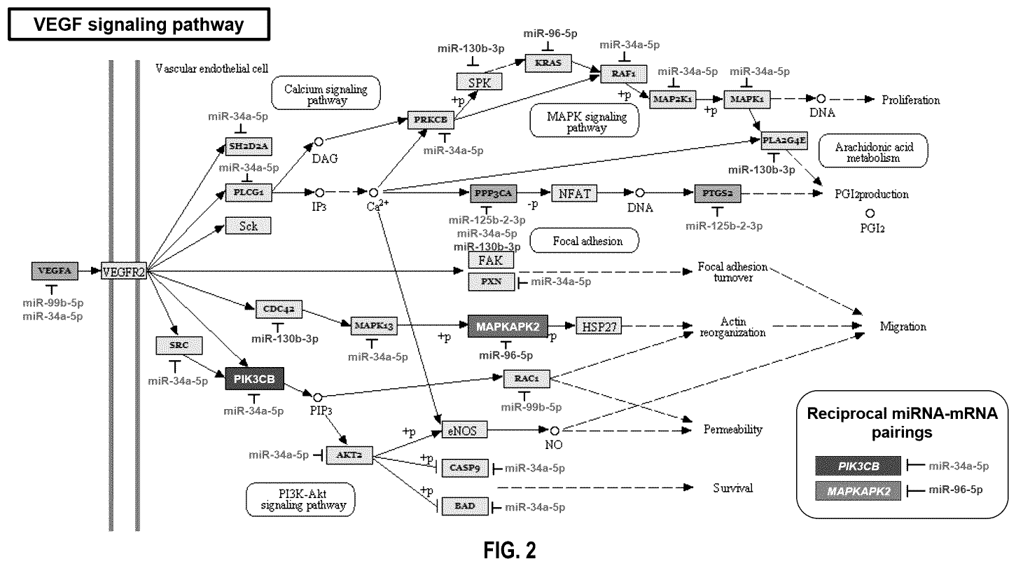

Previously, the ErbB signaling pathway was identified, by employing global test using mRNA profiling data and coupling with miRNA mapping, as a significant pathway that is highly activated by miRNA-mRNA interaction in African American prostate cancer. In this study, the miRNA-driven pathway analysis using mirPath v3 was first performed, followed by mapping of differentially expressed mRNAs in the identified pathways. Consistent with the previous finding, ErbB signaling ranks as a top canonical pathway significantly regulated by African American depleted/enriched miRNAs and mRNAs (adjusted p-value=1.35×10⁻⁵, TABLE 2). Notably, downstream of ErbB (or EGFR) signaling, the mTOR, HIF1A and VEGF signaling were also identified as significant signaling pathways influenced by African American depleted/enriched miRNAs and mRNAs (TABLE 2). These results suggest that ErbB (EGFR)-mTOR-HIF1A-VEGF axis as a critical signaling cascade mediated by miRNA-mRNA regulatory network in African American prostate cancer. To understand the miRNA-mRNA regulation in this signaling cascade, a detailed miRNA/mRNA mapping in mTOR (HIF1A is part of the KEGG mTOR signaling) and VEGF signaling in African American prostate cancer vs. European American prostate cancer was conducted. In KEGG mTOR signaling (FIG. 1), three oncogenes (IGFBP2, MTOR and HIF1A) were upregulated in African American prostate cancer vs. European American prostate cancer. The majority of KEGG mTOR signaling components (25 out of 38 genes) were predicted to be targeted by African American-depleted miRNAs (miR-34a-5p, miR-99b-5p, miR-125b-2-3p and miR-378a-5p, which were downregulated in African American prostate cancer vs. European American prostate cancer), and only five genes were predicted to be targeted by African American-enriched miRNAs (miR-96-5p and miR-130b-3p, which were upregulated in African American prostate cancer vs. European American prostate cancer). From the observed miRNA/mRNA mapping results, the mTOR signaling is theoretically upregulated in African American prostate cancer compared to European American prostate cancer. Similarly, 60% of VEGF signaling components (17 out of 28 genes) were predicted to be targeted by African American-depleted miRNAs (miR-34a-5p and miR-125b-2-3p), and six genes targeted by African American-enriched miRNAs (miR-96-5p and miR-130b-3p) (FIG. 2). These results implicate that VEGF signaling is preferably upregulated in African American prostate cancer when compared to European American prostate cancer. Reciprocal miRNA-mRNA pairings defined from miRNA and mRNA profiling data (down/up or up/down in miRNA/mRNA expression in African American prostate cancer vs. European American prostate cancer) represent the most robust miRNA-mRNA interaction that can be experimentally validated. In the mTOR and VEGF signaling pathways, five miRNA-mRNA reciprocal pairings in African American prostate cancer were identified. These miRNA-mRNA pairings included: miR-34a-5p/IGFBP2 (down/up), miR-34a-5p/HIF1A (down/up), miR-34a-5p/PIK3CB (down/up), miR-99b-5p/MTOR (down/up), and miR-96-5p/MAPKAPK2 (up/down) (FIG. 1, FIG. 2).

RT-qPCR Validations and Western Blot Analysis for the Candidate Reciprocal miRNA-mRNA Pairings in European American and African American Prostate Cancer Tissues and Cell Models

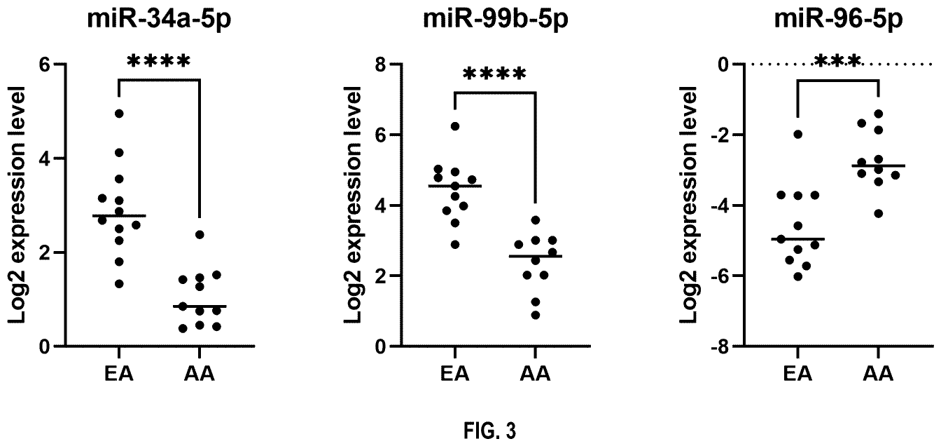

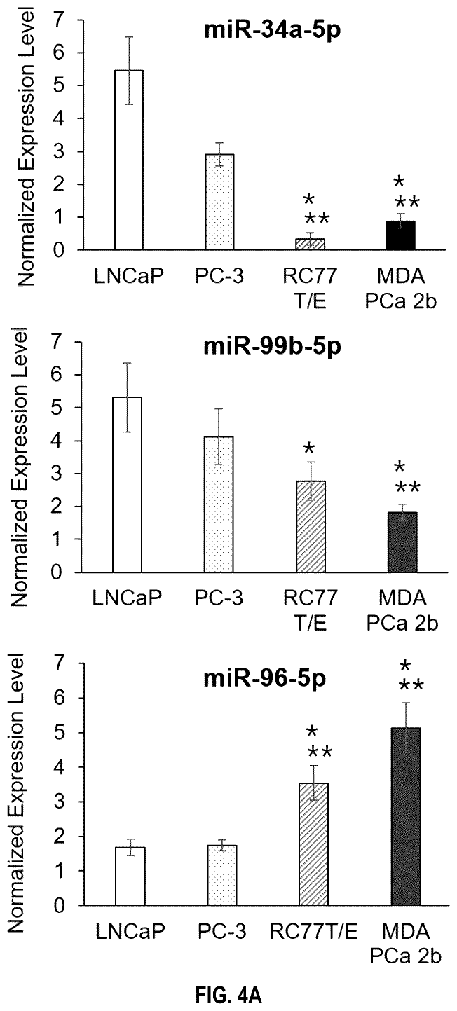

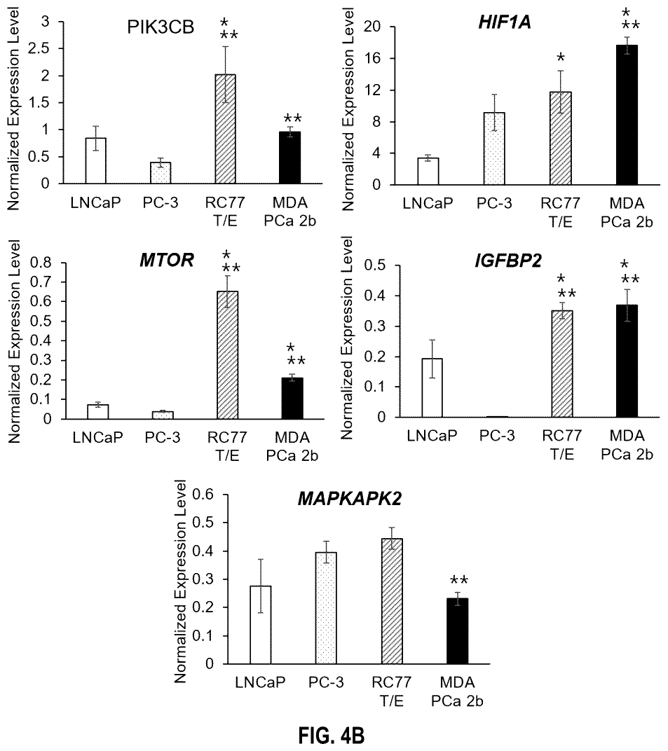

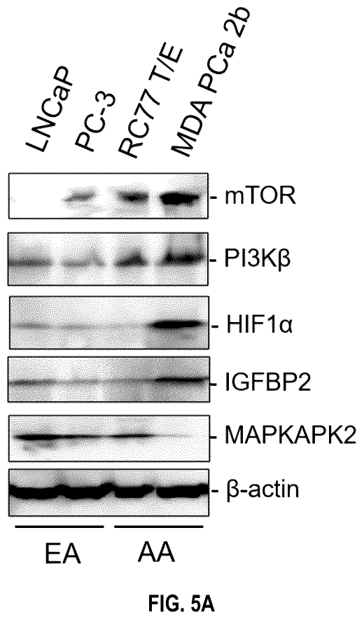

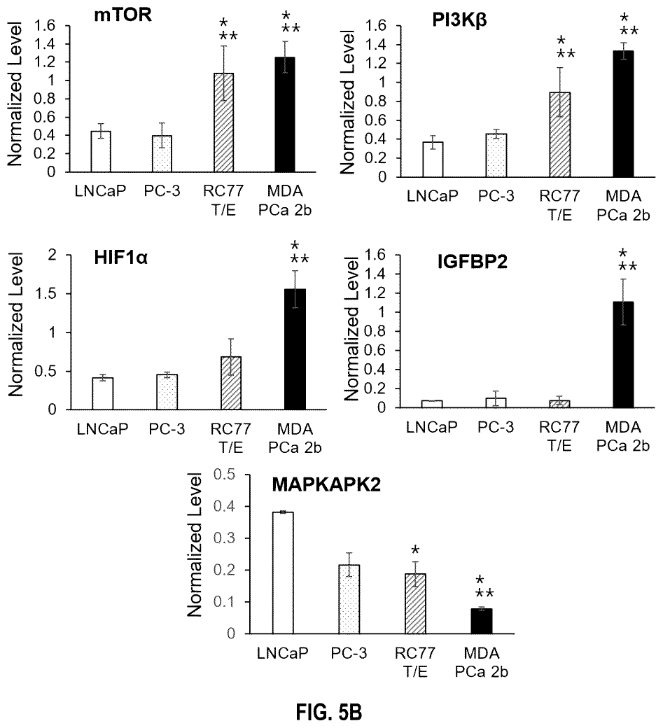

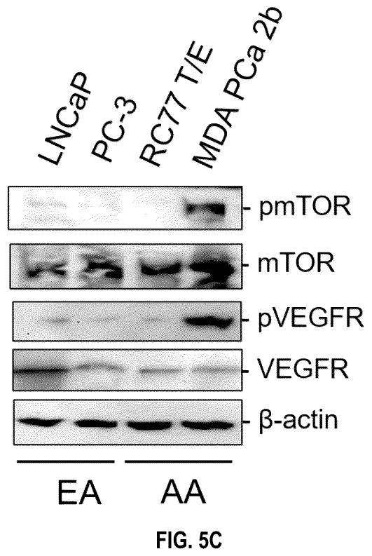

To verify the expression level of the candidate miRNA-mRNA pairings, RT-qPCR assays were performed to examine the miRNA and mRNA expression levels in European American prostate cancer and African American prostate cancer patient specimens and cell line models. RNA samples from 11 European American prostate cancer and 10 African American prostate cancer needle biopsy specimens were subjected to RT-qPCR validation of miR-34a-5p, miR-99b-5p, and miR-96-5p expression levels. The RT-qPCR validation confirmed that miR-34a-5p and miR-99b-5p were downregulated, while miR-96-5p was upregulated in African American prostate cancer vs. European American prostate cancer (FIG. 3), which is consistent with the miRNA array data. To further elucidate the functional impacts of these African American-depleted/enriched miRNAs in prostate cancer disparities, four prostate cancer cell lines derived from European American and African American patients were used as in-vitro cell line models. LNCaP and PC-3, lymph node and bone metastasis derived from European American patients respectively, were used as metastatic European American prostate cancer cell models. RC77 T/E, a primary prostate cancer derived from an African American patient, represents a primary African American prostate cancer. While MDA PCa 2b, a bone metastasis derived from an African American prostate cancer patient, was used as a metastatic African American prostate cancer cell model. To verify whether the proposed prostate cancer cell lines can serve as in-vitro European American and African American cell models, RT-qPCR assays of African American-depleted/enriched miRNAs and mRNAs were performed. The RT-qPCR results from these European American and African American prostate cancer cells have again confirmed the microarray data and the RT-qPCR results (FIG. 3) from European American and African American prostate cancer patient samples. Consistent with RT-qPCR results from patient samples, miR-34a-5p and miR-99b-5p were downregulated in African American prostate cancer vs. European American prostate cancer cell lines (FIG. 4A), while their predicted targets PIK3CB, MTOR, HIF1A and IGFBP2 were upregulated in African American prostate cancer vs. European American prostate cancer cells (FIG. 4B). In contrast, RT-qPCR results confirmed that miR-96-5p was upregulated, while the its predicted target MAPKAPK2 was downregulated in African American prostate cancer vs. European American prostate cancer cells (FIG. 4A, FIG. 4B). Western blot analysis was performed to assess the protein levels of mTOR, PI3 KB, HIF1a, IGFBP2 and MAPKAPK2. Immunoblot analysis again confirmed that mTOR, PI3 KB, HIF1a, and IGFBP2 were significantly upregulated, while MAPKAPK2 was downregulated in African American prostate cancer (especially in MDA PCa 2b) in comparison with European American prostate cancer cell lines (FIG. 5A, FIG. 5B). Furthermore, immunoblot analysis was performed to assess the activation status of mTOR and VEGF signaling in European American and African American prostate cancer cells. The Western blot assays have revealed increased phosphorylation states of mTOR and VEGF in the metastatic African American prostate cancer cell line MDA PCa 2b, but not in metastatic European American prostate cancer cell line (LNCaP and PC-3) and primary African American prostate cancer cell line RC77 T/E (FIG. 5C). These results, again, confirmed that mTOR and VEGF signaling pathways are preferentially upregulated in metastatic African American prostate cancer vs. metastatic European American prostate cancer.

Assessment of Regulatory Relationship in Reciprocal miRNA-mRNA Pairings by Modulating African American-Depleted or African American-Enriched miRNA Expression Level

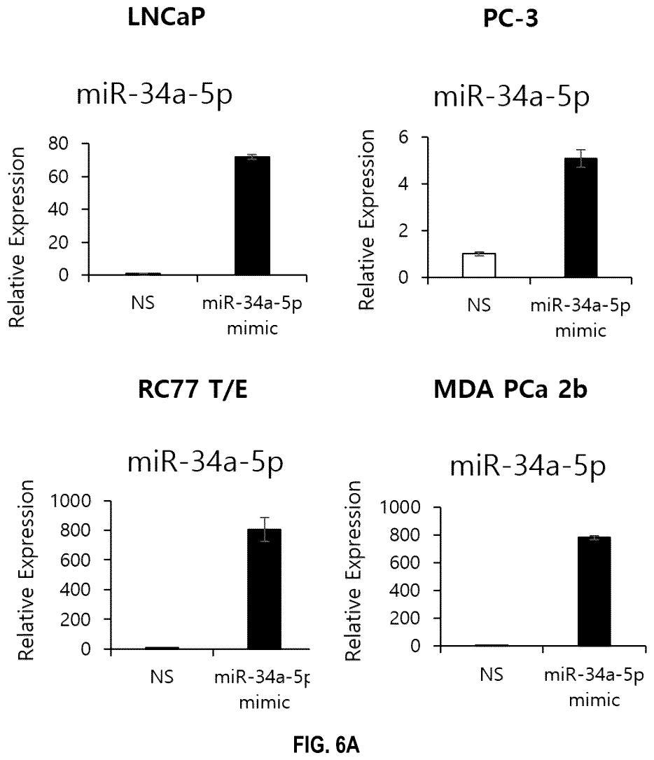

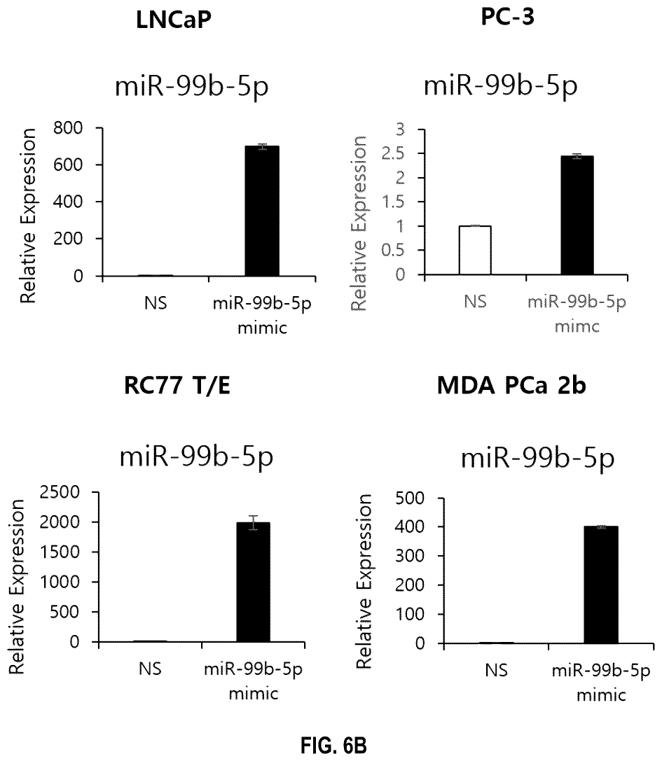

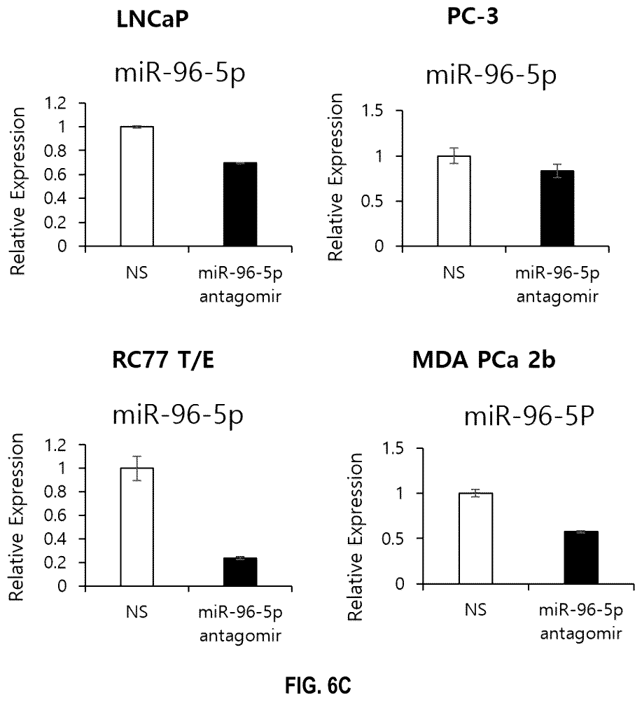

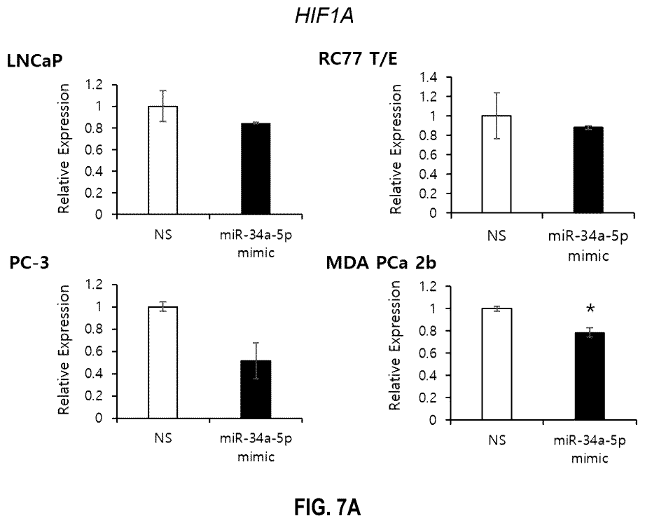

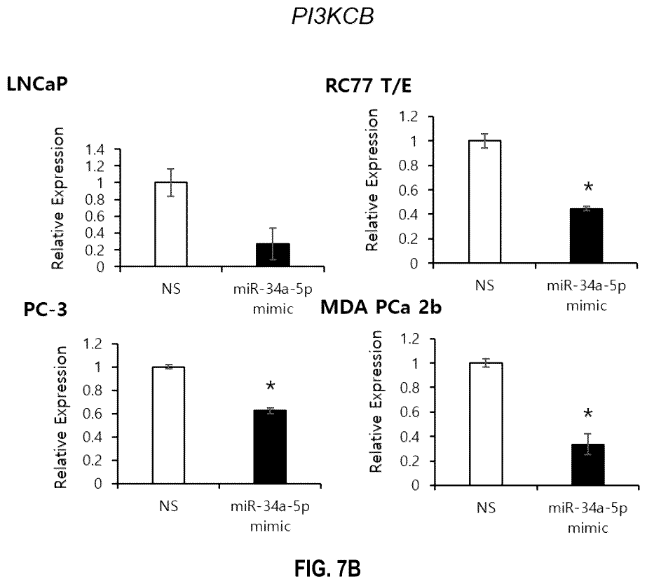

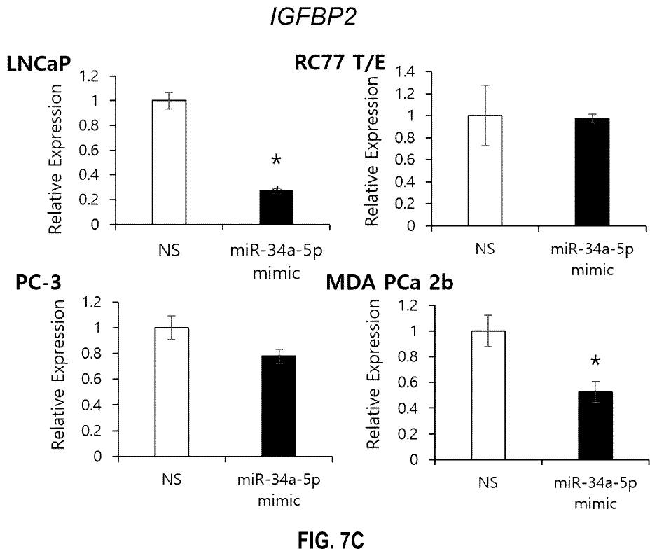

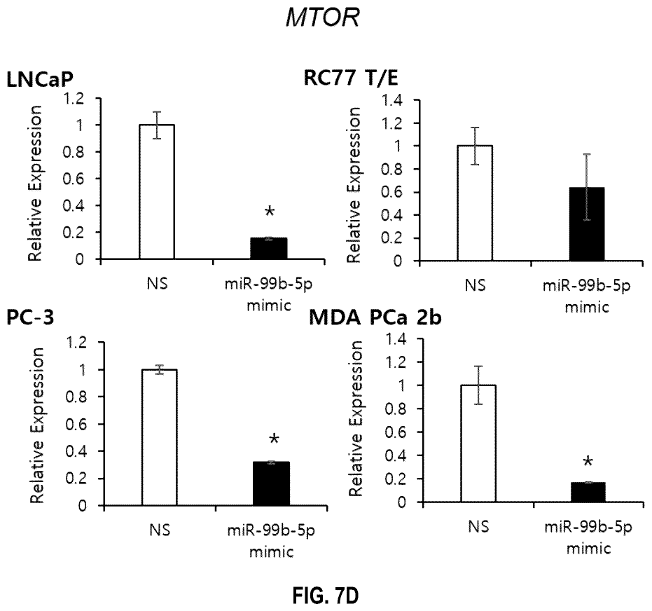

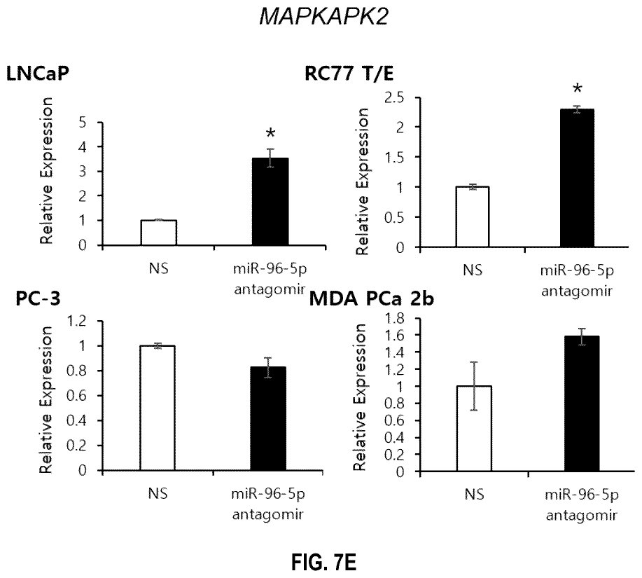

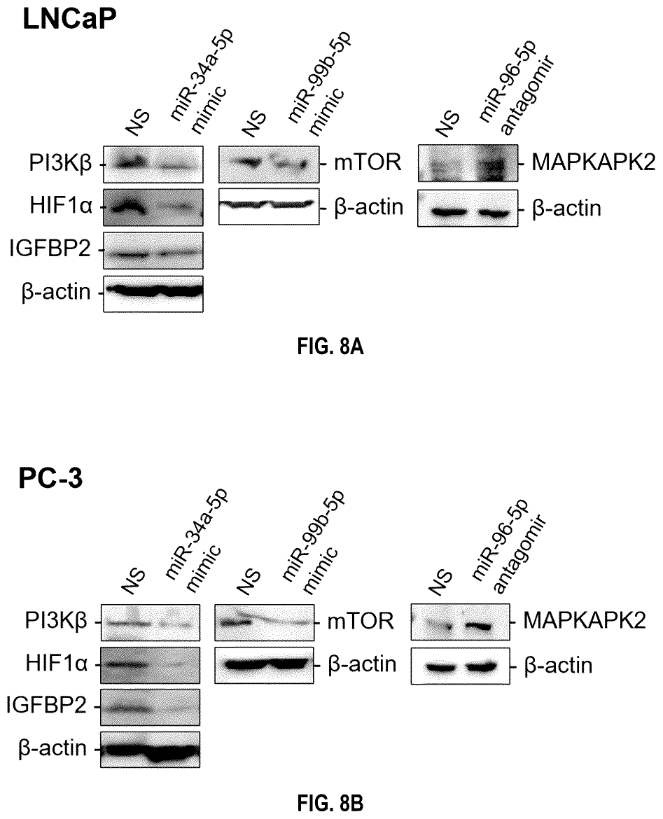

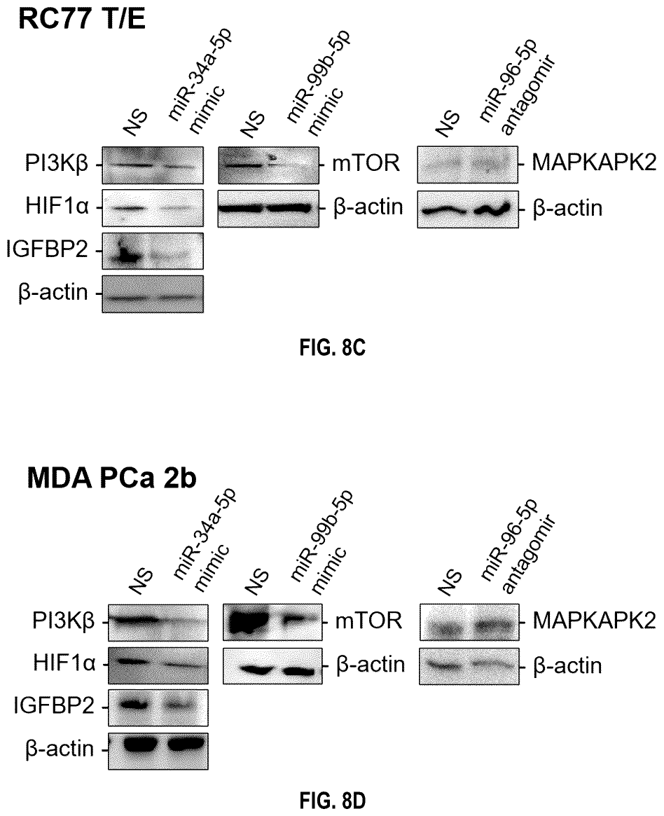

To further verify regulatory relationship in these reciprocal miRNA-mRNA pairings, European American and African American prostate cancer cell lines were transfected with miR-34a-5p mimic, miR-99b-5p mimic or miR-96-5p antagomir then followed by RT-qPCR and Western blot analysis to examine the expression of the predicted miRNA target genes (miR-34a-5p targets genes PIK3CB, HIF1A, and IGFBP2; miR-99b-5p target gene MTOR; and miR-96-5p target gene MAPKAPK2) at mRNA and protein levels. Note that similar transfection efficiencies of miRNA mimics or antagomir in four prostate cancer cell lines were confirmed by RT-qPCR assays (FIG. 6). As anticipated, transfection of miR-34a-5p mimic and miR-99b-5p mimic resulted in decreased PIK3CB, HIF1A, IGFBP2 and MTOR expression in African American and European American prostate cancer cells when compared to prostate cancer cells transfected with nonsense/scrambled (NS) miRNA control (FIG. 7). Transfection of miR-96-5p antagomir increased the expression of MAPKAPK2 transcript in LNCaP, RC77 T/E and MDA PCa 2b cells when compared to the NS-transfected cells (FIG. 7). Since miRNA targeting causes either mRNA degradation or translational repression, the protein levels of mTOR, PI3Kβ, HIF1a, IGFBP2 and MAPKAPK2 in prostate cancer cells transfected with NS, miR-34a-5p mimic, miR-99b-5p mimic, or miR-96-5p antagomir were further investigated. PI3Kβ, HIF1a, and IGFBP2 were downregulated in miR-34a-5p mimic transfected vs. NS transfected prostate cancer cells (FIG. 8). Similarly, mTOR level was downregulated in prostate cancer cells with miR-99b-5p mimic transfection vs. NS transfection. In contrast, MAPKAPK2 was upregulated in miR-96-5p antagomir transfected vs. NS transfected cells (FIG. 8). Taken together, the RT-qPCR and immunoblot assays confirmed that PIK3CB, HIF1A and IGFBP2 expressions are negatively regulated by miR-34a-5p, while MTOR and MAPKAPK2 are negatively regulated by miR-99b-5p and miR-96-5p, respectively.

Modulating Expression Profile of Candidate Reciprocal miRNA-mRNA Pairings Inhibits Cell Proliferation and Sensitizes Docetaxel-Induced Cytotoxicity in Prostate Cancer Cells

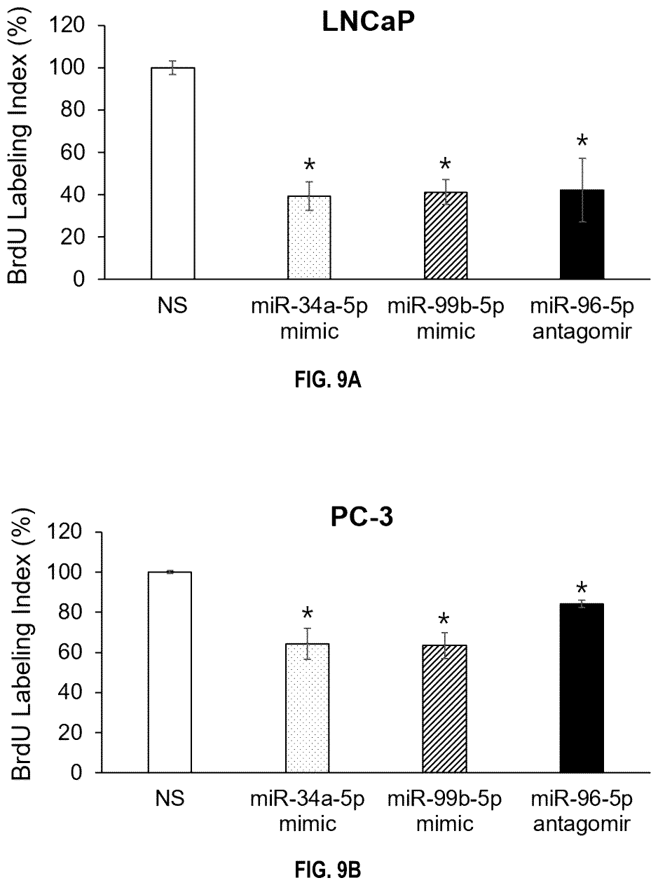

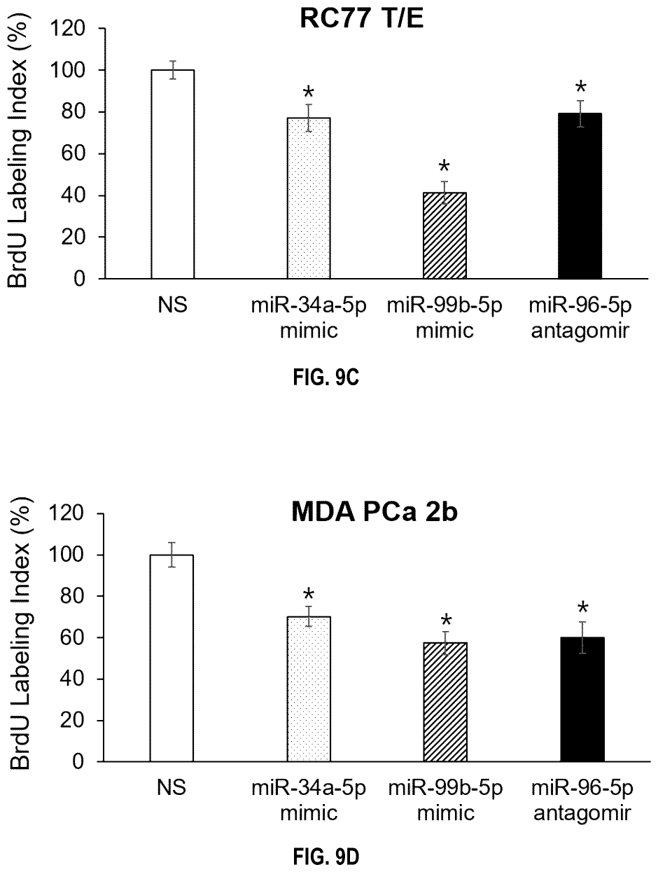

To further test the causal link among the reciprocal miRNA-mRNA pairings identified from mTOR and VEGF signaling in African American prostate cancer (FIG. 1, FIG. 2), miR-34a-5p mimic, miR-99b-5p and miR-96-5p were transfected into European American and African American prostate cancer cells followed by in-vitro functional assays to examine their effects on cell proliferation and apoptosis initiation. Transfection of miR-34a-5p or miR-99b-5p resulted in the reduction of cell proliferation in European American prostate cancer (LNCaP and PC-3) and African American prostate cancer (RC77 T/E and MDA PCa 2b) cell lines (FIG. 9). Conversely, transfection of miR-96-5p antagomir increased the MAPKAPK2 expression (FIG. 8) and subsequently suppressed the cell proliferation in European American and African American prostate cancer cells (FIG. 9).

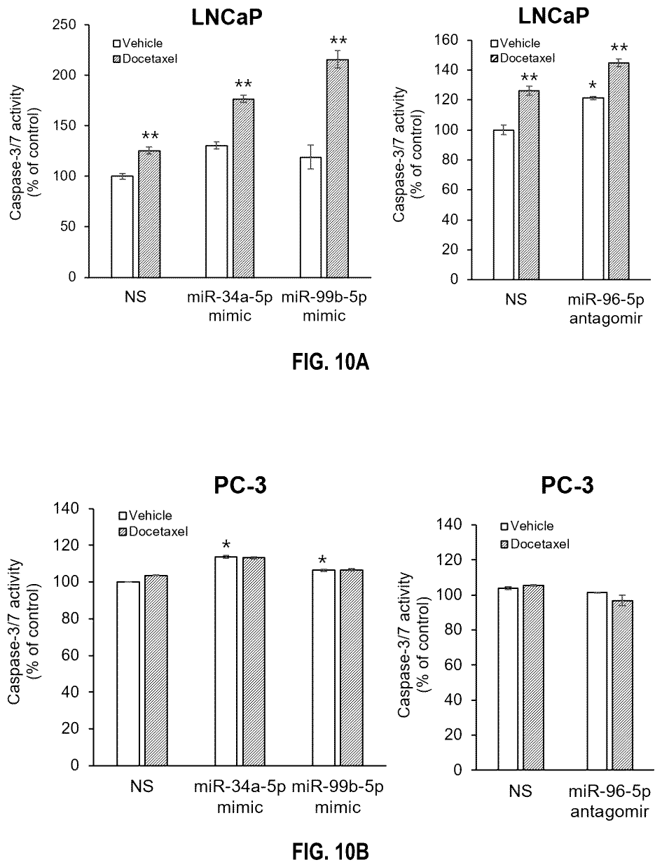

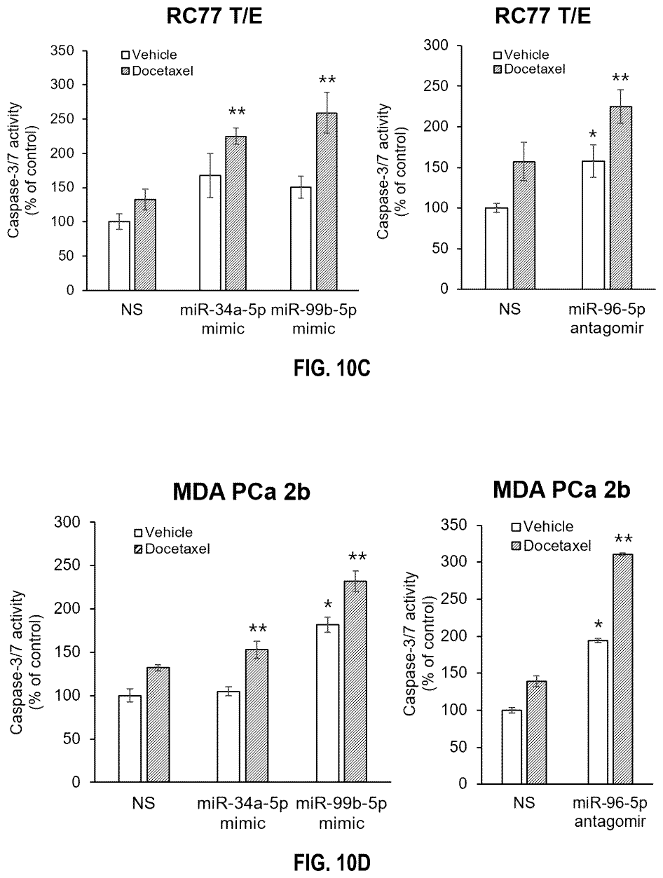

Next, whether these miRNA mimics and antagomir functionally promote cell apoptosis in the absence and presence of docetaxel, a chemotherapeutic agent commonly used in prostate cancer treatment, was tested. Specifically, the European American prostate cancer (LNCaP and PC-3) and African American prostate cancer (RC77 T/E and MDA PCa 2b) cell lines were transfected with NS or miRNA mimic/antagomir in the presence of vehicle or docetaxel treatment, then followed by caspase 3/7 activity assays to measure the apoptosis capacity. In the absence of docetaxel (vehicle control), transfection of prostate cancer cell lines with miR-34a-5p mimic, miR-99b-5p mimic, or miR-96-5p antagomir caused an overall generalized or significant increase (except the miR-96-5p antagomir transfected PC-3) in cell apoptosis compared with NS transfected cells (FIG. 10). Docetaxel treatment alone (without miRNA mimic/antagomir transfection) significantly induced apoptosis in European American cell line LNCaP (FIG. 10A), but not in androgen-independent PC-3 (FIG. 10B) and African American prostate cancer lines (RC77 T/E and MDA PCa 2b, FIG. 10C, FIG. 10D). As anticipated, the combination of docetaxel with miR-34a-5p mimic, miR-99b-5p mimic, or miR-96-5p antagomir further enhanced the docetaxel-induced cytotoxicity in LNCaP cells (FIG. 10A). Interestingly, in the absence of miRNA mimic or antagomir transfection, African American prostate cancer cell lines demonstrated chemoresistance to docetaxel treatment (FIG. 10C, FIG. 10D, docetaxel vs. vehicle in NS transfected cells). In contrast, transfection of miR-34a-5p mimic, miR-99b-5p mimic, or miR-96-5p antagomir in the presence of docetaxel significantly induced apoptosis in the African American cell lines RC77 T/E and MDA PCa 2b (FIG. 10C, FIG. 10D). These results strongly suggest that modulating African American-depleted/enriched miRNAs (i.e., overexpressing miR-34a-5p, miR-99b-5p, or inhibiting miR-96-5p) effectively sensitizes the docetaxel-induced cytotoxicity, especially in African American prostate cancer cells.

### Materials and Methods

Pathway Analysis and Identification of miRNA-mRNA Pairings

DIANA-mirPath V.3 is a web-server based miRNA pathway analysis program that provides accurate statistics by integrating prediction of miRNA targets, based on DIANA-TarBase or TargetScan algorithm, into the identification of significant KEGG pathways. 10 differentially miRNAs in African American prostate cancer vs. European American prostate cancer (TABLE 1) were imported into DIANA-mirPath V.3 program to identify the significant signaling pathways influenced by these miRNAs. Adjusted p-value (FDR<0.05) was applied to identify the most significant KEGG pathways influenced by the miRNAs imported to mirPath V.3 program. The selected signaling pathways, mTOR and VEGF signaling, were further mapped with African American-depleted and -enriched mRNAs (downregulated and upregulated mRNAs in African American prostate cancer vs. European American prostate cancer) to further define the reciprocal miRNA-mRNA pairings in the selected signaling pathways.

### Cell Culture and Transfection

Human prostate cancer cell lines LNCaP and PC-3, derived from lymph node metastasis and bone metastasis of Caucasian patients, and MDA PCa 2b, derived from bone metastasis of an African American patient, were purchased from American Type Culture Collection (ATCC, Manassas, VA). RC77 T/E, a primary prostate cancer cell line derived from an African American patient, was kindly provided by Dr. Johng Rhim at Center for Prostate Disease Research (CPDR) in Rockville, MD. LNCaP and PC-3 were served as European American prostate cancer cell line models, and RC77 T/E and MDA PCa 2b were served as African American prostate cancer cell models in the study. The cells were cultured in specific cell culture media, LNCaP was cultured in RPMI with 10% fetal bovine serum (FBS), PC-3 was cultured in DMEM with 10% FBS, MDA PCa 2b were cultured in BRFF-HPC1 with 20% FBS and RC77 T/E was cultured in Keratinocyte SFM with Human Recombinant Epidermal Growth Factor (EGF 1-53) and Bovine Pituitary Extract (BPE). Cells were maintained at 37° C. in a 5% CO₂ incubator. LNCaP, PC-3 and MDA PCa 2b cells were seeded at density 3×10⁴ cells/well in 6-well plates. RC77 T/E cells were seeded at density 5×10⁴ cells/well in 6-well plates. The cells were allowed to grow for 24 h and then were either transfected with miR-34a-5p mimic, miR-99b-5p mimic, or miR-96-5p antagomir using DharmaFECT4 transfection reagent (Dharmacon), according to the manufacturer's protocol. After 24 h, the cells were replaced with fresh media then incubated for 24 h. miR-34a-5p mimic, miR-99b-5p mimic, miR-96-5p antagomir, and nonsense miRNA mimic and antagomir controls were purchased from Ambion. Nonsense/scrambled miRNA mimic and antagomir were used as negative controls.

RT-qPCR Validation of mRNA and miRNA Expression

For the measurement of gene expression level, total RNA was isolated using miRNeasy Mini Kit (50) (Qiagen) from the prostate cancer cells used in the study. Quantification of total RNA samples were done using NanoVue Plus spectrophotometer (GE Healthcare). For examining mRNA expression level, 1 μg of total RNA was used as template for reverse transcription. Reverse transcription was performed using iScript Reverse Transcription Supermix kit (Bio-Rad) to generate cDNA then followed by performing quantitative PCR (qPCR) assays using SsoAdvanced Universal SYBR Green supermix kit (Bio-Rad). For reverse transcription of miRNAs, the miRNA samples (1 μg per sample) were used in the reverse transcription reactions using Qiagen miScriptII RT kit. Specifically, the miRNAs were poly-A tailed, and reverse transcribed using poly-T primer coupled with 3′-nucleotide code designed by Qiagen. The synthesized cDNAs were mixed with universal reverse primer (Qiagen), specific miRNA primer, and PowerUp SYBR Green master mix (Applied Biosystems) to perform the qPCR reactions.

The qPCR reaction condition for quantification of miRNA expression levels were as follows: pre-denaturation for 5 min at 95° C., followed by 40 standard cycles of: denaturation at 95° C. for 15 s, annealing at 55° C. for 30 s, and extension at 70° C. for 30 s. While the qPCR reaction program for quantification of mRNA level is: pre-denaturation for 5 min at 95° C., followed by 40 standard cycles of: denaturation at 95° C. for 15 s, then annealing and extension at 60° C. for 30 s. Specific primers designed for miR-34a-5p, miR-99b-5p and miR-96-5p and their target genes MTOR, PIK3CB, HIF1A, IGFBP2 and MAPKAPK2 were used for qPCR assays (primer sequences listed in TABLE 3 and TABLE 4). RT-qPCR determination of miRNA and mRNA expression levels were performed in triplicate and normalized to levels of endogenous miR-103a-3p and housekeeping genes EIF1AX, respectively. Normalized gene expression levels were determined using the 2-ACT method.

TABLE 3
Primer sequences for qPCR reactions to examine miRNA
expression levels. Note that the reverse primer for qPCR
assays of miRNAs was the universal primer purchased from Qiagen.

| Mature miRNA ID | Primer sequences (forward, 5′ to 3′) |
|---|---|
| hsa-miR-34a-5p | TGGCAGTGTCTTAGCTGGTTGT |
| | (SEQ ID NO: 4) |
| hsa-miR-96-5p | TTTGGCACTAGCACATTTTTGCT |
| | (SEQ ID NO: 5) |
| hsa-miR-99b-5p | CACCCGTAGAACCGACCTTGCG |
| | (SEQ ID NO: 6) |
| has-103a-3p | AGCAGCATTGTACAGGGCTATGA |
| | (SEQ ID NO: 7) |

TABLE 4
Primer sequences for qPCR reactions to examine mRNA expression levels.

| Gene | Sequences of forward primer | Sequences of reverse primer |
|---|---|---|
| symbol | (5′ to 3′) | (5′ to 3′) |
| PIK3CB | CATGTCAGGGCTGGTCTTTT | GCACTTTTCCAGCTTTCCTG |
| | (SEQ ID NO: 8) | (SEQ ID NO: 9) |
| HIF1A | CCCAATGGATGATGACTTCC | TGGGTAGGAGATGGAGATGC |
| | (SEQ ID NO: 10) | (SEQ ID NO: 11) |
| IGFBP2 | CCCTCAAGTCGGGTATGAAG | ACCTGGTCCAGTTCCTGTTG |
| | (SEQ ID NO: 12) | (SEQ ID NO: 13) |
| MTOR | CCTCACAAGACATCGCTGAA | TCCGGCTGCTGTAGCTTATT |
| | (SEQ ID NO: 14) | (SEQ ID NO: 15) |
| MAPKAPK2 | AGAAGTGCTGGGTCCAGAGA | AATTCATACTGGCCCATTCG |
| | (SEQ ID NO: 16) | (SEQ ID NO: 17) |
| EIF1AX | GTACTGGAGAGGGGAGAGCA | TGAAGCTGAGACAAGCAGGA |
| | (SEQ ID NO: 18) | (SEQ ID NO: 19) |

### Western Blot Analysis

For measurement of the gene expression at protein level, proteins were extracted using M-PER Mammalian Protein Extraction Reagent (Thermo Fisher Scientific) with protease and phosphatase inhibitor cocktail (Thermo Fisher Scientific). Protein concentrations were quantified using BCA assay kit (Thermo Fisher Scientific). Equal amounts of proteins were separated by electrophoresis using Blot 4-12% Bis-Tris gels (Invitrogen) and transferred onto PVDF membranes (Bio-Rad). The PVDF membranes were then incubated with primary antibodies, washed three times with 1×TBST, then incubated with secondary antibodies. Immunoblots were developed with SuperSignal ECL substrate (Thermo Fisher Scientific) and visualized using ChemiDoc XRS imaging system (Bio-Rad). The primary antibodies used in the study were rabbit monoclonal antibodies against mTOR, PI3Kβ, IGFBP2, HIF-1a, MAPKAPK2 and β-actin from Cell Signaling Technology. The secondary antibody used in the study was an anti-rabbit IgG-HRP antibody purchased from Thermo Fisher Scientific.

### BrdU-Labeling Cell Proliferation Assay

LNCaP, PC-3, RC77 T/E and MDA PCa 2b cells were seeded at density 3×10⁴ cells/well in 6-well culture plates. RC77 T/E cells were seeded at density 5×10⁴ cells/well in 6-well culture plates. The cells were incubated for 24 h and then were either transfected with miRNA mimics or antagomir. As a control, cells were cultured with nonsense scrambled control (NS). The cells were incubated for another 24 h. After this period, cells were plated in 96-well plates, bromodeoxyuridine (BrdU) incorporation assay was performed to analyze cell proliferation and cell viability. The assay was conducted using BrdU Cell Proliferation Assay Kit (Sigma-Aldrich) as described by manufacturers. Cells were labelled with BrdU for an incubation period of 8 h in the tissue culture incubator. After incubation, the content of each well was removed and 200 μL of fixative solution was added for 30-min incubation at room temperature. This was followed by adding 100 μL of anti-BrdU antibody into each well, and the cells were further incubated for 1 h at room temperature. The plate was then washed 3 times with 1× wash buffer. 100 μL of goat anti-mouse IgG HRP conjugate was added into each well then incubated for 30 min. After washing three times with 1× wash buffer and additional washing with ddH₂O, 100 μL of substrate solution was added and the cells were incubated in dark for 15 min or until a significant color change was observed. 100 μL of stop solution was used to stop further reaction and followed by checking absorbance at dual wavelengths of 450 nm and 540 nm using Multiskan FC microplate photometer (Thermo Scientific).

### Caspase 3/7 Activity-Based Apoptosis Assay

For the measurement of apoptosis initiation by transfection of miRNA mimic or antagomir in prostate cancer cells, the transfected cells 5000 cells/well were seeded onto 96-well cell culture plates (Corning) and then incubated overnight. The miRNA mimic/antagomir transfected cells were then treated with vehicle or 11 mM of Docetaxel. 24 h after the vehicle or drug treatment, the apoptosis assays were performed using Apo-ONE Caspase-3/7 Assay Kit (Promega Corporation) according to the protocol described by the manufacturer. 100 μL of homogeneous Caspase-3/7 reagent was added to the sample plate and incubated at room temperature for 1 h. Fluorescence was detected for measuring the apoptosis state (Caspase 3/7 activity) using Biotek Synergy HT Microplate Reader (BioTek) at wavelengths of 499 nm and 521 nm for excitation and emission, respectively.

### Discussion

In this study, an integrated genomic approach in combination with a miRNA-driven computational algorithm was utilized to identify the KEGG pathways significantly influenced by the African American-depleted/enriched miRNAs and mRNAs. ErbB-PI3K-AKT-mTOR-HIF1A-VEGF signaling was identified as a critical cascade highly mediated by miRNA-mRNA regulatory network in African American prostate cancer. A novel panel of reciprocal miRNA-mRNA pairings (miR-34a-5p/HIF1A, miR-34a-5p/IGFBP2, miR-34a-5p/PIK3CB, miR-99b-5p/MTOR, and miR-96-5p/MAPKAPK2) were identified as core regulators contributing to the upregulation of mTOR and VEGF signaling in African American prostate cancer. By applying molecular and biochemical approaches, it was confirmed that HIF1A, IGFBP2 and PIK3CB is negatively regulated by miR-34a-5p, while MTOR and MAPKAPK2 are negatively regulated by miR-99b-5p and miR-96-5p, respectively. Furthermore, functional assays validated that modulating expression profile of miR-34a-5p/HIF1A, miR-34a-5p/IGFBP2, miR-34a-5p/PIK3CB, miR-99b-5p/MTOR, or miR-96-5p/MAPKAPK2 using miRNA mimics/antagomir subsequently inhibits cell proliferation and promotes docetaxel-induced cytotoxicity in prostate cancer cells. Especially, African American prostate cancer cells (more resistant to docetaxel treatment than European American prostate cancer cells) demonstrated higher sensitivity to the treatment when combining miRNA mimics/antagomir (miR-34a-5p mimic, miR-99b-5p mimic, or miR-96-5p) with docetaxel. These results suggest that targeting these reciprocal miRNA-mRNA pairings may overcome chemoresistance and greatly sensitizes docetaxel-induced cytotoxicity, potentially serving as novel synergistic therapeutics for treating African American prostate cancer.

Previous studies have demonstrated the critical functional roles of miR-34a-5p and miR-99b-5p as tumor suppressive miRNAs, and miR-96-5p as an oncogenic miRNA in various types of cancers. Downregulation of miR-34a-5p has been implicated in pancreatic cancer, hepatocellular carcinoma (HCC), and prostate cancer. Furthermore, overexpression of miR-34a-5p resulted in significant decrease in cell proliferation and migration, and significant enhancement of cell apoptosis in HCC and prostate cancer cells. Belonging to miR-125a-let-7e cluster family, miR-99b-5p has been reported to play a role in cell proliferation, migration, and differentiation in tumor cells. A previous study showed a significant reduction of miR-99b-5p expression in osteosarcoma (OS) tissue/cell lines compared to normal tissues/cells, suggesting it could potentially serve as a biomarker for OS patients. MiR-96-5p belongs to the cluster miR-183-96-182 family and has been found upregulated in various cancers including breast cancer, thyroid cancer, bladder cancer, adrenocortical and adrenal medullary tumors, head, neck squamous cell carcinoma, and cervical cancer. Furthermore, high-level expression of miR-96-5p is correlated with a poor overall survival rate of prostate cancer patients, and has been implicated in promoting prostate cancer growth, proliferation and tumor progression.

Hypoxia-inducible factors (HIFs) act as a driving force for cancer cells to adapt hypoxic condition, which contributes to cancer progression and treatment resistance. Previous study further revealed androgen receptor (AR)-hypoxia-HIF1a axis as an independent pathway to promote prostate cancer development. It has been reported that HIF1A is a direct mRNA target of miR-34a-5p, and overexpression of miR-34a-5p downregulates HIF1a and other epithelial-to-mesenchymal transition (EMT) markers, consequently inhibiting VEGFR signaling in breast cancer. Another study also indicated that miR-34a-5p directly targets HIF1A and inhibits its transcription, preventing PPP1R11/STAT3-induced EMT and metastasis in colorectal cancer (CRC). This study is the first report to confirm that transcription and translation of HIF1A is also negatively regulated by miR-34a-5p in prostate cancer (FIG. 7, FIG. 8). Taken together, p53-miR-34a-5p-HIF1a signaling axis may play a critical role in pathogenesis of prostate cancer and other cancers. Therefore, targeting miR-34a-5p-HIF1A pairing (i.e., using miR-34a-5p mimic or HIF1a inhibitor) may serve as an effective molecular strategy to reduce prostate cancer aggressiveness, especially in African American patients.

Insulin-like growth factor binding protein 2 (IGFBP2) is an oncogenic protein involved in the development and progression of various cancers, such as glioma, breast, lung and prostate cancers. In addition, IGFBP2 has been shown to overexpress in prostate cancer and prostatic intraepithelial neoplasia (PIN) tissues. Prostate cancer cells produce significantly high amount of IGFBP2, and the serum level of IGFBP2 is positively correlated with the tumor grades and stages, suggesting the potential role of IGFBP2 as a biomarker in prostate cancer risk, resistance and relapse. Furthermore, IGFBP2 has been uncovered as a direct target of miR-34a-5p, and miR-34a-5p expression inhibits the expression of IGFBP2 at transcriptional and translational levels in myoblasts. The results also confirmed, as the first time, that IGFBP2 is negatively regulated by miR-34a-5p at transcriptional and translation levels in prostate cancer cells (FIG. 7, FIG. 8). These results suggest miR-34a-5p/IGFBP2 pairing as a potential precision biomarker and novel drug target in prostate cancer.

Excessive activity in the PI3K/AKT/mTOR signaling pathway, one of the most common hallmarks in human cancers, is an important therapeutic target for cancer treatment. Aberration of p110β (PI3Kβ) and its overexpression has been implicated in carcinogenesis process in different cancers. Previous study revealed that miR-34a-5p directly targets/inhibits PIK3CB and participates in the regulation of TCR-mediated NFκB signaling in CD4⁺ and CD8⁺ T cells. Similarly, the data confirmed, as the first time, that PIK3CB is negatively regulated by miR-34a-5p at mRNA and protein levels in prostate cancer (FIG. 7, FIG. 8), indicating miR-34a-5p/PIK3CB reciprocal pairing as a potential biomarker as well as novel therapeutic target in African American prostate cancer.

mTOR is a downstream target of PI3K/AKT survival pathways and functions as a regulator involved in cell growth, proliferation, and survival. Deregulation of mTOR contributes to cancer progression and drug resistance. MiR-99a/b has been implicated as a tumor suppressor frequently downregulated in human cancers, and plays an important role in regulating mTOR signaling pathway. Several studies have shown that downregulation of miR-99b-5p is correlated with the elevated levels of mTOR in prostate cancer and endometrial carcinoma. Previous study has further revealed that miR-99b-5 expression suppresses mRNA and protein levels of mTOR, PI3K, AKT and p70S6, thereby inhibiting PI3K/AKT/mTOR signaling in human cervical cancer. Upregulation of miR-99b-5p results in a longer survival, while silencing of miR-99b-5p causes upregulation of mTOR and promotes cell migration in CRC. MiR-99b-5p has been revealed to directly or indirectly target MTOR, and inhibiting miR-99b-5p expression causes upregulation of mTOR in prostate cancer and endometrial carcinoma. In addition, overexpression of miR-99a/b in HCC cells results in significant inhibition of tumor growth by suppressing the expression of IGF1R and MTOR. In this study, it was confirmed that miR-99b-5p functions as a tumor suppressive miRNA (FIG. 9, FIG. 10), and its expression contributes to the suppression of MTOR expression at transcriptional and translational levels in prostate cancer (FIG. 7, FIG. 8). Given all, deregulation of miR-99-MTOR signaling represents a crucial miRNA-mRNA interaction in prostate cancer development/progression. Modulation of miR-99b-5p/MTOR expression profile through miR-99b-5p mimic reduces the prostate cancer aggressiveness and sensitizes docetaxel-induced cytotoxicity, especially in African American prostate cancer.

Activation of the pathway mitogen-activated protein kinase (MAPK) is found to play a diverse role in multiple cellular mechanisms, including cell growth, migration, proliferation, differentiation, and apoptosis. Mitogen-activated protein kinase 2 (MAPKAPK2, also named as MK2) is a direct downstream substrate of p38 MAPK, and it plays a critical role in regulating particular cellular functions, including apoptosis, cell cycle, DNA repair, RNA metabolism, autophagy, inflammation, post-translational regulation of gene expression, and stress response to oxidative agents. Previous studies have shown that p38 MAPK has a dual role as a tumor suppressor kinase or a tumor promoter, however, the diverse functional roles of MAPKAPK2 remain elusive. Previous study has implicated MAPKAPK2 as an oncogene involved in tumorigenesis in lung, colorectal, skin, bladder, and prostate cancers. Several reports have also demonstrated that p38 MAPK signaling functions as an antitumor pathway. p38 MAPK exerts its tumor suppressive activities by inhibiting oncogenic transformation, such as regulating cell cycle, inhibiting cell proliferation, activating cell apoptosis, inducing senescence, modulating inflammatory-dependent transformation, and promoting cell differentiation. MAPKAPK2, a key p38 downstream substrate involved in cell cycle control, DNA repair, immune response, senescence, and autophagy, may also function as a cofactor in the p38-mediated tumor suppressor pathways. In this study, DIANA-TarBase algorithm predicted MAPKAPK2 as a target of miR-96-5p, and the results have further confirmed that miR-96-5p negatively regulates MAPKAPK2 expression at transcriptional and translational levels. This is the first study to confirm that MAPKAPK2 expression is mediated by miR-96-5p. Further luciferase reporter assay will validate whether MAPKAPK2 is a direct or indirect miR-96-5p target. In summary, although the tumor suppressive role of MAPKAPK2 remains elusive, the results have implicated miR-96-5p/MAPKAPK2 as a promising therapeutic target for reducing the chemoresistance in African American prostate cancer.

In conclusion, this study revealed that EGFR-PI3K-ATK-mTOR-HIF1α-VEGF axis is preferentially upregulated by African American-depleted/enriched miRNAs in prostate cancer. The novel panel of reciprocal miRNA-mRNA pairings within mTOR signaling (miR-34a-5p/HIF1A, miR-34a-5p/IFGBP2, and miR-99b-5p/MTOR) and VEGF signaling (miR-34a-5p/PIK3CB, and miR-96-5p/MAPKAPK2) have been suggested as critical miRNA-mRNA regulatory components in African American prostate cancer (or more aggressive type of prostate cancer). Further exploring a computational algorithm using combined expression profiles of these miRNA/mRNA pairings may facilitate the development of precision diagnostic/prognostic biomarkers in prostate cancer. Finally, targeting miRNA-mRNA reciprocal pairings (i.e., using miRNA mimics/antagomirs, siRNAs, antisense oligonucleotides, or Crispr knockdown) may lead to the development of novel molecular strategies to overcome chemoresistance observed in aggressive African American prostate cancer.

### REFERENCES

Abramovic, I.; Ulamec, M.; Bojanac, A. K.; Bulic-Jakus, F.; Jezek, D.; Sincic, N. miRNA in prostate cancer: Challenges toward translation. Epigenomics 2020, 12, 543-558.
Beltran, H.; Morris, M. J. Docetaxel for Early Prostate Cancer: What Have We Learned? Eur. Urol. 2020, 77, 573-575.
Canovas, B.; Nebreda, A. R. Diversity and versatility of p38 kinase signalling in health and disease. Nat. Rev. Mol. Cell Biol. 2021, 22, 346-366.
Chen, B.; Li, H.; Zeng, X.; Yang, P.; Liu, X.; Zhao, X.; Liang, S. Roles of microRNA on cancer cell metabolism. J. Transl. Med. 2012, 10, 228.
Cheng, H.; Xue, J.; Yang, S.; Chen, Y.; Wang, Y.; Zhu, Y.; Wang, X.; Kuang, D.; Ruan, Q.; Duan, Y.; et al. Co-targeting of IGF1R/mTOR pathway by miR-497 and miR-99a impairs hepatocellular carcinoma development. Oncotarget 2017, 8, 47984-47997.
Chua, C. Y.; Liu, Y.; Granberg, K. J.; Hu, L.; Haapasalo, H.; Annala, M. J.; Cogdell, D. E.; Verploegen, M.; Moore, L. M.; Fuller, G.; et al. IGFBP2 potentiates nuclear EGFR-STAT3 signaling. Oncogene 2015, 35, 738-747.
Dbouk, H. A.; Pang, H.; Fiser, A.; Backer, J. M. A biochemical mechanism for the oncogenic potential of the p110beta catalytic subunit of phosphoinositide 3-kinase. Proc. Natl. Acad. Sci. USA 2010, 107, 19897-19902.
Dong, B.; Xu, G.-C.; Liu, S.-T.; Liu, T.; Geng, B. MiR-34a affects G2 arrest in prostate cancer PC3 cells via Wnt pathway and inhibits cell growth and migration. Eur. Rev. Med. Pharmacol. Sci. 2020, 24, 8349-8358.
Evans, S.; Metcalfe, C.; Ibrahim, F.; Persad, R.; Ben-Shlomo, Y. Investigating Black-White differences in prostate cancer prognosis: A systematic review and meta-analysis. Int. J. Cancer 2008, 123, 430-435.
Freedland, S. J.; Isaacs, W. B. Explaining racial differences in prostate cancer in the United States: Sociology or biology? Prostate 2004, 62, 243-252.
Friedman, R. C.; Farh, K. K.-H.; Burge, C. B.; Bartel, D. P. Most mammalian mRNAs are conserved targets of microRNAs. Genome Res. 2008, 19, 92-105.
Ha, M.; Kim, V. N. Regulation of microRNA biogenesis. Nat. Rev. Mol. Cell Biol. 2014, 15, 509-524.
Haflidadóttir, B. S.; Larne, O.; Martin, M.; Persson, M.; Edsjö, A.; Bjartell, A.; Ceder, Y. Upregulation of miR-96 Enhances Cellular Proliferation of Prostate Cancer Cells through FOXO1. PLOS ONE 2013, 8, c72400.
Hart, M.; Walch-Rückheim, B.; Friedmann, K. S.; Rheinheimer, S.; Tänzer, T.; Glombitza, B.; Sester, M.; Lenhof, H.-P.; Hoth, M.; Schwarz, E. C.; et al. miR-34a: A new player in the regulation of T cell function by modulation of NF-kappaB signaling. Cell Death Dis. 2019, 10, 46.
Hashimoto, Y.; Shiina, M.; Dasgupta, P.; Kulkarni, P.; Kato, T.; Wong, R. K.; Tanaka, Y.; Shahryari, V.; Mackawa, S.; Yamamura, S.; et al. Upregulation of miR-130b Contributes to Risk of Poor Prognosis and Racial Disparity in African-American Prostate Cancer. Cancer Prev. Res. 2019, 12, 585-598.
Houede, N.; Pourquier, P. Targeting the genetic alterations of the PI3K-AKT-mTOR pathway: Its potential use in the treatment of bladder cancers. Pharmacol. Ther. 2015, 145, 1-18.
Jackson, R. J.; Standart, N. How do microRNAs regulate gene expression? Sci. STKE 2007, 2007, rel.
Jiang, B.-H.; Liu, L.-Z. Role of mTOR in anticancer drug resistance: Perspectives for improved drug treatment. Drug Resist. Updates 2008, 11, 63-76.
Lewis, B. P.; Burge, C. B.; Bartel, D. P. Conserved Seed Pairing, Often Flanked by Adenosines, Indicates that Thousands of Human Genes are MicroRNA Targets. Cell 2005, 120, 15-20.
Li, H.; Rokavec, M.; Jiang, L.; Horst, D.; Hermeking, H. Antagonistic Effects of p53 and HIF1A on microRNA-34a Regulation of PPP1R11 and STAT3 and Hypoxia-induced Epithelial to Mesenchymal Transition in Colorectal Cancer Cells. Gastroenterology 2017, 153, 505-520.
Li, W.; Chang, J.; Wang, S.; Liu, X.; Peng, J.; Huang, D.; Sun, M.; Chen, Z.; Zhang, W.; Guo, W.; et al. miRNA-99b-5p suppresses liver metastasis of colorectal cancer by down-regulating mTOR. Oncotarget 2015, 6, 24448-24462.
Li, Y. J.; Wang, Y.; Wang, Y. Y. MicroRNA-99b suppresses human cervical cancer cell activity by inhibiting the PI3K/AKT/mTOR signaling pathway. J. Cell Physiol. 2019, 234, 9577-9591.
Liu, B.; Yang, L.; Huang, B.; Cheng, M.; Wang, H.; Li, Y.; Huang, D.; Zheng, J.; Li, Q.; Zhang, X.; et al. A Functional Copy-Number Variation in MAPKAPK2 Predicts Risk and Prognosis of Lung Cancer. Am. J. Hum. Genet. 2012, 97, 384-390.
Liu, B.; Zhang, J.; Yang, D. miR-96-5p promotes the proliferation and migration of ovarian cancer cells by suppressing Caveolae1. J. Ovarian Res. 2019, 12, 57.
Lu, J.; Getz, G.; Miska, E. A.; Alvarez-Saavedra, E.; Lamb, J.; Peck, D.; Swect-Cordero, A.; Ebert, B. L.; Mak, R. H.; Ferrando, A. A.; et al. MicroRNA expression profiles classify human cancers. Nature 2005, 435, 834-838.
Ma, X.; Shi, W.; Peng, L.; Qin, X.; Hui, Y. MiR-96 enhances cellular proliferation and tumorigenicity of human cervical carcinoma cells through PTPN9. Saudi J. Biol. Sci. 2018, 25, 863-867.
Mansoori, B.; Silvestris, N.; Mohammadi, A.; Khaze, V.; Baghbani, E.; Mokhtarzadch, A.; Shanchbandi, D.; Derakhshani, A.; Duijf, P. H. G.; Baradaran, B. miR-34a and miR-200c Have an Additive Tumor-Suppressive Effect on Breast Cancer Cells and Patient Prognosis. Genes 2021, 12, 267.
Martínez-Limón, A.; Joaquin, M.; Caballero, M.; Posas, F.; De Nadal, E. The p38 Pathway: From Biology to Cancer Therapy. Int. J. Mol. Sci. 2020, 21, 1913.
Michlewski, G.; Cáceres, J. F. Post-transcriptional control of miRNA biogenesis. RNA 2018, 25, 1-16.
Nalls, D.; Tang, S.-N.; Rodova, M.; Srivastava, R. K.; Shankar, S. Targeting Epigenetic Regulation of miR-34a for Treatment of Pancreatic Cancer by Inhibition of Pancreatic Cancer Stem Cells. PLOS ONE 2011, 6, c24099.
Ozen, M.; Creighton, C. J.; Ozdemir, M.; Ittmann, M. Widespread deregulation of microRNA expression in human prostate cancer. Oncogene 2007, 27, 1788-1793.
Paccez, J. D.; Duncan, K.; Sekar, D.; Correa, R. G.; Wang, Y.; Gu, X.; Bashin, M.; Chibale, K.; Libermann, T. A.; Zerbini, L. F. Dihydroartemisinin inhibits prostate cancer via JARID2/miR-7/miR-34a-dependent downregulation of Ax1. Oncogenesis 2019, 8, 14.
Pedroza-Torres, A.; Romero-Córdoba, S. L.; Justo-Garrido, M.; Salido-Guadarrama, I.; Rodríguez-Bautista, R.; Montaño, S.; Muñiz-Mendoza, R.; Arriaga-Canon, C.; Fragoso-Ontiveros, V.; Álvarez-Gómez, R. M.; et al. MicroRNAs in Tumor Cell Metabolism: Roles and Therapeutic Opportunities. Front. Oncol. 2019, 9, 1404.
Pelka, K.; Klicka, K.; Grzywa, T. M.; Gondek, A.; Marczewska, J. M.; Garbicz, F.; Szczepaniak, K.; Paskal, W.; Włodarski, P. K. miR-96-5p, miR-134-5p, miR-181b-5p and miR-200b-3p heterogenous expression in sites of prostate cancer versus benign prostate hyperplasia-archival samples study. Histochem. Cell Biol. 2021, 155, 423-433.
Powell, I. J. Epidemiology and Pathophysiology of Prostate Cancer in African-American Men. J. Urol. 2007, 177, 444-449.
Ramalho-Carvalho, J.; Fromm, B.; Henrique, R.; Jerónimo, C. Deciphering the function of non-coding RNAs in prostate cancer. Cancer Metastasis Rev. 2016, 35, 235-262.
Reddy, S.; Shapiro, M.; Morton, J. R.; Brawley, O. W. Prostate cancer in black and white Americans. Cancer Metastasis Rev. 2003, 22, 83-86.
Richardsen, E.; Ukkonen, T.; Bjørnsen, T.; Mortensen, E.; Egevad, L.; Busch, C. Overexpression of IGBFB2 is a marker for malignant transformation in prostate epithelium. Virchows Arch. 2003, 442, 329-335.
Robbins, A. S.; Whittemore, A. S.; Thom, D. H. Differences in socioeconomic status and survival among white and black men with prostate cancer. Am. J. Epidemiol. 2000, 151, 409-416.
Russo, V.; Azar, W.; Yau, S.; Sabin, M.; Werther, G. IGFBP-2: The dark horse in metabolism and cancer. Cytokine Growth Factor Rev. 2014, 26, 329-346.
Shi, X.; Guan, X. MicroRNA-99b predicts clinical outcome of osteosarcoma and suppresses tumor cell proliferation, migration and invasion. Diagn. Pathol. 2019, 14, 117.
Shiina, M.; Hashimoto, Y.; Kato, T.; Yamamura, S.; Tanaka, Y.; Majid, S.; Saini, S.; Varahram, S.; Kulkarni, P.; Dasgupta, P.; et al. Differential expression of miR-34b and androgen receptor pathway regulate prostate cancer aggressiveness between African-Americans and Caucasians. Oncotarget 2016, 8, 8356-8368.
Shiina, M.; Hashimoto, Y.; Kulkarni, P.; Dasgupta, P.; Shahryari, V.; Yamamura, S.; Tanaka, Y.; Dahiya, R. Role of miR-182/PDCD4 axis in aggressive behavior of prostate cancer in the African Americans. BMC Cancer 2021, 21, 1028.
Siegel, R. L.; Miller, K. D.; Fuchs, H. E.; Jemal, A. Cancer Statistics, 2021. CA Cancer J. Clin. 2021, 71, 7-33.
Soni, S.; Anand, P.; Padwad, Y. S. MAPKAPK2: The master regulator of RNA-binding proteins modulates transcript stability and tumor progression. J. Exp. Clin. Cancer Res. 2019, 38, 121.
Stevenson, M.; Banerjee, N. N.; Banerjee, H. N.; Rawat, K.; Chen, L.; Worthington, M.; Hodge, S.; Walker, R.; Verma, M.; Sarkar, F.; et al. A health disparities study of MicroRNA-146a expression in prostate cancer samples derived from African American and European American patients. J. Solid Tumors 2020, 10.
Suzuki, H.; Maruyama, R.; Yamamoto, E.; Kai, M. Epigenetic alteration and microRNA dysregulation in cancer. Front. Genet. 2013, 4, 258.
Tomari, Y.; Zamore, P. D. MicroRNA Biogenesis: Drosha Can't Cut It without a Partner. Curr. Biol. 2005, 15, R61-R64.
Torres, A.; Torres, K.; Pesci, A.; Ceccaroni, M.; Paszkowski, T.; Cassandrini, P.; Zamboni, G.; Maciejewski, R. Deregulation of miR-100, miR-99a and miR-199b in tissues and plasma coexists with increased expression of mTOR kinase in endometrioid endometrial carcinoma. BMC Cancer 2012, 12, 369-369.
Tran, M. G. B.; Bibby, B. A. S.; Yang, L.; Lo, F.; Warren, A. Y.; Shukla, D.; Osborne, M.; Hadfield, J.; Carroll, T.; Stark, R.; et al. Independence of HIF1a and androgen signaling pathways in prostate cancer. BMC Cancer 2020, 20, 469.
Tsai, T.-F.; Lin, J.-F.; Chou, K.-Y.; Lin, Y.-C.; Chen, H.-E.; Hwang, T. I.-S. miR-99a-5p acts as tumor suppressor via targeting to mTOR and enhances RAD001-induced apoptosis in human urinary bladder urothelial carcinoma cells. OncoTargets Ther. 2018, 11, 239-252.
Vlachos, I. S.; Zagganas, K.; Paraskevopoulou, M. D.; Georgakilas, G.; Karagkouni, D.; Vergoulis, T.; Dalamagas, T.; Hatzigeorgiou, A. G. DIANA-miRPath v3.0: Deciphering microRNA function with experimental support. Nucleic Acids Res. 2015, 43, W460-W466.
Volinia, S.; Calin, G. A.; Liu, C. G.; Ambs, S.; Cimmino, A.; Petrocca, F.; Visone, R.; Iorio, M.; Roldo, C.; Ferracin, M.; et al. A microRNA expression signature of human solid tumors defines cancer gene targets. Proc. Natl. Acad. Sci. USA 2006, 103, 2257-2261.
Wang, B.-D.; Ceniccola, K.; Yang, Q.; Andrawis, R.; Patel, V.; Ji, Y.; Rhim, J.; Olender, J.; Popratiloff, A.; Latham, P.; et al. Identification and Functional Validation of Reciprocal microRNA-mRNA Pairings in African American Prostate Cancer Disparities. Clin. Cancer Res. 2015, 27, 4970-4984.
Wang, B.-D.; Yang, Q.; Ceniccola, K.; Bianco, F.; Andrawis, R.; Jarrett, T.; Frazier, H.; Patierno, S. R.; Lec, N. H. Androgen Receptor-Target Genes in African American Prostate Cancer Disparities. Prostate Cancer 2013, 2013, 763569.
Wang, Z.; Zhang, X.; Li, Z.; Abdalla, B. A.; Chen, Y.; Nic, Q. MiR-34b-5p Mediates the Proliferation and Differentiation of Myoblasts by Targeting IGFBP2. Cells 2019, 8, 360.
Wang, Z.; Zhao, Z.; Yang, Y.; Luo, M.; Zhang, M.; Wang, X.; Liu, L.; Hou, N.; Guo, Q.; Song, T.; et al. MiR-99b-5p and miR-203a-3p Function as Tumor Suppressors by Targeting IGF-1R in Gastric Cancer. Sci. Rep. 2018, 8, 10119.
Wei, F.; Liu, Y.; Guo, Y.; Xiang, A.; Wang, G.; Xue, X.; Lu, Z. miR-99b-targeted mTOR induction contributes to irradiation resistance in pancreatic cancer. Mol. Cancer 2013, 12, 81.
Yu, T.; Tang, B.; Sun, X. Development of Inhibitors Targeting Hypoxia-Inducible Factor 1 and 2 for Cancer Therapy. Yonsei Med. J. 2017, 58, 489-496.
Yue, J.; López, J. M. Understanding MAPK Signaling Pathways in Apoptosis. Int. J. Mol. Sci. 2020, 27, 2346.
Zhang, Y. M.; Wu, Q. M.; Chang, L. Y.; Liu, J. C. miR-34a and miR-125a-5p inhibit proliferation and metastasis but induce apoptosis in hepatocellular carcinoma cells via repressing the MACC1-mediated PI3K/AKT/mTOR pathway. Neoplasma 2020, 67, 1042-1053.

### Example 2: Downregulation of miR-99b-5p and Upregulation of Nuclear mTOR Cooperatively Promotes the Tumor Aggressiveness and Drug Resistance in African American Prostate Cancer

Mammalian target of rapamycin (mTOR) regulates a wide range of cellular events including cell proliferation, protein translation, metabolism, regeneration, autophagy, and apoptosis. In the context of molecular mechanism, mTOR plays a central role coordinating ERBB (also known as EGFR/PI3K/AKT) and VEGF signaling in the ERBB/mTOR/VEGF axis, a signaling network frequently upregulated in prostate cancer. This study particularly focused on the reciprocal pairing miR-99b-5p/MTOR, where miR-99b-5p is downregulated while MTOR is upregulated in African American prostate cancer compared to European American prostate cancer. To further explore the functional impacts of such miRNA-mRNA pairing in prostate cancer disparities and to evaluate whether miR-99b-5p/MTOR (down/up) could potentially serve as a precision biomarker for aggressive prostate cancer and other advanced cancers, a series of pathological, cellular/molecular biology and biochemical experiments were performed. First, immunohistochemistry (IHC) assays were conducted on tissue microarrays (TMAs) to examine the expression levels of mTOR in prostate cancer specimens derived from European American and African American patients, and specimens derived from prostate, breast, colon, and lung cancer patients. Second, Western blot and RT-qPCR assays were performed to examine the expression levels of mTOR and miR-99b-5p, respectively, in both normal and cancerous cell lines derived from prostate, breast, colon and lung. Third, immunofluorescence assays were followed to investigate the expression levels and subcellular distributions of mTOR and its active form phosphorylated mTOR (pmTOR) in prostate, colon, breast, and lung cancer cell lines. Finally, an miR-99b-5p mimic was transfected to a panel of prostate, breast, colon, and lung cancer cell lines. The functional impacts of miR-99b-5p on regulating mTOR expression and cellular locations, cell apoptosis, and cytotoxic chemotherapy in these cancer cell lines were assessed by using immunofluorescence, Western blot, TUNEL, and apoptosis assays. Briefly, this study aimed to evaluate the potential of reciprocal miR-99b-5p/mTOR (down/up) as a novel diagnostic/prognostic biomarker, and to explore the plausible functional roles of miR-99b-5p in regulating cellular mTOR dynamics and the mTOR-mediating downstream signaling in prostate cancer aggressiveness/progression. In summary, it was demonstrated that mTOR is highly expressed while miR-99b-5p is downregulated in prostate cancer and other cancers. In addition, elevated nuclear mTOR expression were observed in African American prostate cancer and advanced cancer cells. These results suggest that miR-99b-5p/nuclear mTOR may serve as a potential diagnostic/prognostic biomarker for aggressive prostate cancer and other cancers. The functional assays have further implied that the miR-99b-5p-mediated AR/mTOR axis and nuclear mTOR expression/translocation may play critical functional roles for determining the prostate cancer aggressiveness.

### Results

Upregulation of mTOR in African American Prostate Cancer Compared to European American Prostate Cancer, and in Prostate Cancer Compared to Normal

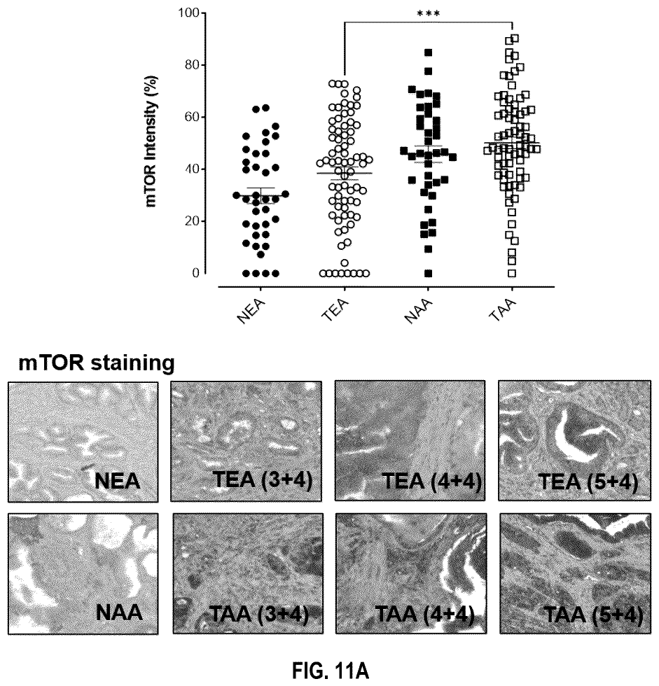

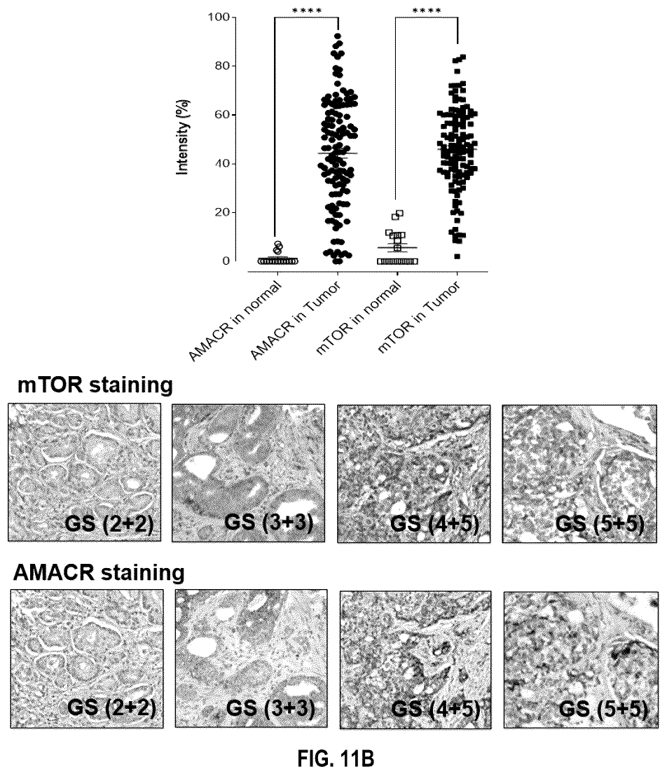

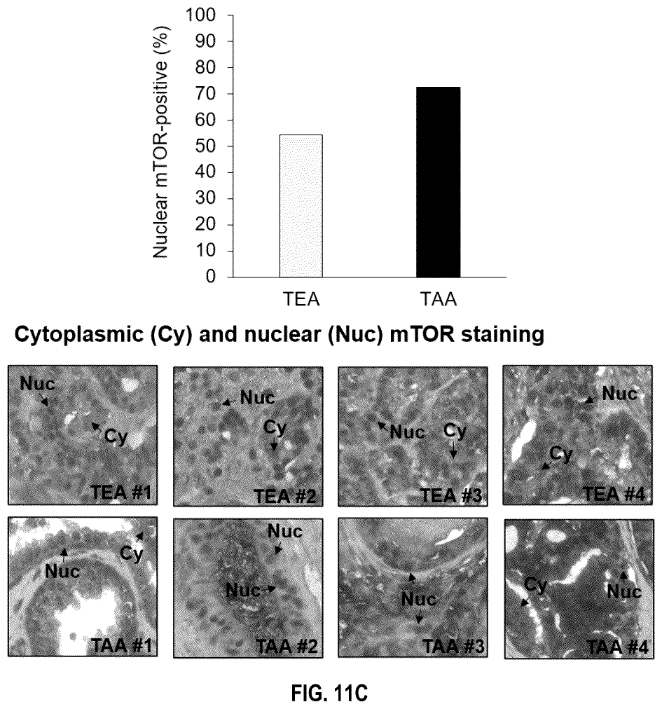

In Example 1, mRNA profiling, RT-qPCR, and Western blot results demonstrated that mTOR, targeted and inhibited by miR-99b-5p, is upregulated in African American prostate cancer vs. European American prostate cancer. To further evaluate whether mTOR could serve as a potential diagnostic and/or prognostic biomarker for African American prostate cancer (or prostate cancer in general), IHC assays were performed to examine the mTOR expression levels in prostate cancer specimens derived from two independent cohorts of prostate cancer patients. Firstly, a formalin-fixed paraffin-embedded (FFPE) tissue microarray (TMA), containing cancerous specimens and adjacent normal tissues from 40-50 European American and 40-50 African American prostate cancer patients, and normal prostate tissues from 3 European American and 3 African American healthy individuals, was used to evaluate whether mTOR is differentially expressed between European American prostate cancer and African American prostate cancer. The IHC staining results have confirmed that African American prostate cancer exhibited higher mTOR expression when compared to European American prostate cancer. Quantification of mTOR intensities demonstrated that mTOR protein levels in tumorous African American prostate cancer (TAA) specimens were significantly higher than tumorous European American prostate cancer (TEA) specimens (FIG. 11A, left panel). By comparing African American prostate cancer and European American prostate cancer samples with comparable Gleason scores (GS), significantly higher mTOR intensities were also observed in African American prostate cancer vs. European American prostate cancer specimens (FIG. 11A, right panel). Notably, significantly higher mTOR expression was detected even in noncancerous NAA samples vs. NEA samples (FIG. 11A, left and right panels), suggesting that mTOR may be a predisposing risk factor in African Americans (who have higher chance to develop aggressive types of prostate cancer).

Secondly, two TMAs containing 16 normal prostate tissues, 7 hyperplasia specimens, and 169 prostate cancer specimens (from US Biomax Inc.) were used to examine the mTOR and α-methylacyl CoA racemase (AMACR) expression levels, respectively. Several reports indicated that AMACR has emerged as a prostate cancer biomarker, and AMACR expression level has also been associated with prostate cancer progression and prognosis. As shown in FIG. 11B, AMACR and mTOR expression levels were significantly higher in prostate cancer specimens than in normal tissues. Overall, comparable expression levels of AMACR and mTOR were observed in prostate cancer samples (FIG. 11B, left panel). Moreover, mTOR intensities seems to be positively correlated to the AMACR levels in the prostate cancer samples derived from the same existing patients (FIG. 11B, right panel). These results suggest that mTOR, with similar AMACR expression profile in prostate cancer specimens, may potentially serve as a potential biomarker for diagnosis and/or prognosis in prostate cancer. The IHC assays have further revealed that mTOR was expressed in both cytoplasmic and nuclear fractions of the prostate cancer samples (FIG. 11C, left and right panels). Notably, a higher percentage of African American prostate cancer specimens (72.6%, 53 out of 73 African American prostate cancer specimens expressed nuclear mTOR) demonstrated nuclear mTOR signals, when compared to the European American prostate cancer specimens (54.3%, 38 out of 70 European American prostate cancer samples expressed nuclear mTOR) (FIG. 11C, left panel). Considering the aggressive nature of African American prostate cancer (i.e., higher recurrence and mortality rates), it raised an interesting question as to whether the high nuclear mTOR level (a more oncogenic form of mTOR) functionally contributes to the African American prostate cancer aggressiveness.

Differential Subcellular Distributions of mTOR and pmTOR in African American Prostate Cancer and European American Prostate Cancer

To further explore the potential functional roles of cytoplasmic and nuclear mTOR in African American prostate cancer, immunofluorescence assays were conducted in three European American prostate cancer (22Rv1, LNCaP, and PC-3) and two African American prostate cancer (RC77 T/E, and MDA PCa 2b) cell lines. 22Rv1 represents an androgen-independent European American prostate cancer cell line model, while LNCaP and PC-3 are metastatic European American prostate cancer cell lines derived from lymph node and bone metastasis. RC77 T/E was used as a primary African American prostate cancer cell model, while MDA PCa 2b was used as a metastatic African American prostate cancer cell line derived from bone metastasis. The immunofluorescence assays have revealed higher levels of cytoplasmic mTOR vs. nuclear mTOR in 22Rv1, LNCaP, and PC-3 cells, whereas nearly equal distribution of cytoplasmic and nuclear mTOR were observed in RC77 T/E and MDA PCa 2b (green fluorescent mTOR signals and DAPI signals in FIG. 12A). Specifically, ˜80% of African American prostate cancer (RC77 T/E and MDA PCa 2b) cells expressed nuclear mTOR, but only 30-45% of European American prostate cancer (22Rv1, LNCaP, and PC-3) cells expressed nuclear mTOR (FIG. 12B). In addition, significantly higher pmTOR (the active form of mTOR) levels were shown in both cytoplasmic and nuclear fractions of African American prostate cancer cell lines than European American prostate cancer cell lines (FIG. 12A, FIG. 12C). Notably, metastatic African American prostate cancer (MDA PCa 2b) cells exhibited the highest ratio of nuclear pmTOR/total mTOR (˜80%, calculated based on the number of nuclear pmTOR-positive cells/the number of mTOR-positive cells×100%) among all prostate cancer cell lines (red fluorescence, DAPI and merged signals in FIG. 12A, and quantification data in FIG. 12C). The differential distributions of cytoplasmic/nuclear mTOR and pmTOR between African American prostate cancer and European American prostate cancer suggest that more oncogenic events (resulted from higher level of nuclear mTOR-mediated transcriptional activation of downstream/metabolic genes in chromatin) may occur in African American prostate cancer vs. European American prostate cancer. This raises an interesting question whether and how the elevated nuclear mTOR and pmTOR protein levels contributes to the more aggressive phenotypes of African American prostate cancer.

Transfection of miR-99b-5p Inhibits the Expression Levels and Nuclear Translocation of Total mTOR and Nuclear pmTOR in Prostate Cancer Cells

To assess the hypothesis that the African American prostate cancer aggressiveness may be due to the overall upregulation of nuclear mTOR and pmTOR, nonsense/scrambled RNA (NS) or miR-99b-5p mimic was transfected to the European American prostate cancer and African American prostate cancer cell lines then followed by examining the subcellular distributions/expression levels of mTOR and pmTOR by using immunofluorescence assays and Western blot analysis. A generalized reduction in total mTOR (green fluorescence) and pmTOR (red fluorescence) signals were observed across all European American and African American prostate cancer cell lines upon miR-99b-5p transfections (FIG. 13A). Intriguingly, higher ratio of cytoplasmic pmTOR and lower ratio of nuclear pmTOR signals were observed in prostate cancer cells transfected with miR-99b-5p mimic vs. NS control. This phenomenon is particularly evident in the African American prostate cancer cell lines RC77 T/E and MDA PCa 2b, where the high nuclear pmTOR/mTOR signals in NS transfected cells but exclusively cytoplasmic pmTOR/mTOR signals in miR-99b-5p transfected cells were observed (merged images, RC77 T/E and MDA PCa 2b transfected with NS vs. miR-99b-5p mimic, FIG. 13A). Quantification of both cytoplasmic and nuclear pmTOR signals further confirmed that the cytoplasmic pmTOR signals were increased and the nuclear pmTOR signals were reduced significantly upon miR-99b-5p overexpression in all European American and African American prostate cancer cell lines (FIG. 13B). Western blot assays were used to further verify the protein dynamics/distribution of mTOR and pmTOR in the cytoplasmic and nuclear fractions of European American and African American prostate cancer cells. The European American and African American prostate cancer cell lines were transfected with NS or miR-99b-5p mimic for 48 h, the cytoplasmic and nuclear proteins were prepared and subjected to the Western blot analysis. Consistent with the immunofluorescence results, the Western blot assays have again demonstrated a moderate to significant reduction in mTOR and pmTOR in nuclear fractions from all European American and African American prostate cancer cell lines transfected with miR-99b-5p mimic (FIG. 13C, FIG. 14). It is particularly evident from the significant decrease in nuclear mTOR and pmTOR in the metastatic African American prostate cancer cell line MDA PCa 2b (FIG. 13C, FIG. 14). In contrast, miR-99b-5p transfection caused a slight to no reduction in cytoplasmic mTOR in all the prostate cancer cell lines (FIG. 13C, FIG. 14). Taken together, these results strongly suggest that miR-99b-5p overexpression inhibits mTOR expression and may also blocks the translocation of mTOR (as well as pmTOR) from cytoplasm to nucleus in prostate cancer.

Upregulation of mTOR and Downregulation of miR-99b-5p in Colon, Breast and Lung Cancer Specimens and Cell Lines

To further evaluate whether the reciprocal mTOR/miR-99b-5p pairing can also serve as a potential biomarker for solid tumors in general, IHC, Western blot, and RT-qPCR assays were performed to examine the expression levels of the mTOR protein and miR-99b-5p in colon, breast, and lung cancers. The IHC assay on a TMA containing multiple cancer specimens has shown that the mTOR protein is overexpressed in colon, breast, and lung cancer patient specimens (FIG. 15A). Statistically, the colon and lung cancer specimens expressed comparable mTOR levels when compared to prostate cancer samples in the TMA, while breast cancer specimens expressed lower levels of mTOR protein than the prostate cancer specimens (FIG. 15A). Furthermore, IHC staining results have revealed that increased mTOR levels were detected in high-grade/advanced colon, breast, and lung cancer specimens. For instance, the highest mTOR intensities were detected in grade 3 cancers, while moderate and low mTOR intensities were detected in grade 2 and 1 patient specimens, respectively (FIG. 15B). The positive correlation between mTOR staining intensities and tumor grades suggests that the mTOR expression profile may be a potential index/biomarker for evaluating cancer aggressiveness. Interestingly, the survival curves generated from RNA-sequencing (RNA-seq) database of the Cancer Genome Atlas (TCGA) also demonstrated that a higher mTOR expression level is correlated with an overall poorer survival in colon, breast, lung, and pancreatic cancer patients (FIG. 16). These results suggest that the mTOR expression profile may potentially serve as a precision prognostic biomarker (i.e., correlated to cancer grades/aggressiveness, clinical outcomes, etc.) for prostate cancer and other solid tumors.

To further validate mTOR and miR-99b-5p expression levels in the in vitro cancer cell line models, Western blot analysis and RT-qPCR assays were conducted in a panel of colon, breast, lung, and prostate cancer cell lines. First, the Western blot analysis has shown that mTOR is upregulated in colon cancer (HT-29 and SW620) vs. normal colon (FHC) cells, in breast cancer (MDA MB 231, MCF-7) vs. normal breast (HMEC) cells, in lung cancer (A549 and H1299) (FIG. 15C). Note that all the cancer cell lines expressed similar mTOR levels when compared to the mTOR-expressing prostate cancer cell lines (PC-3 and MDa PCa 2b). The only exception is that normal lung cell line BEAS-2B also expresses mTOR with comparable level as other cancer cells (FIG. 15C).

Next, the expression levels of miR-99b-5p (a negative regulator of mTOR) were examined in the same panel of the cancerous and normal cell lines used in Western blot analysis. RT-qPCR results have confirmed that miR-99b-5p was significantly downregulated in colon cancer (HT-29 and SW620) vs. normal colon (FHC) cells, in breast cancer (MDA MB 231, MCF-7) vs. normal breast (HMEC) cells, and in lung cancer (H1299) vs. normal lung (BEAS-2B) cells (FIG. 15D). The negative correlation of mTOR and miR-99b-5p expression levels in colon, breast, and lung cancer cells indicated that the regulatory effects of the reciprocal pairing miR-99b-5p/mTOR (down/up) may play a crucial role in advanced colon, breast, and lung cancers, similar to what was observed in the African American prostate cancer.

Overexpression of miR-99b-5p Reduces Expression of mTOR and pmTOR, Inhibits the Nuclear Translocation of pmTOR and Induces Cell Apoptosis in Various Cancer Cells

Various cancer cell lines (HT-29, SW620, MDA MB 231, MCF-7, A549, and H1299) were used as solid tumor cell models to investigate the functional impacts of miR-99b-5p overexpression in mTOR expression and subcellular distribution. Similar to the effects of miR-99b-5p in prostate cancer cells, all the six cancer cell lines showed a generalized reduction in mTOR (green fluorescence) and pmTOR (red fluorescence) levels in miR-99b-5p transfected vs. NS transfected cells (FIG. 17A). Further analyzing the subcellular distribution of mTOR and pmTOR has revealed elevated cytoplasmic mTOR/nuclear mTOR ratios (as well as cytoplasmic pmTOR/nuclear pmTOR ratios) in miR-99b-5p mimic vs. NS transfected cancer cells (FIG. 17A). Specifically, higher levels of nuclear mTOR and/or nuclear pmTOR signals were observed in the NS-transfected cells (green, red fluorescence, and yellow merge in nuclei, FIG. 17A). In contrast, the mTOR and pmTOR were mostly expressed in the cytoplasm of the miR-99b-5p mimic transfected cells (green and red in cytoplasm, while slight to no green/red colocalization in nuclei merge, miR-99b-5p panel in FIG. 17A). These results suggest that miR-99b-5p targets/inhibits mTOR in cancer cells, subsequently blocking mTOR translocation from cytoplasm to nucleus. Furthermore, TUNEL assays were conducted to assess whether the miR-99b-5p overexpression can induce the cell apoptosis. As shown in FIG. 17B, TUNEL assay results have demonstrated enhanced DNA breakages (i.e., increased red fluorescent signals in nuclei) in the miR-99b-5p mimic vs. NS transfected cancer cells, implicating that miR-99b-5p initiates/promoting cell apoptosis in the cancer cells.

Transfection of miR-99b-5p Mimic Inhibits AR-Mediated mTOR Translocation and Promotes the Docetaxel-Induced Cytotoxicity in Various Cancers

To explore the molecular mechanism of miR-99b-5p-mediated mTOR expression and cytoplasmic/nuclear mTOR dynamics, Western blot analyses were performed after the cancer cells were transfected with NS or the miR-99b-5p mimic in the absence or presence of docetaxel. First, Western blot analysis has again confirmed that miR-99b-5p negatively regulates mTOR protein expression in these cancer cells, evident from a generalized reduction in total mTOR (combined protein levels of cytoplasmic and nuclear mTOR) and a significant decrease in nuclear mTOR (and nuclear pmTOR) expression in miR-99b-5p vs. NS transfected cells

(FIG. 18A, FIG. 19). In contrast, slight to no decrease in cytoplasmic mTOR was observed in cancer cells upon miR-99b-5p transfection (FIG. 18A, FIG. 19). These results, again, suggest that miR-99b-5p targets/inhibits mTOR and mediates (directly or indirectly) mTOR translocation to the nucleus.

Androgen receptor (AR) plays a central role for prostate cancer pathogenesis. Previous studies have revealed that AR activation mediates the mTOR signaling, and the formation of AR-mTOR complex is required for the translocation of mTOR from cytoplasm to nucleus. However, it remains unknown how miR-99b-5p participates at the AR-mTOR axis and regulates the mTOR translocation to nucleus. To explore the possible molecular mechanism underlying the miR-99b-5p-mediated cytoplasmic/nuclear mTOR dynamics and the downstream effects on cell survival/apoptosis, Western blot analyses were performed to examine the protein levels of AR in total, cytoplasmic, and nuclear protein fractions. Breast cancer (MCF-7) and prostate cancer (22Rv1 and MDA PCa 2b) cell lines that express considerable levels of AR were used as in vitro cell line models to study the miR-99b-5p involvement in AR-mTOR axis. Similar to the miR-99b-5p effect on mTOR expression/location, the AR expression levels were significantly reduced in miR-99b-5p mimic vs. NS transfected cancer cells (total lysates, FIG. 18B). Moreover, nuclear AR protein levels were significantly decreased in MCF-7, 22Rv1, and MDA PCa 2b cells. In contrast, miR-99b-5p overexpression caused no change of cytoplasmic AR in MCF-7 cells, and slight to moderate decrease in cytoplasmic AR in 22Rv1 and MDA PCa 2b cells (FIG. 18B). Taken together, the data suggest that miR-99b-5p negatively regulates mTOR and AR, subsequently inhibiting the translocation of AR/mTOR complex from cytoplasm to nucleus.

Next, whether the miR-99b-5p can target/inhibit the AR/mTOR axis and promote cell apoptosis and/or enhance the docetaxel-induced cytotoxicity in colon, breast, and lung cancers was further investigated. The caspase 3/7 activity-based apoptosis assays have revealed that miR-99b-5p as a single agent was not sufficient to initiate significant apoptosis in the tested cancer cells. However, miR-99b-5p overexpression induced moderate to significant cell apoptosis in the presence of chemotherapeutic agent docetaxel in all cancer cell lines, except MDA MB 231 (FIG. 18C). These results, similar to the previous observation in prostate cancer, again suggest that miR-99b-5p targets/inhibits mTOR signaling and consequently sensitizes the docetaxel-induced cytotoxicity in the advanced cancer cells.

### Materials and Methods

### Cell Culture

The human cell lines used in the study included: 22Rv1, LNCaP, PC-3, RC77 T/E and MDA PCa 2b (prostate cancer cell lines), MDA MB 231 and MCF-7 (breast cancer cell lines), HT-29 and SW620 (colon cancer cell lines), A549 and H1299 (lung cancer cell lines). RWPE-1, HMEC, FHC, and BEAS-2B were used as normal control cell lines for prostate, breast, colon, and lung, respectively. Note that 22Rv1 is a castration-resistant European American prostate cancer cell line, while LNCaP and PC-3 were derived from lymph node and bone metastasis of European American prostate cancer patients, respectively. RC77 T/E was derived from a primary African American prostate cancer patient, and MDA PCa 2b was derived from bone metastasis of an African American prostate cancer patient. The cell lines described were used as in vitro cell models to evaluate the functional roles of miR-99b-5p/MTOR pairing in the European American prostate cancer, African American prostate cancer and solid tumors in general. The cells were grown in specific cell culture media described as follows: 22Rv1, LNCaP, MCF-7 and H1299 were cultured in RPMI-1640 with 10% fetal bovine serum (FBS), PC-3, A549 and FHC were cultured in DMEM with 10% FBS, RC77 T/E was cultured in Keratinocyte SFM with human recombinant epidermal growth factor (EGF) and bovine pituitary extract (BPE), MDA PCa 2b were cultured in BRFF-HPC1 with 20% FBS, HT-29 was cultured in McCory's with 10% FBS, SW620 and MDA MB 231 were cultured in L-15 with 10% FBS, HMEC was cultured in mammary epithelial cell basal medium with supplements, and BEAS-2B was cultured in BEBM base medium with BEBM supplement kit. Cells were maintained at 37° C. in a 5% CO₂ incubator.

Transfection of miR-99b-5p Mimic and Nonsense/Scrambled RNA in Cell Line Models

The 22Rv1, LNCaP, PC-3, MDA PCa 2b, MCF7, H1299, A549, HT-29, SW620, MDA MB-231, HMEC and BEAS-2B cells were seeded at a density of 3×10⁵ cells/well in 6-well plates. RC77 T/E cells were seeded at a density of 5×10⁵ cells/well in 6-well plates. The cells were grown for 24 h and then transfected with nonsense/scrambled RNA (NS) and miR-99b-5p mimic (Ambion) using DharmaFECT4 transfection reagent (Dharmacon). After 24 h, fresh media were applied to replace the transfection reagent-containing media, then the cells were incubated for an additional 24 h.

### Tissue Microarrays (TMAs)

Three types of TMAs were used in this study. First, a TMA containing European American and African American prostate cancer samples, adjacent normal prostate tissues from 40-50 European American and 40-50 African American prostate cancer patients, as well as normal prostate tissues from 3 healthy European American and 3 African American healthy individuals was used to evaluate the mTOR expression levels using IHC assay. This type of TMA was designed and prepared by the Department of Pathology at University of Maryland Baltimore (UMB). Second, TMAs containing normal prostate tissue and prostate cancer specimens were purchased from US Biomax Inc. (catalog #PR208a). The prostate cancer TMAs were used to examine the mTOR and AMACR expression levels in the prostate cancer patient samples and adjacent normal tissues included on the TMAs. Third, a TMA containing tumor samples derived from patients diagnosed with breast cancer, colon cancer, and lung cancer (US Biomax catalog #BC000119b) was used for examining the expression levels of mTOR.

### Immunohistochemistry (IHC) Assays

Slides containing serial sections were deparaffinized in xylene (2×5 min), followed by immersion in xylene/alcohol solution (xylene:ethanol=1:1) and slowly rehydrated through graded alcohols (100%, 95%, 70% and 50% of alcohol, respectively) to distilled water. EnVision FLEX target retrieval solution from Agilent technologies was purchased and diluted according to the manufacturer's protocol. Antigen retrieval was performed in microwave for 25 min (with full power for 5 min, and 20% power for an additional 20 min), followed by cooling down sections for 30 min at room temperature. The sections were then rinsed with running cold water for 10 min. Peroxidase block was added dropwise and incubated for 30 min at room temperature. The slides were then washed with 1×PBS twice for 5 min followed by adding blocking buffer (2.5% BSA in 1×PBS) and incubating for 30 min at room temperature. After discarding blocking buffer, tissue sections were incubated with the primary antibody (1:100-1:200 dilutions in 2.5% BSA/1×PBS) at 4° C. overnight. On the following day, sections were washed with 1×PBS twice for 5 min and incubated with HRP-conjugated secondary antibody (Dako) for 30 min, and HRP was detected by diaminobenzidine (DAB; Dako). Tissue sections were counterstained with Mayer's hematoxylin (Sigma) for 1 min at room temperature, incubated in 0.037M ammonia for 1 min, washed with distilled water for 5 min, and mounted with glycergel mounting medium (Dako).

IHC images were captured using Pannormic Midi Digital Scanner (3DHISTECH Ltd.) and viewed using Case Viewer program developed by 3DHISTECH. The analysis and quantification of IHC images were performed using ImageJ software (NIH). Each individual tissue sample on the TMAs was selected and converted to an 8-bit image, followed by measuring threshold. Two separate values were calculated. First, the region of total epithelial area (ROT) was defined and measured using ImageJ. Second, the total area with actual DAB staining signals in the defined epithelial structures was measured (region of interest, ROI). The relative mTOR staining intensities were determined based on the calculation of ROI/ROT×100%. The statistical analysis was performed using ANOVA with Tukey's post-hoc test for the multiple comparisons. The mTOR and AMACR antibodies were purchased from Cell Signaling Technology and Agilent Technologies, respectively.

RT-qPCR Validation of miR-99b-5p

To measure the expression level of miR-99b-5p in various cell lines, RT-qPCR assay was performed. Total RNA was isolated using the miRNeasy Mini kit (Qiagen) from each cell line. To quantify total RNA of samples, Nano Vuc Plus spectrophotometer (GE Healthcare) was used. Reverse transcription was performed using miRCURY LNA RT kit (Qiagen), where 1 μg of total RNA was used as a template. Once cDNA was synthesized, quantitative PCR (qPCR) assay was performed using miRCURY LNA SYBR Green PCR kit (Qiagen). The miRNA primers designed for amplification of hsa-miR-99b-5p and hsa-miR-103a-3p were based on the specific miRCURY miRNA Assays purchased from Qiagen. The qPCR reaction program was set as follows: pre-denaturation for 5 min at 95° C., followed by 40 standard cycles of: denaturation at 95° C. for 15 s, annealing at 55° C. for 30 s, and extension at 70° C. for 30 s. To determine the miRNA expression levels, qPCR reactions were performed in duplicates or triplicates from 3 independent RNA samples, using endogenous miR-103a-3p for data normalization. Normalized gene expression levels were determined using the 2^{-ΔCT} method.

### Western Blot Analysis

The total proteins were extracted using M-PER extraction reagent with protease and phosphatase inhibitor cocktail (Thermo Fisher Scientific) according to manufacturer's protocol. Cytoplasm and nuclear proteins were extracted using Subcellular Protein Fractionation kit (Thermo Fisher Scientific) according to the manufacturer's protocol. Equal amounts of proteins were used based on the quantification using BCA assay kit (Thermo Fisher Scientific), and the samples were separated by electrophoresis using NuPAGE 4-12% or 8% Bis-Tris gels (Invitrogen). The gels were transferred to PVDF membranes (Bio-Rad) then the PVDF membranes were incubated with SuperBlock blocking buffer (Thermo Fisher Scientific). After 1 h, the PVDF membranes were incubated with primary antibodies overnight at 4° C., washed 3 times with 1×TBST, and then incubated with secondary antibody for 1 h at room temperature, then washed 5 times with 1×TBST. The results were analyzed with SuperSignal ECL substrates (Thermo Fisher Scientific) and a ChemiDoc XRS system (Bio-Rad). The primary and secondary antibodies used in the study were mTOR, pmTOR, AR, GAPDH, Lamin B1, β-actin, and anti-rabbit IgG-HRP antibodies from Cell Signaling Technology.

### Immunofluorescence Staining

In this process, 4×10⁴ cells were seeded on cover slip and allowed to adhere for 24 h in 5% CO₂ incubator at 37° C. All the cells were subjected to immunofluorescence assays 48 h following transfection. Briefly, cells were washed with 1×PBS, fixed in 4% paraformaldehyde, and permeabilized with 0.1% Triton X-100. Cells were then blocked for 1 h with 2% BSA in 1×PBS. Primary antibodies against mTOR (Cell Signaling Technology) and pmTOR (Santa Cruz Biotechnology) were applied to the fixed/permeabilized cells for incubation overnight at 4° C. The cells were washed twice with 1×PBS, and followed by incubating with Alexa-Fluor-488-conjugated anti-rabbit and Alexa-Fluor-594-conjugated anti-mouse antibodies, respectively (catalog #A32731 and #A32744, from Invitrogen) for 1 h at room temperature. Thereafter, cells were washed twice with 1×PBS for 5 min and the nuclei were visualized by staining with DAPI from Invitrogen (catalog #P36981). All labeled and/or prepared cells mounted on glass slides and were visualized by fluorescence microscopy (Olympus). Cell images were captured from 3-4 random areas at 20× magnification by using CellSens V1.18 software (Olympus).

TdT-Mediated dUTP-Biotin End-Labeling (TUNEL) Assays

TUNEL assay was carried out according to the manufacturer's protocol (Click-iT™ Plus In-situ Apoptosis Detection with Alexa Fluor Dyes, Thermo Fisher Scientific). Precisely, 4×10⁴ cells (HT-29, SW620, MDA MB 231, MCF-7, A549, and H1299) were seeded on coverslips and allowed to adhere for 24 h in 5% CO₂ incubator at 37° C. These cells were fixed in 4% paraformaldehyde in 1×PBS after exposure of miR-99b-5p mimic or negative control mimic for 48 h, then washed with PBS three times followed by the incubation with 0.25% Triton X-100 in 1×PBS and ultimately the cells were incubated with differential steps of TUNEL reaction mixture as per the manufacturer's protocol. Lastly, the processed cells were counterstained with DAPI for 5 min, at room temperature in the dark. All labeled and/or prepared cells were mounted on glass slides and were visualized by Olympus BX3 fluorescence microscope (Olympus). Cell images were captured from 3-4 random areas at 10× magnification by using CellSens V1.18 software (Olympus).

### Caspase 3/7 Activity-Based Apoptosis Assay

HT-29, SW620, MDA MB 231, MCF7, A549, and H1299 were seeded at a density of 3×10⁴ cells/well in 96-well plates. The cells were grown overnight and then followed by transfections. After 24 h, fresh media were applied to replace the transfection reagent-containing media with 11 mM of docetaxel or vehicle, then the cells were incubated for an additional 24 h. The Apo-ONE Caspase-3/7 Assay Kit (Promega Corporation) was used to measure apoptosis according to the protocol described by the manufacturer. Then, 100 μL of homogeneous Caspase-3/7 reagent was added to each well and the plate incubated at room temperature for 30 min to 2 h. Fluorescence was detected for measuring (at wavelengths of 499/521 nm excitation/emission) the apoptosis state (Caspase 3/7 activity) using Biotek Synergy HT Microplate Reader (BioTek).

### DISCUSSION

In the current study, whether miR-99b-5p/MTOR can serve as a precision diagnostic and/or prognostic biomarker in prostate cancer and other solid tumors including colon, breast and lung cancers was investigated. The IHC, Western blot, and RT-qPCR results have confirmed an overall upregulation of mTOR and downregulation of miR-99b-5p in independent cohorts of patient samples and a panel of cell lines derived from prostate, colon, breast and lung cancers. In vitro functional assays further demonstrated that miR-99b-5p targets AR-mTOR signaling, resulting in inhibiting AR and mTOR expression, blocking nuclear translocation of mTOR, enhancing cell apoptosis, and sensitizing docetaxel-induced cytotoxicity in prostate cancer cells. Similar functional effects of miR-99b-5p on AR/mTOR inhibition, mTOR localization, and apoptosis induction were also shown in colon, breast, and lung cancer cell models.

Previous studies have shown that the tumor suppressive miR-99b-5p is downregulated in various cancers including prostate cancer. In contrast, mTOR is upregulated in a variety of cancer types. Example 1 further suggested a potential clinical application of utilizing reciprocal miR-99b-5p/mTOR (down/up) pairing as a diagnostic/prognostic biomarker in African American prostate cancer. In this study, it was further confirmed that mTOR is upregulated in African American prostate cancer vs. European American prostate cancer and prostate cancer vs. normal (FIG. 11). Furthermore, an inverse correlation between miR-99b-5p and mTOR expression levels was confirmed in other solid tumors including colon, breast, and lung cancers (FIG. 15). Although data analysis from TCGA RNA-seq data has demonstrated an overall lower survival rate in cancers expressing high-level vs. low-level MTOR transcript, the differences were not statistically significant (FIG. 16). Further survival data analysis by extracting RNA-seq data from patients expressing high-level MTOR but low-level miR-99b-5p may further validate whether reciprocal miR-99b-5p/MTOR (down/up) pairing is a better prognostic biomarker than mTOR alone.

Another interesting observation is that African American prostate cancer exhibits a higher level of nuclear mTOR than European American prostate cancer. This raises a challenging question of whether the elevated nuclear mTOR expression contributes to the more aggressive properties observed in African American prostate cancer. Emerging studies have indicated the unique functions of nuclear mTOR in cancers including prostate cancer. Although the mTOR protein is mainly localized in cytoplasm, nuclear mTOR and its oncogenic impacts have been implicated in several tumors, including gastric cancer, endometrial cancer, thyroid cancer, prostate cancer and multiple myeloma. For instance, higher nuclear mTOR expression has been associated with poor prognosis in endometrial, thyroid, and prostate cancers.

Overexpression of miR-99b-5p has been reported to negatively regulate the protein expression of mTOR, AR and prostate specific antigen (PSA), consequently inhibiting the cell proliferation, migration, inducing autophagy, promoting apoptosis, and sensitizing the docetaxel-induced cytotoxicity in prostate cancer. AR has also been implicated in the coordination of protein complex diversity and subcellular localization of mTOR, including regulating the translocation of mTOR to the nucleus and the mTOR-mediated gene networks. It is reported that AR activation leads to the upregulation of mTOR signaling, and enhances mTOR translocation from cytoplasm to nucleus. ChIP-seq and ChIP-qPCR results further revealed that AR and mTOR colocalize at the same genomic loci, and the mTOR-chromatin binding is driven in an AR-dependent manner in prostate cancer cells. Mechanistically, the nuclear mTOR/AR signaling axis mediates the metabolic reprogramming in prostate cancer. Consistent with the previous studies, the data have demonstrated that miR-99b-5p negatively regulates mTOR and AR expression, initiates cell apoptosis, and promotes the docetaxel-induced cytotoxicity in prostate cancer and other solid tumor cells. Moreover, the immunofluorescence and Western blot analysis further revealed that miR-99b-5p inhibits nuclear translocation of mTOR. This is particularly evident form the significant reduction in nuclear mTOR, pmTOR and AR, but slight/moderate to no reduction in cytoplasmic mTOR and AR in prostate cancer and other cancer cells upon miR-99b-5p mimic transfection (FIG. 13, FIG. 17, FIG. 18). Here, a molecular model that miR-99b-5p negatively regulates the expression level of AR and mTOR, thereby decreasing the overall level of AR/mTOR complex for nuclear translocation was proposed. In fact, miR-99b-5p also targets/inhibits SMARCD1, a cofactor of active AR, which may also involve in the miR-99b-5p-mediated nuclear translocation of mTOR. This is the first report to propose a functional mechanism of miR-99b-5p/AR/mTOR signaling axis in regulating prostate cancer aggressiveness and progression. Previous study has also demonstrated that IGF1R (encoding insulin-like growth factor 1 receptor, IGF-1R), an upstream gene of AKT/mTOR signaling, is a direct target of miR-99b-5p in gastric cancer. Although no differential IGF1R expression in African American vs. European American prostate cancer was identified from the mRNA profiling data, the downregulation of miR-99b-5p may additionally stimulate the IGF-1F-mediated AKT/mTOR signaling in prostate cancer progression, regardless of the ethnicities.

In conclusion, the study provides a molecular insight into how miR-99b-5p/AR/mTOR axis regulates the prostate cancer aggressiveness and progression. Instead of considering total mTOR as an oncogenic indicator, the results suggest that reciprocal miR-99b-5p/“nuclear” mTOR pairing (down/up) pairing may serve as a potential precision diagnostic and prognostic biomarker for prostate cancer. Further developing a dual staining protocol for miR-99b-5p and nuclear mTOR (or nuclear pmTOR) using RNAScope/IHC technology may facilitate the development of a precision diagnostic/prognostic biomarker for aggressive prostate cancer. Lastly, a deeper understanding of the molecular mechanism underlying miR-99b-5p-mediated AR/mTOR signaling axis and AR/mTOR cytoplasmic/nuclear dynamics may pave a new path for developing novel therapeutics to treat the aggressive prostate cancer.

### REFERENCES

Abramovic, I.; Ulamec, M.; Bojanac, A. K.; Bulic-Jakus, F.; Jezek, D.; Sinic, N. miRNA in prostate cancer: Challenges toward translation. Epigenomics 2020, 12, 543-558.
Aftab, D. T.; McDonald, D. M. MET and VEGF: Synergistic targets in castration-resistant prostate cancer. Clin. Transl. Oncol. 2011, 13, 703-709.
Andres-Leon, E.; Cases, I.; Alonso, S.; Rojas, A. M. Novel miRNA-mRNA interactions conserved in essential cancer pathways. Sci. Rep. 2017, 7, 46101.
Audet-Walsh, E.; Dufour, C. R.; Ycc, T.; Zouanat, F. Z.; Yan, M.; Kalloghlian, G.; Vernier, M.; Caron, M.; Bourque, G.; Scarlata, E.; et al. Nuclear mTOR acts as a transcriptional integrator of the androgen signaling pathway in prostate cancer. Genes Dev. 2017, 37, 1228-1242.
Audet-Walsh, E.; Vernier, M.; Ycc, T.; Laflamme, C.; Li, S.; Chen, Y.; Giguere, V. SREBF1 Activity Is Regulated by an AR/mTOR Nuclear Axis in Prostate Cancer. Mol. Cancer Res. 2018, 16, 1396-1405.
Awasthi, S.; Berglund, A.; Abraham-Miranda, J.; Rounbehler, R. J.; Kensler, K.; Serna, A.; Vidal, A.; You, S.; Freeman, M. R.; Davicioni, E.; et al., Comparative Genomics Reveals Distinct Immune-oncologic Pathways in African American Men with Prostate Cancer. Clin. Cancer. Res. 2021, 27, 320-329.
Balacescu, O.; Dumitrescu, R. G.; Marian, C. MicroRNAs Role in Prostate Cancer. Methods Mol. Biol. 2018, 1856, 103-117.
Conti, D. V.; Darst, B. V.; Moss, L. C.; Saunders, E. J.; Sheng, X.; Chou, A.; Schumacher,
F. R.; Olama, A. A. A.; Benlloch, S.; Dadacv, T.; et al. Trans-ancestry genome-wide association meta-analysis of prostate cancer identifies new susceptibility loci and informs genetic risk prediction. Nat. Genet. 2021, 53, 65-75.
de Nobrega, M.; Ciliao, H. L.; de Souza, M. F.; de Souza, M. R.; Serpeloni, J. M.; Fuganti, P. E.; de Colus, I. M. S. Association of polymorphisms of PTEN, AKT1, PI3K, AR, and AMACR genes in patients with prostate cancer. Genet. Mol. Biol. 2020, 43, c20180329.
Evans, A. J.; Alpha-methylacyl CoA racemase (P504S): Overview and potential uses in diagnostic pathology as applied to prostate needle biopsies. J. Clin. Pathol. 2003, 56, 892-897.
Fabris, L.; Ceder, Y.; Chinnaiyan, A. M.; Jenster, G. W.; Sorensen, K. D.; Tomlins, S.; Visakorpi, T.; Calin, G. A. The Potential of MicroRNAs as Prostate Cancer Biomarkers. Eur. Urol. 2016, 70, 312-322.
Fu, P.; Bu, C.; Cui, B.; Li, N.; Wu, J. Screening of differentially expressed genes and identification of AMACR as a prognostic marker in prostate cancer. Andrologia 2021, 53, c14067.
Guglielmelli, T.; Giugliano, E.; Brunetto, V.; Rapa, I.; Cappia, S.; Giorcelli, J.; Rrodhe, S.; Papotti, M.; Saglio, G. mTOR pathway activation in multiple myeloma cell lines and primary tumour cells: Pomalidomide enhances cytoplasmic-nuclear shuttling of mTOR protein. Oncoscience 2015, 2, 382-394.
Gujrati, H.; Ha, S.; Mohamed, A.; Wang, B. D. MicroRNA-mRNA Regulatory Network Mediates Activation of mTOR and VEGF Signaling in African American Prostate Cancer. Int. J. Mol. Sci. 2022, 23, 2926.
Huang, T. F.; Wang, S. W.; Lai, Y. W.; Liu, S. C.; Chen, Y. J.; Hsuch, T. Y.; Lin, C. C.; Lin, C. H.; Chung, C. H. 4-Acetylantroquinonol B Suppresses Prostate Cancer Growth and Angiogenesis via a VEGF/PI3K/ERK/mTOR-Dependent Signaling Pathway in Subcutaneous Xenograft and In Vivo Angiogenesis Models. Int. J. Mol. Sci. 2022, 23, 1446.
Jiang, B. H.; Liu, L. Z. Role of mTOR in anticancer drug resistance: Perspectives for improved drug treatment. Drug Resist. Updates 2008, 11, 63-76.
Jiang, N.; Zhu, S.; Chen, J.; Niu, Y.; Zhou, L. A-methylacyl-CoA racemase (AMACR) and prostate-cancer risk: A meta-analysis of 4385 participants. PLOS ONE 2013, 8, e74386.
Johnson, J. R.; Woods-Burnham, L.; Hoker, S. E., Jr.; Batai, K.; Kittles, R. A. Genetic Contributions to Prostate Cancer Disparities in Men of West African Descent. Front. Oncol. 2021, 11, 770500.
Kanwal, R.; Plaga, A. R.; Liu, X.; Shukla, G. C.; Gupta, S. MicroRNAs in prostate cancer: Functional role as biomarkers. Cancer Lett. 2017, 407, 9-20.
Li, W.; Chang, J.; Wang, S.; Liu, X.; Peng, J.; Huang, D.; Sun, M.; Chen, Z.; Zhang, W.; Guo, W.; et al. miRNA-99b-5p suppresses liver metastasis of colorectal cancer by down-regulating mTOR. Oncotarget 2015, 6, 24448-24462.
Liu, J.; Brown, R. E. Morphoproteomics demonstrates activation of mammalian target of rapamycin pathway in papillary thyroid carcinomas with nuclear translocation of MTOR in aggressive histological variants. Mod. Pathol. 2011, 24, 1553-1559.
Ludwig, N.; Werner, T. V.; Backes, C.; Trampert, P.; Gessler, M.; Keller, A.; Lenhof, H. P.; Graf, N.; Meese, E. Combining miRNA and mRNA Expression Profiles in Wilms Tumor Subtypes. Int. J. Mol. Sci. 2016, 17, 475.
Luo, J.; Zha, S.; Gage, W. R.; Dunn, T. A.; Hicks, J. L.; Bennett, C. J.; Ewing, C. M.; Platz, E. A.; Ferdinandusse, S.; Wanders, R. J.; et al. Alpha-methylacyl-CoA racemase: A new molecular marker for prostate cancer. Cancer Res. 2002, 62, 2220-2226.
Mossmann, D.; Park, S.; Hall, M. N. mTOR signalling and cellular metabolism are mutual determinants in cancer. Nat. Rev. Cancer 2018, 18, 744-757.
Murayama, T.; Inokuchi, M.; Takagi, Y.; Yamada, H.; Kojima, K.; Kumagai, J.; Kawano, T.; Suihara, K. Relation between outcomes and localisation of p-mTOR expression in gastric cancer. Br. J. Cancer 2009, 100, 782-788.
Niture, S.; Tricoli, L.; Qi, Q.; Gadi, S.; Hayes, K.; Kumar, D. MicroRNA-99b-5p targets mTOR/AR axis, induces autophagy and inhibits prostate cancer cell proliferation. Tumour Bio. 2022, 44, 107-126.
Pham, V. V.; Zhang, J.; Liu, L.; Truong, B.; Xu, T.; Nguyen, T. T.; Li, J.; Le, T. D. Identifying miRNA-mRNA regulatory relationships in breast cancer with invariant causal prediction. BMC Bioinform. 2019, 20, 143.
Popova, N. V.; Jucker, M. The Role of mTOR Signaling as a Therapeutic Target in Cancer. Int. J. Mol. Sci. 2021, 22, 1743.
Rayford, W.; Beksac, A. T.; Alger, J.; Alshalalfa, M.; Ahmed, M.; Khan, I.; Falagario, U. G.; Liu, Y.; Davicioni, E.; Spratt, D. E.; et al. Comparative analysis of 1152 African-American and European-American men with prostate cancer identifies distinct genomic and immunological differences. Commun. Biol. 2021, 4, 670.
Roudsari, N. M.; Lashgari, N. A.; Momtaz, S.; Abaft, S.; Jamali, F.; Safacipour, P.; Narimisa, K.; Jackson, G.; Bishaycc, A.; Rezaci, N.; et al. Inhibitors of the PI3K/Akt/mTOR Pathway in Prostate Cancer Chemoprevention and Intervention. Pharmaceutics 2021, 13, 1195.
Rubin, M. A.; Zhou, M.; Dhanasekaran, S. M.; Varambally, S.; Barrette, T. R.; Sanda, M. G.; Pienta, K. J.; Ghosh, D.; Chinnaiyan, A. M. alpha-Methylacyl coenzyme A racemase as a tissue biomarker for prostate cancer. JAMA 2002. 287, 1662-1670.
Seo, J.; Jin, D.; Choi, C. H.; Lee, H. Integration of MicroRNA, mRNA, and Protein Expression Data for the Identification of Cancer-Related MicroRNAs. PLOS ONE 2017, 12, e0168412.
Shorning, B. Y.; Dass, M. S.; Smalley, M. J.; Pearson, H. B. The PI3K-AKT-mTOR Pathway and Prostate Cancer: At the Crossroads of AR, MAPK, and WNT Signaling. Int. J. Mol. Sci. 2020, 21, 4507.
Siegel, R. L.; Miller, K. D.; Fuchs, H. E.; Jemal, A. Cancer statistics, 2022. CA Cancer J. Clin. 2022, 72, 7-33.
Sun, D.; Lee, Y. S.; Malhotra, A.; Kim, H. K.; Matecic, M.; Evans, C.; Jensen, R. V.; Moskaluk, C. A.; Dutta, A. miR-99 family of MicroRNAs suppresses the expression of prostate-specific antigen and prostate cancer cell proliferation. Cancer Res. 2011, 71, 1313-1324.
van de Wijngaart, D. J.; Dubbink, H. J.; Molier, M.; de Vos, C.; Trapman, J.; Jenster, G. Functional screening of FxxLF-like peptide motifs identifies SMARCD1/BAF60a as an androgen receptor cofactor that modulates TMPRSS2 expression. Mol. Endocrinol. 2009, 23, 1776-1786.
Wang, B. D.; Ceniccola, K.; Yang, Q.; Andrawis, R.; Patel, V.; Ji, Y.; Rhim, J.; Olender, J.; Popratiloff, A.; Latham, P.; Lai, Y.; Patierno, S. R.; Lee, N. H. Identification and Functional Validation of Reciprocal microRNA-mRNA Pairings in African American Prostate Cancer Disparities. Clin. Cancer Res. 2015, 21, 4970-4984.
Wang, Z.; Zhao, Z.; Yang, Y.; Luo, M.; Zhang, M.; Wang, X.; Liu, L.; Hou, N.; Guo, Q.; Song, T.; et al. MiR-99b-5p and miR-203a-3p Function as Tumor Suppressors by Targeting IGF-1R in Gastric Cancer. Sci. Rep. 2018, 8, 10119.
Yoshida, Y.; Kurokawa, T.; Horiuchi, Y.; Sawamura, Y.; Shinagawa, A.; Kotsuji, F. Localisation of phosphorylated mTOR expression is critical to tumour progression and outcomes in patients with endometrial cancer. Eur. J. Cancer 2010, 46, 3445-3452.
Zhao, Y. J.; Song, X.; Liu, L.; Tang, Y.; Song, X.; Xie, L. Circulating Exosomal miR-150-5p and miR-99b-5p as Diagnostic Biomarkers for Colorectal Cancer. Front. Oncol. 2019, 9, 1129.
Zou, Z.; Tao, T.; Li, H.; Zhu, X. mTOR signaling pathway and mTOR inhibitors in cancer: Progress and challenges. Cell Biosci. 2020, 10, 31.

What is claimed is:

1. A method of treating cancer in a subject in need thereof, the method comprising administering to the subject a therapeutically effective amount of an agonist of miR-34a-5p, an agonist of miR-99b-5p, or an antagonist of miR-96-5p.

2. The method of claim 1, wherein the agonist of miR-34a-5p comprises a miR-34a-5p mimic, wherein the agonist of miR-99b-5p comprises a miR-99b-5p mimic, or wherein the antagonist of miR-96-5p comprises a miR-96-5p antagomir.

3. The method of claim 2, wherein the miR-34a-5p mimic comprises a nucleotide sequence having at least about 80% identity to SEQ ID NO: 1, wherein the miR-99b-5p mimic comprises a nucleotide sequence having at least about 80% identity to SEQ ID NO: 2, or wherein the miR-96-5p antagomir comprises a nucleotide sequence having at least about 80% complementary to SEQ ID NO: 3.

4. The method of claim 1, wherein the administering decreases expression of HIF1A, IGFBP2, and PIK3CB, decreases expression of MTOR, or increases expression of MAPKAPK2 in the subject.

5. The method of claim 1, wherein the subject has prostate cancer, breast cancer, lung cancer, or colon cancer.

6. The method of claim 1, wherein the subject has castration-resistant prostate cancer.

7. The method of claim 1, wherein the subject is a human.

8. The method of claim 1, wherein the subject is of African ancestry.

9. The method of claim 1, further comprising administering to the subject an anti-cancer therapy.

10. The method of claim 9, wherein the anti-cancer therapy comprises chemotherapy, radiotherapy, immunotherapy, surgical resection, or gene therapy.

11. The method of claim 10, wherein the chemotherapy comprises docetaxel.

12. A method of identifying a subject having or at risk of developing cancer, the method comprising assessing the level of miR-34a-5p, miR-99b-5p, or miR-96-5p in a sample from the subject.

13. The method of claim 12, wherein decreased miR-34a-5p, decreased miR-99b-5p, or increased miR-96-5p as compared to a control is indicative that the subject has or is at risk of developing prostate cancer, breast cancer, lung cancer, or colon cancer.

14. The method of claim 12, wherein decreased miR-34a-5p, decreased miR-99b-5p, or increased miR-96-5p as compared to a control is indicative that the subject has or is at risk of developing an aggressive form of cancer.

15. The method of claim 12, wherein each of miR-34a-5p, miR-99b-5p, and miR-96-5p are assessed.

16. The method of claim 12, further comprising assessing the level of HIF1A, IGFBP2, PIK3CB, MTOR, or MAPKAPK2 in the sample.

17. The method of claim 12, wherein the subject is a human.

18. The method of claim 12, wherein the subject is of African ancestry.

19. The method of claim 12, further comprising administering a therapeutically effective amount of an agonist of miR-34a-5p, an agonist of miR-99b-5p, or an antagonist of miR-96-5p to a subject identified as having or at risk of developing cancer.

20. The method of claim 19, wherein the agonist of miR-34a-5p comprises a miR-34a-5p mimic, wherein the agonist of miR-99b-5p comprises a miR-99b-5p mimic, or wherein antagonist of miR-96-5p comprises a miR-96-5p antagomir.

21. The method of claim 12, further comprising administering an anti-cancer therapy to a subject identified as having or at risk of developing cancer.

22. A pharmaceutical composition comprising a miR-34a-5p mimic, a miR-99b-5p mimic, or a miR-96-5p antagomir; and a pharmaceutically acceptable carrier.

23. The pharmaceutical composition of claim 22, wherein the miR-34a-5p mimic comprises a nucleotide sequence having at least about 80% identity to SEQ ID NO: 1, wherein the miR-99b-5p mimic comprises a nucleotide sequence having at least about 80% identity to SEQ ID NO: 2, or wherein the miR-96-5p antagomir comprises a nucleotide sequence having at least about 80% complementary to SEQ ID NO: 3.

24. The pharmaceutical composition of claim 22, comprising the miR-34a-5p mimic, the miR-99b-5p mimic, and the miR-96-5p antagomir.


SEQUENCE LISTING (19 sequences)

SEQ ID NO: 1 - RNA, 22 nt, Homo sapiens
  source 1..22: mol_type = other RNA
tggcagtgtcttagctggttgt

SEQ ID NO: 2 - RNA, 22 nt, Homo sapiens
  source 1..22: mol_type = other RNA
cacccgtagaaccgaccttgcg

SEQ ID NO: 3 - RNA, 23 nt, Homo sapiens
  source 1..23: mol_type = other RNA
tttggcactagcacatttttgct

SEQ ID NO: 4 - DNA, 22 bp, synthetic construct
  source 1..22: mol_type = other DNA
tggcagtgtcttagctggttgt

SEQ ID NO: 5 - DNA, 23 bp, synthetic construct
  source 1..23: mol_type = other DNA
tttggcactagcacatttttgct

SEQ ID NO: 6 - DNA, 22 bp, synthetic construct
  source 1..22: mol_type = other DNA
cacccgtagaaccgaccttgcg

SEQ ID NO: 7 - DNA, 23 bp, synthetic construct
  source 1..23: mol_type = other DNA
agcagcattgtacagggctatga

SEQ ID NO: 8 - DNA, 20 bp, synthetic construct
  source 1..20: mol_type = other DNA
catgtcagggctggtctttt

SEQ ID NO: 9 - DNA, 20 bp, synthetic construct
  source 1..20: mol_type = other DNA
gcacttttccagctttcctg

SEQ ID NO: 10 - DNA, 20 bp, synthetic construct
  source 1..20: mol_type = other DNA
cccaatggatgatgacttcc

SEQ ID NO: 11 - DNA, 20 bp, synthetic construct
  source 1..20: mol_type = other DNA
tgggtaggagatggagatgc

SEQ ID NO: 12 - DNA, 20 bp, synthetic construct
  source 1..20: mol_type = other DNA
ccctcaagtcgggtatgaag

SEQ ID NO: 13 - DNA, 20 bp, synthetic construct
  source 1..20: mol_type = other DNA
acctggtccagttcctgttg

SEQ ID NO: 14 - DNA, 20 bp, synthetic construct
  source 1..20: mol_type = other DNA
cctcacaagacatcgctgaa

SEQ ID NO: 15 - DNA, 20 bp, synthetic construct
  source 1..20: mol_type = other DNA
tccggctgctgtagcttatt

SEQ ID NO: 16 - DNA, 20 bp, synthetic construct
  source 1..20: mol_type = other DNA
agaagtgctgggtccagaga

SEQ ID NO: 17 - DNA, 20 bp, synthetic construct
  source 1..20: mol_type = other DNA
aattcatactggcccattcg

SEQ ID NO: 18 - DNA, 20 bp, synthetic construct
  source 1..20: mol_type = other DNA
gtactggagaggggagagca

SEQ ID NO: 19 - DNA, 20 bp, synthetic construct
  source 1..20: mol_type = other DNA
tgaagctgagacaagcagga